(19) United States
(12) Patent Application Publication
(10) Pub. No.: US 2019/0167723 A1
(43) Pub. Date: Jun. 6, 2019

(54) SERUM FRACTION OF PLATELET-RICH FIBRIN AS A CELL CULTURE ADDITIVE

(71) Applicant: Lacerta Technologies Inc., Raleigh, NC (US)

(72) Inventors: Zsombor LACZA, Csopak (HU); Gabriella VÁCZ, Paks (HU); István HORNYÁK, Krems an der Donau (AT); Melinda SIMON, Budapest (HU); Olga KUTÉN, Krems an der Donau (AT); Vivek JEYAKUMAR, Krems an der Donau (AT)

(21) Appl. No.: 16/173,430

(22) Filed: Oct. 29, 2018

Related U.S. Application Data
(63) Continuation-in-part of application No. 15/283,576, filed on Oct. 3, 2016, now Pat. No. 10,111,906.
Application No. 15/283,576 is a division of application No. 14/178,573, filed on Feb. 12, 2014, now Pat. No. 9,480,716.
Application No. 14/178,573 is a continuation-in-part of application No. PCT/US2017/020926, filed on Mar. 6, 2017.
Application No. PCT/US2017/020926 is a continuation-in-part of application No. PCT/US2017/031585, filed on May 8, 2017.
(60) Provisional application No. 61/763,504, filed on Feb. 12, 2013.

(30) Foreign Application Priority Data
Mar. 4, 2016 (HU) P1600180
May 6, 2016 (HU) P1600306

(51) Int. Cl. A61K 35/19; A61L 27/52; A61L 27/36; A61K 35/16; A61L 27/38

(52) U.S. Cl. CPC A61K 35/19; A61L 27/52; A61L 27/3616; A61L 2400/06; A61L 27/38; A61L 2430/02; A61K 35/16

(57) ABSTRACT

A method of preparing an isolated serum fraction of platelet rich fibrin, cell cultures comprising said serum fraction and its use as a cell culture additive. The invention also relates to increasing proliferation rate of chondrocytes, to the treatment of articular or joint diseases and to increasing the proliferation rate of mesenchymal stem cells. The isolated serum fraction of platelet rich fibrin (PRF), is prepared by providing platelet rich plasma (PRP) without the addition of an anticoagulant; clotting the PRP to obtain a coagel of PRF; and separating the coagel to isolate the serum fraction which comprises an activated platelet releasate; and further provides for the isolated serum fraction obtained by such method, and its medical use.

### PRIOR APPLICATIONS

The present application is a continuation-in-part (CIP) of application Ser. No. 15/283,576 (published as US 2017/0035809 A1), filed Oct. 3, 2016 (now U.S. Pat. No. 10,111,906), which is in turn a division of application Ser. No. 14/178,573, filed on Feb. 12, 2014 (now U.S. Pat. No. 9,480,716), claiming priority from Provisional application No. 61/763,504, filed on Feb. 12, 2013; and is also a CIP of PCT/US2017/020926 filed on Mar. 6, 2017 (published as WO 2017/152172) and claiming priority from HU P1600180 filed on Mar. 4, 2016 as well as a CIP of PCT/US2017/031585 filed on May 8, 2017 (published as WO 2017/193134) and claiming priority from HU P1600306 filed on May 6, 2016; wherein the content of each of said patent applications is incorporated herein by reference.

### FIELD OF THE INVENTION

The invention refers to a method of preparing an isolated serum fraction of platelet rich fibrin, cell cultures comprising said serum fraction and its use as a cell culture additive. The invention also relates to a novel method for increasing proliferation rate of chondrocytes and for the treatment of articular or joint diseases. The invention relates to a new method for increasing proliferation rate of mesenchymal stem cells.

### BACKGROUND

In vitro cell culture or maintenance of eukaryotic cells still requires utmost care and a careful selection of medium. Fetal bovine serum (FBS) or fetal calf serum (FCS) is still possibly the most widely used serum-supplement for such purpose, due to a high level of growth factors and a low level of antibodies. The supplementation of cell culture media with animal serum product is known to be essential for cell growth and for proliferation.

FBS is, however, to a certain extent indefinite; its composition varies depending on source, and comprises a large number of undefined proteins that can lead to unwanted stimulation of cells. In human-related use further concerns arise from the fact that FBS is of animal origin. Besides the need for efforts towards a standardization of cell culture protocols, considerable ethical concerns were raised about collection of FBS for example because a very large number of bovine fetuses have to be harvested in order to meet market need [Gstraunthaler, Gerhard Alternatives to the Use of Fetal Bovine Serum: Serum-free Cell Culture. ALTEX 2003 20, 4/03 275-281]. In spite of its disadvantages FBS is still used in therapeutic applications and in clinical trials, though its use is highly regulated in particular in regenerative medicine [van der Valk, J. et al. The humane collection of fetal bovine serum and possibilities for serum-free cell and tissue culture. Toxicol In Vitro. 2004. 18(1):1-12].

Other known blood separation products can be evaluated for their potential role as cell media supplement. Plasma is the anticoagulated, centrifuged whole blood supernatant, which has the disadvantage of containing anticoagulant (e.g. EDTA, heparin or citrate derivatives), which affects enzymatic balance and interfere with systemic blood coagulation as well and plasma also contains fibrinogen, which is converted to fibrin just like in PRP and causes limited protein transport. Another possible candidate is serum, which is the supernatant of the coagulated whole blood. This has the same disadvantage as ACS (autologous conditioned serum) namely that during the clotting of whole blood inflammatory markers are populated in a similar manner as in a systemic inflammatory. Usually the longer the blood is coagulating, the more inflammatory cytokines are produced, which unfortunately can lead to a positive feedback loop so ultimately this can generate inflammation if injected back to the patient. In summary, as a supplementing material the main criteria are to have a somewhat standardized, fibrin and/or fibrinogen and anticoagulant free autologous blood separation product, which does not induce inflammation.

Gstraunthaler, Gerhard [Gstraunthaler, Gerhard, 2003, infra] discusses alternatives to the use of FBS and focuses on serum-free (SF) cell cultures. The author reviews the art and concludes that growing cells in various serum free media often has the advantage of high specificity and thus serum-free media allows the selection of certain cell types and their specific stimulation and differentiation. However, he is rather gloomy about a general-purpose serum-free medium which “has not yet been developed and is almost certainly an unattainable goal”.

Moreover, development of SF media strongly depends on the cell type and the culture system, and is often a difficult task despite some existing methods [van der Valk, J. et al. Optimization of chemically defined cell culture media. Toxicol In Vitro. 2010. 24(4):1053-63].

In case of a variant of serum free media individual growth factors are added to replace growth factors present in FBS.

Platelets or thrombocytes in mammals are small, irregularly shaped cell-like compartments in blood without a nucleus, which are derived from precursor megakaryocytes. Platelets play a fundamental role in hemostasis. Platelets isolated from peripheral blood are an autologous source of growth factors. Platelet concentrates are blood-derived products traditionally used for example to treat consequences of thrombopenia. It has long been recognized that several components in blood are a part of the natural healing process and can accelerate healing when added to surgical sites. In the art various platelet concentrates have been used to accelerate soft-tissue and hard-tissue healing.

Fibrin glue is formed by polymerizing fibrinogen with thrombin and calcium. It was originally prepared using donor plasma; however, because of the low concentration of fibrinogen in plasma, the stability and quality of fibrin glue were low.

Platelet rich plasma (PRP) in a sense is an autologous modification of fibrin glue, which has been described and used in various applications with apparent clinical success. PRP obtained from autologous blood is used to deliver growth factors in high concentrations to the site of bone defect or a region requiring augmentation. Platelet-rich plasma (PRP) is an easily accessible source of growth factors to support bone- and soft-tissue healing. It is typically derived from anticoagulated blood by methods that concentrate autologous platelets and is added to surgical wounds or grafts and to other injuries in need of supported or accelerated healing. A blood clot is in the focus of initiating any soft-tissue healing and bone regeneration. In all natural wounds, a blood clot forms and starts the healing process. PRP is a simple strategy to concentrate platelets or enrich natural blood clot, which forms in normal surgical wounds, to initiate a more rapid and complete healing process. A natural blood clot contains 95% red blood cells, 5% platelets, less than 1% white blood cells, and numerous amounts of fibrin strands. A PRP blood clot, which also may be called as a type of platelet-rich fibrin (PRF) contains typically 4% red blood cells, 95% platelets, and 1% white blood cells.

Positive effects of PRP in dental tissue repair and in other maxillofacial cases are widely exploited. PRP is also applied for the treatment of other pathologies such as osteoarthritis, tendinitis and nerve injury and is gaining traction as a ‘cure-all’ for many musculoskeletal diseases. However, the exact mode of action is unknown and the general perception of PRP is that both the protocols and the results are highly variable.

Studies into clinical efficiency of PRP are not conclusive though and one of the main reasons for this is that different PRP preparations are used, eliciting different responses that cannot be compared. Amable, P. R. et al. [Amable, P. R. et al. Platelet-rich plasma preparation for regenerative medicine: optimization and quantification of cytokines and growth factors. Stem Cell Research & Therapy, 2013, 4:67] suggested a standardized PRP and the use of PRP in therapies aiming for tissue regeneration, and its content characterization will allow us to understand and improve the clinical outcomes.

While the use of PRP in bone healing does have a sound scientific basis, its application appears only beneficial when used in combination with osteoconductive scaffolds. Aggressive processing techniques and very high concentrations of PRP may not improve healing outcomes.

Moreover, many other variables exist in PRP preparation and use that influence its efficacy; the effect of these variables should be understood when considering PRP as a therapeutic measure.

Anitua et al. [Anitua, E. et al. New insights into and novel applications for platelet-rich fibrin therapies. TRENDS in Biotechnology, 2006, 24(5): 227-234.] also summarize knowledge about PRP and teach that platelet rich plasma, i.e. PRP itself has been described to enable MSC proliferation without deforming cell structure. However, Anitua does not teach the use of any serum fraction of platelet rich fibrin in an in vitro culture.

Several techniques for platelet concentrates are available and their application may be confusing because each method leads to a different product with different biology and potential uses.

WO2010/089379A1 describes the combination of anticoagulated (soluble) platelet rich plasma (PRP) with a coagulation factor to activate PRP when administering the combination to a patient.

US2009/0047242A1 describes a conditioned blood composition which is prepared by incubating blood in a vessel that has a specific surface area to induce factors and cytokines, such as interleukin-6.

WO2010/02047A1 describes a blood product comprising fibrin, thrombocytes and leukocytes, which is obtained by surface activation of blood coagulation.

WO2007/127834A2 discloses a thrombin composition obtained by contacting whole blood, a component thereof or fraction thereof with a contact activation agent, such thrombin composition containing a stabilizing agent, such as ethanol.

Human platelet lysate has been suggested as a serum substitute for fetal bovine serum in cell culture media [Rauch, C. et al. Alternatives to the use of fetal bovine serum: human platelet lysates as a serum substitute in cell culture media. ALTEX. 2011; 28:305-16]. Platelet lysates are prepared from platelet concentrates (or platelet rich plasma) by isolating platelets and lysing the platelets preferably by multiple freeze/thawing cycles and centrifugation. As platelet lysates (PL) comprise plasma with fibrinogen and all other clotting factors, addition of anticoagulants is unavoidable to prevent gelatinization of hPL medium; moreover, the preparation of PL needs standardization [Hemeda, H. et al. Evaluation of human platelet lysate versus fetal bovine serum for culture of mesenchymal stromal cells. Cytotherapy 2014; 16: 170-180].

Platelet-rich fibrin (PRF) belongs to a new generation of platelet concentrates allowing a simplified processing and handling. The slow polymerizing PRF membrane is particularly favorable to support the healing process, however, the biology behind the effect of PRF is still largely unknown and it is only suggested that the effect is due to certain soluble molecules that are most likely trapped in fibrin meshes of PRF. PRF is also used in combination with freeze-dried bone allograft to enhance bone regeneration in sinus floor elevation.

As serum free media necessitated the addition of individual growth factors, an idea that certain growth factors obtained from a platelet-rich fibrin scaffold can be used as a substitute for FCS has also been raised [Anitua, E. et al. New insights into and novel applications for platelet-rich fibrin therapies. TRENDS in Biotechnology, 2006, 24(5): 227-234].

Burnouf, T. et al. [Burnouf, Thierry et al. Human blood-derived fibrin releasates: Composition and use for the culture of cell lines and human primary cells. Biologicals (2012), 40: 21-30] have prepared blood derived preparations from volunteer donors, wherein non-anticoagulated blood was centrifuged at 700 g to isolate a supernatant serum (SS) and a platelet-rich fibrin (PRF) clot which was squeezed to extract the releasate (PRFR). Cell growth promoting activity of pooled SS and PRFR at 1, 5, and 10% in growth medium was evaluated over 7 days using human (HEK293, MG-63) and animal (SIRC, 3T3) cell lines and two human primary cultures (gingival fibroblasts and periodontal ligaments). Viable cell count was compared to that in cultures in FBS free-medium and 10% FBS supplement. SS and PRFR at 1-10% stimulated cell growth significantly more than FBS-free medium and in several cases similarly to 10% FBS.

Mesenchymal stem cells are increasingly important type of cells in proposed medical applications, in particular in regenerative medicine.

Mesenchymal stem cells (MSCs) are defined as multipotent, self-renewing, non-hematopoietic cells, which originate from the mesoderm and characterized by typical surface markers, for example ALCAM (activated leukocyte cell adhesion molecule, CD166) and STRO-1. Their multipotency permits the differentiation to bone, cartilage, reticular tissues and fat. Due to their advantageous properties MSCs have been proved to be effective as autologous cell transplantation in clinical trials in case of regeneration of periodontal tissue defects, diabetic critical limb ischemia, bone damage caused by osteonecrosis and burn-induced skin defects. However, MSCs multi-lineage potential can be lost easily, when MSCs are grown in vitro on standard tissue culture plastics. Their proliferation and multilineage differentiation potential also decreases with aging or increased time in in vitro culture.

This phenomenon is the major obstacle to the clinical application of MSCs, because the patient's own stem cells cannot be harvested and expanded without phenotypical change. MSCs cultured and propagated ex vivo lose their regenerative capacity,—in case of bone—their ability to augment and promote bone formation. In addition, MSCs are usually cultured in FBS-supplemented medium, which provides a xenogeneic additive to the reintroduced cell population [Sotiropoulou, P. A. et al. Characterization of the Optimal Culture Conditions for Clinical Scale Production of Human Mesenchymal Stem Cells. Stem Cells, February 2006, Volume 24, Issue 2, Pages 462-471]. This is often the case even when used for human stem cell therapy wherein FBS as an animal derived product is otherwise not advantageous.

FBS can trigger immune reactions, and due to its unpredictable lot-to-lot variability these effects are totally incidental. Therefore, for the translation of stem cells to clinical uses, it would be ideal to evolve xeno-free culture conditions. Among the nowadays applied human blood separation products; platelet-rich plasma (PRP) has already been proven in different clinical scenarios, such as orthopedics, ophthalmology and healing therapies, as a growth factor pool for improving tissue regeneration. Studies into its clinical efficiency are not conclusive and one of the main reasons for this is that different PRP preparations are used, eliciting different responses that cannot be compared. Amable P. R. et al. (Amable, P. R. et al. Platelet-rich plasma preparation for regenerative medicine: optimization and quantification of cytokines and growth factors. Stem Cell Research & Therapy 2013, 4:67; Rigotti, G. et al. Expanded Stem Cells, Stromal-Vascular Fraction, and Platelet-Rich Plasma Enriched Fat: Comparing Results of Different Facial Rejuvenation Approaches in a Clinical Trial. Aesthet Surg J. 2016; 36(3):261-70) suggested a standardized PRP and the use of PRP in therapies aiming for tissue regeneration, and its content characterization will allow us to understand and improve the clinical outcomes. Uncertainties of the process are involved.

Chondrocytes are a type of cells which are particularly difficult to be propagated in vitro, in particular in a form which renders them suitable for regenerative use for example in osteoarthritis. Osteoarthritis is a degenerative joint disease and the repair of osteochondral defects yet remains a challenge due to its poor regeneration capacity. Fortier, L. A. et al. [Fortier, L. A. et al. The Effects of Platelet-Rich Plasma on Cartilage: Basic Science and Clinical Application. Operative Techniques in Sports Medicine, 2011, 19(3): 154-159] teach that in preclinical animal model studies, PRP slows the progression of osteoarthritis, but there are mixed results after the use of PRP to facilitate the repair of chondral or osteochondral defects. PRP-bone marrow-derived stromal cell constructs aided in the repair of chondral defects and positive results were also shown 1 year after intra-articular injection in patients suffering from knee pain. Although most studies support the clinical use of PRP for the treatment of cartilage injury and joint pain the authors suggest that more extensive testing and reporting seems to be necessary.

The human body's own cartilage is still the best material for lining knee joints. This drives efforts to develop ways of using cells of the same species or the subject's own cells to grow, or re-grow cartilage tissue to replace missing or damaged cartilage. One cell-based replacement technique is called autologous chondrocyte implantation (ACI) or autologous chondrocyte transplantation (ACT). A review evaluating autologous chondrocyte implantation (ACI) was published in 2010. The conclusions are that it is an effective treatment for full thickness chondral defects. The evidence does not suggest ACI is superior to other treatments [Vasiliadis, H. S. et al. (2010). Autologous chondrocyte implantation for the treatment of cartilage lesions of the knee: a systematic review of randomized studies. Knee Surgery, Sports Traumatology, Arthroscopy, 2010, 18(12): 1645-1655].

These autologous cells may be expanded in vitro before implantation in cell culture medium that contains serum for supporting the proliferation of the cells. The process required increasing the rate of proliferation.

It has been suggested already in 2006 that PRP isolated from autologous blood may be useful as a source of anabolic growth factors for stimulating chondrocytes to engineer cartilage tissue [Akeda, K. et al. Platelet-rich plasma stimulates porcine articular chondrocyte proliferation and matrix biosynthesis. OsteoArthritis and Cartilage, 2006, 14(12): 1272-1280]. The authors have observed that treatment with PRP growth factors did not markedly affect the types of proteoglycans and collagens produced by porcine chondrocytes, suggesting that the cells remained phenotypically stable in the presence of PRP.

Further uncertainties of the process are involved. Proliferation in vitro often includes redifferentiation. It would be desirable to have a method, in order to have better regulatability of the process, which promotes proliferation of chondrocytes without promoting differentiation.

Mobasheri, A. et al. [Mobasheri, A. et al. Chondrocyte and mesenchymal stem cell-based therapies for cartilage repair in osteoarthritis and related orthopaedic conditions. Maturitas, 2014, 78: 188-198] review the challenges associated with cartilage repair and regeneration using cell-based therapies that use chondrocytes and mesenchymal stem cells (MSCs) and explore common misconceptions associated with cell-based therapy and highlight a few areas for future investigation. The authors, somewhat gloomily conclude that cell-based therapies may be unrealistic options of the osteoarthritic lesions due to their complex geometry, in contrast to the more local focal defects that may be seen in younger (i.e. athletic patients) and suggest that further basic research is needed. Clearly, in many cases cell-based therapies may not be suitable or effective for end-stage OA.

Peterson, L. et al. [Peterson, L. et al. Autologous Chondrocyte Implantation: A Long-term Follow-up. Am J Sports Med, 2010, 38: 1117-24] report a study which suggests that the clinical and functional outcomes remain high even 10 to 20 years after the implantation.

The present inventors have found that problems raised in the art may be solved by a newly developed serum fraction of platelet rich fibrin (called herein SPRF) having a surprisingly high regenerative potential in the culturing of cells.

SPRF does not contain fibrinogen, anticoagulants and the inflammation markers are low.

SPRF significantly improved the proliferation capacity of osteoblast cells damaged by ischemia.

After testing it as a stem cell medium supplement, the inventors have surprisingly found that use of a serum from platelet rich fibrin (SPRF) instead of PRP and FBS enhanced the proliferation rate of human mesenchymal stem cells in vitro while phenotypical changes were not observed and differentiation potential of proliferated MSCs was maintained. Moreover, culturing human subchondral bone pieces in SPRF supplemented medium cell viability was not only retained, but also significantly increased in 7-days culture without any measurable cell differentiation. The inventors revealed that predominantly mesenchymal stem cells were multiplicated in the course of the incubation time.

Fibrin releasates apparently have not been used in the art for the culturing including maintenance of MSCs.

SPRF also increased the proliferation rate of chondrocytes, preferably chondrocytes in vitro, in particular dedifferentiated chondrocytes.

The inventors have surprisingly found that SPRF increased the proliferation rate of dedifferentiated, preferably osteoarthritic chondrocytes to a larger extent than PRP, and also to a larger extent than FCS.

### Abbreviations

ACS (autologous conditioned serum)

ACI (autologous chondrocyte implantation)

AD-MSCc (adipose derived mesenchymal stem cells)

BM-MSCs (bone marrow derived mesenchymal stem cells)

DMEM (Dulbecco's modified Eagle's medium)ECM (extracellular matrix)

FBS (fetal bovine serum)

FCS (fetal calf serum)

FGF (fibroblast growth factor)

hMSCs (human mesenchymal stem cells)

hSBPs (human subchondral bone pieces)

MSCs (mesenchymal stem cells)

OA (osteoarthritis)

PPP (platelet poor plasma)

PRF (platelet rich fibrin)

PRP (platelet rich plasma)

SPRF (serum fraction of platelet rich fibrin)

### BRIEF SUMMARY OF THE INVENTION

It is an object of the invention to provide for an improved blood preparation, particularly as an additive or supplement to cell culture media. The cell cultures supplemented by the improved blood preparation may have several uses, among others in medicine.

The object is solved by the subject of the inventions disclosed herein.

In an aspect, the serum fraction of the invention is provided for in vitro use as a cell culture additive.

According to the invention the preparation can be prepared by a method of preparing an isolated serum fraction of platelet rich fibrin (PRF), hereinafter also referred to as SPRF (serum of PRF), comprising the steps of

a. providing platelet rich plasma (PRP) without the addition of an anticoagulant;

b. clotting the PRP to obtain a coagel of PRF; and

c. separating the coagel to isolate the serum fraction which comprises an activated platelet releasate.

In a preferred embodiment, method steps a. and b. may be carried out in a single step procedure, e.g. wherein PRP is produced from a blood sample by fractionation during which the coagel is formed, e.g. by active activation of coagulation or self-activation of coagulation. The serum fraction can be separated by pressing, squeezing, filtering and/or centrifuging the coagel to isolate the serum fraction containing the fluid fraction of PRF.

More preferably, method steps a., b. and c. may be carried out in a single step procedure, e.g. in a closed system.

In an embodiment the invention relates to a cell culture comprising
mammalian cells,
a cell culture medium,
a serum fraction containing the fluid fraction of platelet-rich fibrin, i.e. serum fraction of PRF (SPRF),

wherein
said SPRF being obtained by a method comprising the steps of
a. separating and removing the red blood cell fraction from a venous blood sample without the addition of an anticoagulant to provide a plasma, /OR
to provide a platelet rich plasma without the addition of an anticoagulant;
b. clotting said plasma to obtain a coagel of PRF spontaneously by centrifugation carried out at 1000 to 5000 g and a supernatant, wherein in said method the centrifugation is carried out for 2 to 20 minutes;
c. pressing or squeezing the coagel to obtain fluid fraction from the coagel, thereby obtaining said SPRF;

wherein said SPRF is added to the medium,

said SPRF comprising a platelet releasate from activated platelets and

said SPRF comprising a reduced content of red blood cells, platelets or fibrinogen as compared to whole blood or a reduced content of fibrin as compared to said plasma, and

wherein said SPRF is capable of inducing cell proliferation or restoring cell proliferation capacities.

In particular, the method of preparing the isolated serum fraction of platelet rich fibrin (SPRF), comprises the steps of

a. providing platelet rich plasma without the addition of an anticoagulant;

b. clotting the plasma to obtain a coagel of PRF spontaneously by centrifugation carried out at 1000 to 5000 g;

c. pressing or squeezing the coagel to separate the serum fraction which comprises an activated platelet releasate from the coagel, thereby obtaining an isolated serum fraction containing the fluid fraction of PRF.

In an embodiment the “serum fraction of platelet rich fibrin”, hereinafter also referred to as SPRF (serum of platelet rich fibrin) is a serum fraction as defined or described in U.S. application Ser. No. 14/178,573, filed on Feb. 12, 2014 (now U.S. Pat. No. 9,480,716) which is incorporated herein by reference. Preferably SPRF, as used herein is isolated from whole blood obtained from donors by centrifugation to obtain a fibrin clot and wherein the fibrin clot (platelet rich fibrin) is pressed or squeezed to obtain the SPRF as an exudate or releasate of the fibrin clot. Preferably, said centrifugation to obtain the fibrin clot is carried out at 1000 to 4000 g, preferably 1000 to 3000 g or 1500 to 2500 g or 1000 to 2500 g or more preferably 1500 to 2000 g for 2 to 20 minutes or 3 to 15 minutes or 5 to 15 minutes or 3 to 12 minutes or e.g. 5 or 10 minutes, within 4, 3, 2 or preferably within 2 or 1.5 or within 1 minute(s) from blood collection, and SPRF can be collected and stored e.g. frozen.

Specifically, the coagel is separated by pressing, squeezing, filtering and/or centrifuging the coagel to isolate the serum fraction containing the fluid fraction of platelet rich fibrin.

Preferably, SPRF is obtained by
providing whole blood obtained from the one or more donor subject(s),
centrifuging whole blood at 1000 to 4000 g (or in the ranges as disclosed herein) for 2 to 20 minutes (or in a range as disclosed herein) within 4 minutes from blood collection, thereby providing a fibrin clot (platelet rich fibrin),
pressing or squeezing the fibrin clot to obtain the SPRF as an exudate or releasate thereof.

The meaning of the measure “g” is gravitational acceleration, i.e. the acceleration of an object caused by the force of gravitation on Earth. At different points on Earth, objects fall with an acceleration between 9.764 m/s² and 9.834 m/s² depending on altitude and latitude, with a conventional standard value of exactly 9.80665 m/s² (approximately 32.174 ft/s²), which can be approximated under the present conditions with 10 m/s².

Preferably, pressing or squeezing the coagel to separate the serum fraction is carried out within 2 hours or within 1 hour, or preferably within 30 minutes or 20 minutes, more preferably within 15 minutes or within 10 minutes or within 5 minutes from the end of the centrifuging step.

Preferably the pressing or squeezing is carried out by a device which comprises plunger releasably connected to a piston within a tube. In one embodiment, the device is a syringe having a plunger releasably connected to a piston within a tube. In one embodiment, the plunger and the piston have inter-engaging screw threads. In one embodiment, there is a gap between said connector parts. In one embodiment, the connector first part comprises a plate with a surface facing into the syringe volume. In one embodiment, the plate is dish-shaped with a generally concave surface facing into the syringe volume.

In an embodiment, said plate has an aperture aligned with a passageway in the container second part. In one embodiment, a gap is maintained by spacers of one part engaging in apertures of the other part. In one embodiment, the connector first and second parts are releasably engaged.

In an embodiment, the connector has a Luer fastener suitable for engaging a cannula or the container. In one embodiment, the container has a piston fitted to move in the second part chamber in a direction away from the syringe under applied forces during centrifuging.

In an embodiment, blood is handled by a method of handing blood using said device comprising a syringe for taking a blood sample, and a container with a chamber releasably connected to the syringe, and a fluid passageway between the syringe volume and the chamber, a connector interfacing between the syringe and the container, said connector having said fluid passageway, and said connector comprises a first part forming a base for the syringe, and a second part arranged to connect to the container during centrifuging and to a cannula after removal of the container, the method comprising the steps of:

taking a blood sample in the syringe when the syringe is disconnected from the container,

fastening the container to the syringe,

centrifuging the assembly of the syringe and the container and allowing the coagel (clot) to be formed,

removing the container from the syringe, leaving a first blood fraction within the syringe and a

second blood fraction within the container, and optionally,

squeezing or pressing the coagel by moving the plunger in the piston.

Preferably the device has a design as described in WO 2017/093838.

In one embodiment, the method comprises the further step of releasing the serum fraction from the syringe to a patient. In this embodiment, the centrifuging step results in a clot forming on an inner surface of the device or the clot is formed on a surface around said fluid passageway and by pressing the clot (coagel) the serum fraction is obtained.

According to a specific aspect, the blood sample is collected in a clot device such as a clot tube or clot syringe, optionally wherein the PRP is prepared and clotted to obtain the coagel, e.g. a clot activating tube or syringe, which is typically equipped with appropriate coagulation initiators or accelerators, herein referred to as “coagulation activators”. For example, typical clot tubes may provide a negatively charged contact surface, such as glass, which would accelerate spontaneous clotting of the PRP during separation of the red blood cell fraction. The device may not only be used for collecting the blood, but also for preparing the PRP, e.g. by centrifugation of the blood sample in one or more consecutive steps.

The serum fraction of the invention, hereinafter also referred to as SPRF (serum of PRF), may include the supernatant of the coagel and/or the fluid fraction obtained from the coagel, e.g. SPRF which essentially consists of the fluid fraction. For example, such serum fraction essentially consisting of the PRF fluid fraction is prepared upon fractionating the PRF to isolate the fluid fraction from PRF, e.g. by separating the solid coagel mainly consisting of fibrin gel and platelets. Specifically, upon clotting the PRP and formation of the PRF, the acellular or clear supernatant from the PRF may be isolated, or may be removed before fractionating the PRF to isolate the PRF fluid fraction. Such PRF fluid fraction turned out to contain the highest concentration of activated platelet releasate and growth factors contained therein.

Specifically, the PRP may be obtained from a blood sample from a single donor or from multiple donors and mixed together to obtain a single blood sample. According to a specific aspect, the PRP is obtained from venous blood collected from a single donor.

The blood sample can further be obtained from the same individual who will receive the serum fraction. Thus, the blood and serum fraction can be autologous to the recipient. In a preferred embodiment, autologous venous blood or PRP may be used.

The blood sample can also be obtained from a non-autologous individual or donor or multiple donors. Moreover, the blood sample can be obtained from a heterologous individual or donor or multiple donors. Thus, the blood sample can be obtained from one or more individuals. The serum fraction of the invention which is heterologous may be treated to inactivate or remove possibly present blood borne viruses by conventional virus inactivation or depletion methods, including treatment with solvent and detergent, low pH and/or nanofiltration. Alternatively and/or additionally, the virus safety may be ensured by selecting suitable donors which have been determined not to be infected with blood borne pathogens.

Typically, the volume of such blood sample is ranging between 1 ml to 100 ml, preferably between 10 ml to 40 ml more preferably between 15 ml to 35 ml.

The device may also be suitable for separating specific blood products besides the physico-chemical separation by e.g. filtration or allocating specific blood separation fractions to another chamber or barrel of the device. Optionally, the PRP is prepared and clotted in or by means of such device to obtain the coagel.

For example, a serum fraction of the invention, e.g. an autologous preparation, may be freshly prepared employing bedside methods to collect blood from individual patients, followed by concentration and activation of platelets through coagulation activation.

According to a specific example, the clotting or coagulation activation, herein also called “activation of PRP” is effected by an incubation step during which the coagel is formed, e.g. where the PRP is allowed to stand at room temperature up to 37° C., preferably about 18-25° C., for about 1-8 h, preferably for about 2-6 h.

According to a specific aspect, the serum fraction comprises a platelet releasate from activated platelets.

According to a specific aspect, the platelet releasate is enriched in platelet factors released from the activated platelets, as compared to PRP. Among the platelet factors there is a series of growth factors, cytokines, interleukins, chemokines and angiogenesis or growth factor related proteins.

Preferably, the serum fraction contains specific factors, and is typically characterized by a specific profile of such factors, among them growth factors and cytokines. For example, the serum fraction contains at least one of angiogenesis or growth factor proteins selected from the group consisting of Activin-A, ADAMTS-1, Angiogenin, CXCL16, DPPIV, Endoglin, Endostatin/Collagen XVII, FGF-4, GM-CSF, HB-EGF, HGF, IGFBP-1, IGFBP-2, IGFBP-3, IL-1 β, IL-8, LAP (TσPβ-1), Leptin, MCP-1, MMP-8, MMP-9, NRGI-βI, Pentraxin-3, PD-ECGF, PDGF-AB/PDGF-BB, PIGF, Prolactin, TIMP-4, Thrombospondin-1, uPA, e.g. at a similar (such as 10%, 15%, or less than 20% difference) or different level (such as a change of at least 20%) compared to PRP or whole blood, e.g. measured by a proteome profiler array, ELISA or similar assays. In a preferred embodiment, at least 2 of these factors are present, or at least 3, 4, 5, 6, 7, 8, 9, or 10, or even more, at least 15, 20, 25 or up to all of these factors are present.

In a preferred embodiment, one or more of these factors are enriched in the serum fraction of the invention. The enrichment of the specific factors is typically determined, if the concentration is increased by at least 20%, 30%, 40% or 50%, or even at least 100%, or at least 2-fold, at least 3-fold, or at least 4-fold increased, as compared to PRP or whole blood. In a preferred embodiment, the serum fraction is enriched in at least one of angiogenesis or growth factor related proteins selected from the group consisting of Platelet factor 4, Serpin El, or TIMP-1, as compared to PRP or whole blood, e.g. measured by a proteome profiler array, ELISA or similar assays. In a preferred embodiment, the serum fraction is enriched in one, two, or all three of these selected factors.

According to a further specific embodiment, the serum fraction is characterized by an increased Platelet factor 4 concentration as compared to PRP or whole blood, e.g. at least 2-fold, preferably at least 3-fold, at least 4-fold or 5-fold enriched measured by a proteome profiler, ELISA or similar assays.

In a preferred embodiment, one or more of these factors are depleted in the serum fraction of the invention, i.e. the content or concentration is reduced. Preferably, the serum fraction is depleted in at least one of angiogenesis or growth factor related proteins selected from the group consisting of SDF-1, Angiopoietin-1, EGF, PDGF, VEGF, as compared to PRP or whole blood, e.g. measured by a proteome profiler array, ELISA or similar assays.

According to a specific embodiment, the serum fraction is characterized by a decreased concentration of stromal cell-derived factor 1 (SDF-1, CXCL12) as compared to PRP or whole blood. Preferably, the SDF-1 concentration measured is less than 350 pg/ml, preferably less than 275 pg/ml measured by ELISA.

According to a further specific embodiment, the serum fraction is characterized by a decreased Angiopoietin-1 concentration as compared to PRP or whole blood.

According to a further specific embodiment, the serum fraction is characterized by a decreased Epidermal Growth Factor (EGF) concentration as compared to PRP or whole blood.

According to a further specific embodiment, the serum fraction is characterized by a decreased Platelet derived growth factor (PDGF) concentration as compared to PRP or whole blood.

According to a further specific embodiment, the serum fraction is characterized by a decreased Vascular Endothelial Growth Factor (VEGF) concentration as compared to PRP or whole blood.

According to a further specific embodiment, the serum fraction is characterized by a decreased PDGF-AB, PDGF-BB and TGF beta-1 as compared to PRP or whole blood.

According to a further specific embodiment, said isolated serum fraction of PRF comprises a reduced content of red blood cells, platelets or fibrinogen as compared to whole blood or a reduced content of fibrin as compared to said plasma.

According to a further specific embodiment, said isolated serum fraction of PRF has a non-inflammatory blood factor profile and a non-differentiating but cell proliferating profile on osteoblasts.

Preferably, the serum fraction is characterized by the depletion or reduction of two, three, four, or all five of these depleted factors, e.g. wherein one of these is at least SDF-1. The depletion, i.e. decrease or reduction of the specific factors, is typically determined, if the content or concentration is less than 50%, or less than 40%, or less than 30%, or less than 20%, or less than 10%, or less than 5% (w/w) of the concentration or content as compared to PRP or whole blood, e.g. measured by a proteome profiler, ELISA or similar assays.

Preferably, the serum fraction is depleted or free of red blood cells, e.g. substantially lacking red blood cells such that more than 75%, preferably more than 95% are removed, as compared to whole blood.

According to a specific embodiment, the serum fraction is further characterized by a reduced content of platelets as compared to whole blood, e.g. 10-fold, preferably 20-fold reduced. Upon separation of the coagel, the serum fraction typically contains less than 50*10⁹/L platelets, preferably less than 10*10⁹/L.

According to a specific embodiment, the serum fraction is further characterized by a reduced content of fibrinogen (e.g. determined by the fibrinogen plus fibrin content) as compared to whole blood, e.g. less than 20%, or less than 10%, or less than 5% (w/w). Upon separation of the coagel, the serum fraction typically contains less than 1.5 g/L fibrinogen+fibrin, preferably less than 0.5 g/L. Typically, the serum fraction is a clear or opaque solution without solid mass, e.g. without a fibrin clot visible to the naked eye.

According to a specific aspect, the serum fraction is freshly prepared and ready-to-use, optionally wherein the serum fraction is provided in an application device, preferably a syringe. Specific embodiments refer to an autologous serum fraction, i.e. a serum fraction prepared from blood or PRP of a single individual donor, which is ready-to-use for administration to the same individual. The serum fraction may be conveniently prepared in an appropriate preparation device suitable for aseptic collection of the blood, preparing the PRP, clotting the PRP (e.g. by actively initiating coagulation or by self-activation), separating the coagel and optionally further separating the solid PRF to isolate the serum fraction with or without the PRF fluid fraction, or to isolate the serum fraction from the PRF coagel. Preferably, the preparation device is suitable for aseptic collection of the blood, and preparing the PRP while it is self-activated whereupon a coagel is formed, and further separating the PRF coagel and obtaining the coagel supernatant together with the fluid fraction of the PRF. The isolated serum fraction may be produced in the application device in an aseptic way and may conveniently be directly and immediately administered to the individual, e.g. by an applicator aseptically connected to the preparation device, or by a separate application device or kit which allows the aseptic transfer of the prepared serum fraction to the application device and/or to administer the preparation to the individual.

According to the invention, the serum fraction is specifically provided for use in the manufacturing of an autologous pharmaceutical or medicinal product. Such product may be in the form of a pharmaceutical preparation or a medical device preparation.

Specifically, the serum fraction is provided for the treatment of the serum fractions donor. Specifically, the autologous use of the serum fraction is preferred.

In an embodiment, the invention provides for a medicinal use for plastic, reconstructive or regenerative medicinal use, preferably for use in orthopedic, surgical and/or cosmetic treatment.

In general terms, the invention, in a particular embodiment, provides for the serum fraction of the invention for medical use. Accordingly, the invention further refers to a method of treating a patient in need thereof with an effective amount of the serum fraction. Such effective amount is typically an amount sufficient to treat, repair or augment cells or tissue, e.g. local or topical treatment at a target site in need of cell proliferation or regeneration, e.g. skin, a wound, an injury, an incision, or a surgical site. In a preferred embodiment, the serum fraction is provided for use in the treatment of a patient suffering from or having suffered from bone ischemia or a related bone disease, including bone necrosis, osteoarthrosis or osteoarthritis, or other degenerative bone disease. In a preferred embodiment, the serum fraction is provided for use in facilitating or accelerating the propagation of bone tissue cells and thereby bone tissue regeneration after bone ischemia, or for use in the treatment of bone ischemia or any related disease or a disease which is a consequence of bone ischemia or a disease mediated by bone ischemia.

Accordingly, the invention further provides for a method for the treatment of a patient suffering from or having suffered from osteoarthritis, osteoarthrosis, bone necrosis, bone ischemia, or a disease as defined herein, comprising the steps of administering a serum fraction of the invention to said patient.

In an embodiment, the serum fraction can be used for treating a patient suffering from osteonecrosis, e.g. femoral head, Kohler I and II, Perthes, Osgood-Schlatter, or Scheuermann disease, osteoarthritis, osteoarthrosis, bone necrosis, tendinosis, e.g. tennis elbow, plantar fasciitis, or jumper's knee, critical limb ischemia, Buerger's disease, impingement syndrome, e.g. of the shoulder or the hip, or a patient undergoing treatment for dermal filling, rejuvenation of the nasolabial crest or any other facial wrinkles, bone grafting or implantation, preferably by culturing and administering cells, preferably patient's cells, in these diseases. Culturing may be carried out in vitro, optionally on cells present in or on a tissue or organ or partial organ taken out form a subject (ex vivo). The serum fraction of the invention would preferably induce proliferation after ischemia on explants of human osteonecrotic material. Preferably, when the serum fraction is mixed with bone grafts in vitro or in situ, the regeneration of bone material could be determined.

In a preferred embodiment, the invention provides for a method for facilitating or promoting the propagation of bone tissue cells and thereby bone tissue regeneration comprising the steps of administering the serum fraction of the invention to a bone tissue under or having subjected to bone ischemia, optionally to a patient suffering from or having suffered from osteoarthritis, osteoarthrosis, bone necrosis or bone ischemia.

In an embodiment the invention relates to the cell culture as defined above wherein the SPRF is prepared by a method as defined herein or above.

Preferably, said cell culture

does not comprise fetal bovine serum (FBS) or fetal calf serum (FCS), and does not comprise platelet rich plasma (PRP) and does not comprise any other growth factor either, only those which are present in the SPRF.

Preferably, said isolated serum fraction is depleted in a growth factor selected from the group consisting of PDGF-AB, PDGF-BB and TGF beta-1 as compared to platelet rich plasma (PRP).

Preferably said cells are mammalian cells, preferably selected from the group consisting of stem cells, epithelial cells, periosteogeneic cells, angiogenic cells, stromal cells, mesenchymal cells, osteoprogenitor cells and bone cells.

In a preferred embodiment said medium in the cell culture comprises 2-20% (v/v), preferably 5-15% (v/v), highly preferably 8 to 12% (v/v) or about 10% (v/v) SPRF and besides SPRF, said medium comprises no FBS (FCS) and no other serum derived product or supplement and preferably no other growth factors; wherein preferably the medium is a derivative of DMEM which differs from DMEM in that it is supplemented with 2-20% (v/v), preferably 5-15% (v/v), highly preferably with 8 to 12% (v/v) or about 10% (v/v) SPRF and comprises no other serum derived product or supplement and no other growth factors.

Preferably the cells in the culture are MSCs that are contacted or maintained in contact with SPRF, preferably for until at least a time-period when osteoblast direction differentiation occurs, preferably for until at least a time-period when the expression of at least one, preferably two or at least two osteoblast specific marker gene(s) is/are increased in a medium supplemented with SPRF.

Preferably the cells are chondrocytes and

SPRF is added to the culturing medium of chondrocytes.

In a still further embodiment, culturing of the cells can be carried out in vivo wherein the level of SPRF is maintained in vivo and thus the SPRF can be considered as an in vivo cell culture supplement or additive.

In an embodiment the cell culture comprising SPRF is prepared in vitro and then administered in vivo. Administration may follow the preparation of the cell culture, i.e. the addition of SPRF as a cell culture additive, and may be carried out either after propagation of the cells in the cell culture or shortly or even immediately after preparation of the cell culture.

In an embodiment the administration of the culture to the subject is carried out after at least 1, 2, 3, 4 or 5 days of culturing of the cells including propagation thereof, or after at least 5, 6 or 7 days or after at least 1 week, 2 weeks, 3 weeks or 4 weeks of culturing of the cells including propagation thereof.

In another embodiment the administration of the culture to the subject is carried out within 2 days or 1 day of culturing of the cells including at least maintaining thereof, or within 5, 6, 7 or 8 hours or within 1 hour, 2 hours, 3 hours or 4 hours of culturing of the cells including at least maintaining thereof, or even within 45 minutes, within 30 minutes or within 15 minutes after preparation of the cell culture, i.e. addition of SPRF as a cell culture additive.

In a preferred embodiment, the invention provides for the use of the autologous serum fraction, e.g. collecting a blood sample from a patient who is suffering from or having suffered from a disorder or disease condition, and to whom the preparation is administered to.

According to the invention, there is provided a method of promoting in vitro proliferation of cells by contacting a serum fraction of the invention with said cells and incubating said cells for a period of time sufficient to promote cell growth or regeneration, specifically wherein the cells are epithelial cells, stem cells or bone cells, e.g. osteocytes, osteoclasts, osteoblasts, or bone marrow derived cells such as mesenchymal stem cells and progenitor cells derived from them. Such in vitro treatment is specifically useful for preparing autogenous bone material or allografts.

Stem cells can be for example, as defined herein, adult stem cells, optionally “somatic stem cells” or “tissue stem cells”. Preferably stem cells are selected from the group consisting of hematopoietic stem cells, mammary stem cells, intestinal stem cells, mesenchymal stem cells, endothelial stem cells, neural stem cells, olfactory adult stem cells, neural crest stem cells, testicular cells.

In a preferred embodiment, the serum fraction is provided for in vitro use as a cell culture additive.

In a preferred embodiment, the serum fraction is provided to prepare bones or implants, e.g. metal implants, specifically by surface treatment or coating. For example, the serum fraction is used in preparing dental bone graft support.

Specific treatment methods according to the invention—either in vitro or in vivo—would refer to restoring the proliferation capacity of post-ischemic bone, effectively promoting vascularization and/or angiogenesis in regenerating tissue, or promoting the migration and/or infiltration of endogenous wound healing component such as periosteogeneic cells, angiogenic cells, stromal cells, mesenchymal cells, osteoprogenitor cells, osteoblasts, osteoclasts, or platelets. Specific treatment methods refer to bone, periosteum, tendon, muscle, fascia, nerve tissue, vascular tissue, and combinations thereof.

The serum fraction can be administered alone, or in combination or conjunction with either another agent or any other therapeutic treatment used in the indication, e.g. used to treat patients suffering from osteoarthritis, osteoarthrosis, bone necrosis, or bone ischemia or a patient in need of cultured cells, e.g. proliferated cells, in particular chondrocytes and/or mesenchymal stem cells. Thus, the serum fraction can be administered in combination or conjunction with cultured cells, in particular chondrocytes and/or mesenchymal stem cells.

According to the invention there is further provided a pharmaceutical preparation comprising the serum fraction and a pharmaceutically acceptable carrier and preferably a population or culture of cells.

Preferably, the pharmaceutical preparation further comprises an additional active substance and/or device to promote wound healing, cell proliferation or regeneration.

Preferably, the additional active substance is a hydrogel, a tissue sealant or an active component thereof, e.g. a gellifying agent which forms a hydrogel upon contact with the serum fraction of the invention, a tissue sealant component comprising fibrinogen and/or collagen, and/or a tissue sealant component comprising thrombin or prothrombin in combination with a prothrombin activator to generate thrombin. Preferably, the device is a solid or semi-solid or gel-like biomaterial suitable for use in humans (resorbable or non-resorbable), e.g. a bone graft material, e.g. including autogenous bone material, allografts, such as demineralized freeze-dried bone material, or alloplasts such as hydroxyapatite and tricalcium phosphate of synthetic or natural origin.

Preferably, the pharmaceutical preparation provided is ready-to-use, e.g. contained in an application device, in particular a syringe.

According to the invention, there is further provided an application kit comprising the components

a. the serum fraction of the invention; and

b. an application device, preferably a syringe.

Preferably, the kit may include further components or combinations, e.g. as a further component

c. a bone graft material, a gelling agent, a tissue sealant or an active component thereof; and

d. optionally a device for mixing the components a. and c. to obtain a mixture ready for application.

Chondrocyte Proliferation

In a preferred embodiment the invention relates to a use of a serum fraction of platelet rich fibrin (SPRF) prepared from whole blood obtained from one or more donor subject, for increasing proliferation rate of chondrocytes in vitro. Preferably, chondrocytes are dedifferentiated. Preferably, the use of SPRF does not redifferentiate chondrocytes from the dedifferentiated state.

Preferably, SPRF is obtained by
providing whole blood obtained from the one or more donor subject(s),
centrifuging whole blood at 1000 to 4000 g (or in the ranges as disclosed herein) for 2 to 20 minutes (or in a range as disclosed herein) within 4 minutes from blood collection, thereby providing a fibrin clot (platelet rich fibrin),
pressing or squeezing the fibrin clot to obtain the SPRF as an exudate or releasate thereof.

Preferably, said SPRF comprises less bFGF and/or less G-CSF than PRP and wherein said

SPRF comprises less pro-inflammatory factors than PRP, said pro-inflammatory factor(s) being selected from the group comprising at least IL-6, IL-8, IL-12, TNF-α.

Preferably, the SPRF does not redifferentiate chondrocytes from the dedifferentiated state or provides a lesser extent of redifferentiation than PRP, preferably measured by the col II/col I gene expression ratio.

The invention also relates to SPFR for use in transplantation or implantation of chondrocytes into a patient in need thereof, wherein said SPRF has been prepared from whole blood obtained from a donor subject and wherein said SPRF is used for increasing proliferation rate of chondrocytes to be transplanted or implanted to said patient. Preferably, said SPRF does not redifferentiate chondrocytes from the dedifferentiated state or provides a lesser extent of redifferentiation than PRP, preferably measured by the col II/col I gene expression ratio. Preferably, SPRF does not change the differentiation state of chondrocytes in hypoxia or normoxia.

In a preferred embodiment the SPRF for use according to the invention is obtained by
providing whole blood obtained from the one or more donor subject(s),
centrifuging whole blood at 1000 to 4000 g (or in a range as disclosed herein) for 2 to 20 minutes (or in a range as disclosed herein) within 4 minutes from blood collection, thereby providing a fibrin clot (platelet rich fibrin),
pressing or squeezing the fibrin clot to obtain the SPRF as an exudate or releasate thereof.

In a further preferred embodiment said SPRF comprises less bFGF and/or less G-CSF than PRP and wherein said SPRF comprises less pro-inflammatory factors than PRP, said pro-inflammatory factor(s) being selected from the group comprising at least IL-6, IL-8, IL-12, TNF-α.

In a preferred embodiment the SPRF obtained from a donor subject is used for increasing proliferation rate of chondrocytes in vitro before transplantation thereof, wherein preferably SPRF is applied in the cell culture in a concentration between 1-25% or 2-20%, preferably 5 to 15%, highly preferably 8 to 12% or in particular about 10%, wherein the percentage of concentration is given in v/v %. The same concentration range may be applied in liquid pharmaceutical preparations or in gel matrix preparations.

In a preferred embodiment the patient to be treated is a subject with cartilage failure, osteoarthritis, cartilage damage, cellular matrix linkage rupture, chondrocyte protein synthesis inhibition, and/or chondrocyte apoptosis, a condition requiring cartilage regeneration in particular cartilage ulcer, osteoarthritis or traumatic cartilage loss, a condition requiring subchondral bone regeneration such as osteoarthritis, Ahlback's disease or osteochondral lesions.

Preferably, the patient is a subject in need of cartilage repair, preferably articular cartilage repair, e.g. cartilage replacement therapy.

Preferably, the patient is a subject with cartilage failure, osteoarthritis, cartilage damage, cellular matrix linkage rupture, chondrocyte protein synthesis inhibition, and chondrocyte apoptosis.

In an embodiment the chondrocyte transplantation is autologous transplantation.

In a further embodiment the chondrocyte transplantation is heterologous transplantation.

In a preferred embodiment the donor subject is identical with the patient.

In a preferred embodiment the donor subject is different from the patient. Preferably, the age of the donor subject is below 50 years, preferably below 40 years, more preferably below 35 or 30 years. In a preferred embodiment the age of the patient is above 50 years or above 55 years or above 60 years.

In an embodiment the chondrocytes are transplanted into the patient and SPRF is administered to the patient to the same site as chondrocytes.

In an embodiment the SPRF is administered to the patient to the same site as chondrocytes essentially simultaneous with or after chondrocyte transplantation.

Preferably, SPRF is administered to the patient by injection or by matrix assisted transplantation.

In a preferred embodiment, SPRF obtained from a donor subject is used for increasing proliferation rate of chondrocytes in vitro before transplantation thereof.

Preferably, the chondrocytes are dedifferentiated.

Preferably, SPRF does not redifferentiate chondrocytes from the dedifferentiated state.

In an embodiment SPRF for use according to the invention is further provided in a pharmaceutical preparation comprising the SPRF and a pharmaceutically acceptable carrier.

Preferably, the pharmaceutical preparation further comprises an additional active substance and/or device to promote chondrocyte proliferation.

In a preferred embodiment, the pharmaceutically acceptable carrier is a matrix. If the matrix further promotes or facilitates chondrocyte transplantation it may be considered as an additional active substance. Preferably, the matrix is a hydrogel, a tissue sealant or an active component thereof, e.g. a gellifying agent which forms a hydrogel upon contact with the serum fraction of the invention, a tissue sealant component comprising fibrinogen and/or collagen, and/or a tissue sealant. In an other embodiment the matrix is a glycosaminoglycan, e.g. hyaluronic acid or hyaluronan.

In a further embodiment the SPRF is provided in a device for introducing SPRF into the site of cartilage repairs. In particular, the device is a solid or semi-solid or gel-like biomaterial suitable for use in humans (resorbable or non-resorbable).

In a further embodiment the device is an application device, in particular a syringe.

The invention also relates to method for increasing proliferation rate of dedifferentiated chondrocytes comprising contacting a serum fraction of platelet rich fibrin (SPRF) with said chondrocytes preferably in a medium, said SPRF being prepared from whole blood obtained from one or more donor subject(s).

Preferably in this method said SPRF is obtained by
providing whole blood obtained from the one or more donor subject(s),
centrifuging whole blood at 1000 to 4000 g (or in a range as disclosed herein) for 2 to 20 minutes (or in a range as disclosed herein) within 4 minutes from blood collection, thereby providing a fibrin clot (platelet rich fibrin),
pressing or squeezing the fibrin clot to obtain the SPRF as an exudate or releasate thereof.

Preferably in this method said SPRF comprises less bFGF and/or less G-CSF than PRP and wherein said SPRF comprises less pro-inflammatory factors than PRP, said pro-inflammatory factor(s) being selected from the group comprising at least IL-6, IL-8, IL-12, TNF-α.

In a preferred embodiment said method is a method for treatment of a patient in need of cartilage repair,

in particular articular cartilage repair and/or cartilage replacement therapy, preferably articular cartilage repair,

or in more particular the patient is a subject with cartilage failure, osteoarthritis, cartilage damage, cellular matrix linkage rupture, chondrocyte protein synthesis inhibition, and/or chondrocyte apoptosis, a condition requiring cartilage regeneration in particular in cartilage ulcer, osteoarthritis or traumatic cartilage loss, a condition requiring subchondral bone regeneration such as osteoarthritis, Ahlback's disease or osteochondral lesions,

wherein the SPRF is administered to said patient to or at the site of cartilage injury wherein SPRF is contacted with the dedifferentiated chondrocytes.

Preferably SPRF is administered to the patient by injection or by matrix assisted (induced) transplantation. Transplantation and implantation is used herein essentially interchangeably, in case of implantation the emphasis being on the implantation step, if cells are implanted, and not on the origin of cells.

In a preferred embodiment said method is a method of transplantation or implantation of chondrocytes into a patient in need thereof, wherein said SPRF is an SPRF prepared from whole blood obtained from a donor subject and wherein said SPRF is contacted with the dedifferentiated chondrocytes to be transplanted or implanted to said patient in vitro, to use for increasing proliferation rate of said chondrocytes.

Preferably, the patient is a subject in need of cartilage repair, in particular articular cartilage repair and/or cartilage replacement therapy, preferably articular cartilage repair,

or in more particular the patient is a subject with cartilage failure, osteoarthritis, cartilage damage, cellular matrix linkage rupture, chondrocyte protein synthesis inhibition, and/or chondrocyte apoptosis, a condition requiring cartilage regeneration in particular in cartilage ulcer, osteoarthritis or traumatic cartilage loss, a condition requiring subchondral bone regeneration such as osteoarthritis, Ahlback's disease or osteochondral lesions.

Preferably, said transplantation or implantation method comprises the following steps
culturing the condrocytes to proliferate or to expand,
introducing the proliferated chondrocytes into the patient.

The chondrocyte transplantation may be autologous transplantation or heterologous transplantation.

Preferably, said transplantation or implantation method comprises the following steps
chondrocytes obtained from the donor subject are contacted with the SPRF,
the chondrocytes are cultured to proliferate,
the proliferated chondrocytes are introduced into the patient.

In heterologous transplantation the donor subject is different from the patient, and preferably the age of the donor subject is below 50 years, preferably below 40 years, more preferably below 35 or 30 years.

In a preferred embodiment the age of the patient is above 50 years or above 55 years or above 60 years.

In an embodiment transplantation is autologous transplantation,

wherein said method comprises the steps of
chondrocytes obtained from the donor subject are contacted with the SPRF,
the chondrocytes are cultured to proliferate,
the proliferated chondrocytes are reintroduced into the patient who is identical with the donor subject.

In a preferred embodiment the chondrocytes are transplanted into the patient and SPRF is administered to the patient to the same site as chondrocytes.

Preferably, SPRF is administered to the patient to the same site as chondrocytes essentially simultaneous with or after chondrocyte transplantation.

In an embodiment simultaneous transplantation is carried out by mixing SPRF and a culture of chondrocytes.

Preferably, SPRF is administered to the patient by injection or by matrix assisted transplantation.

Preferably, SPRF does not redifferentiate chondrocytes from the dedifferentiated state or provides a lesser extent of redifferentiation than PRP, preferably measured by the col II/col I ratio.

Preferably, upon culturing of the chondrocytes, e.g. in the in vitro step, SPRF is applied to the cell culture in a concentration between 1 to 25% or 2-20%, preferably 5 to 15%, highly preferably 8 to 12% or in particular about 10%, wherein the percentage of concentration is given in v/v %.

In an embodiment SPRF for use according to the invention is further provided in a pharmaceutical preparation comprising the SPRF and a pharmaceutically acceptable carrier.

In a preferred embodiment, the pharmaceutical preparation further comprises an additional active substance and/or device to promote chondrocyte proliferation.

In a preferred embodiment, the pharmaceutically acceptable carrier is a matrix. If the matrix further promotes or facilitates chondrocyte transplantation it may be considered as an additional active substance. Preferably, the matrix is a hydrogel, a tissue sealant or an active component thereof, e.g. a gellifying agent which forms a hydrogel upon contact with the serum fraction of the invention, a tissue sealant component comprising fibrinogen and/or collagen, and/or a tissue sealant.

In a further embodiment the SPRF is provided in a device for introducing SPRF into the site of cartilage repairs. In particular, the device is a solid or semi-solid or gel-like biomaterial suitable for use in humans (resorbable or non-resorbable).

In a further embodiment the device is an application device, in particular a syringe.

In a particular embodiment the syringe is a device which is suitable to obtain and coagulate blood. In a particular embodiment the syringe is a device as disclosed in WO 2017/093838.

MSC Proliferation

In a preferred embodiment the invention further relates to a method for use of serum fraction of platelet rich fibrin (SPRF) for increasing mesenchymal stem cell (MSC) proliferation rate in vitro, ex vivo or in vivo wherein said MSCs maintain their potential to differentiate into several cell types.

The invention relates to a use of serum fraction of platelet rich fibrin (SPRF) for increasing MSC proliferation rate in vitro, ex vivo or in vivo wherein said MSCs maintain their potential to differentiate into several cell types.

Preferably, in the method or use of the invention the MSCs are contacted or maintained in contact with SPRF for at least 5 days, preferably for at least 8 days or at least 10 days.

In a particular embodiment there is provided a method of promoting in vitro proliferation of cells by contacting a serum fraction of the invention with said cells and incubating said cells for a period of time sufficient to promote cell growth or regeneration, wherein the cells are mesenchymal stem cells or progenitor cells derived from them. Such in vitro method may also be useful for preparing autogenous bone material or allografts.

In an in vitro method or use the MSCs are maintained in culture. In an embodiment a medium for culturing mammalian cells e.g. an MSC culturing medium supplemented with SPRF is applied. MSC culturing medium normally comprises a carbon source e.g. sugar source e.g. glucose. According to the invention the medium comprises SPRF and comprises no further serum and/or no further serum substitute and/or no further serum derived product or supplement. In particular the medium according to the invention does not comprise fetal bovine (calf) serum (FBS or FCS) and does not comprise platelet rich plasma (PRP). In a particular embodiment the medium according to the invention does not comprise any further growth factor (only those which are present in the SPRF).

Preferably, in a method for using SPRF for selectively increasing MSC proliferation rate in vitro said differentiated MSCs maintain their potential to differentiate into several cell types. Preferably MSCs are obtained from a subject, said method comprising
providing SPRF,
adding SPRF to a pool of MSCs in medium,
allowing MSCs to proliferate.

Preferably, in the method or use of the invention the MSCs are contacted or maintained in contact with SPRF for until at least a time-period when osteoblast direction differentiation occurs, preferably for until at least a time-period when the expression of at least one, preferably two or at least two osteoblast specific marker gene(s) is/are increased in a medium supplemented with SPRF, preferably SPRF having the concentration range given herein, highly preferably with 10% (v/v) SPRF (and comprising no other serum or serum derived product or supplement) when compared with 10% (v/v) FCS supplemented medium.

Preferably expression of one or both of the following osteogenic marker genes is increased:

COL1A1 and ALPL, wherein preferably
COL1A1 expression is increased at least 5 fold (by 400%), preferably at least 6 fold or 7 fold,
ALPL expression is increased at least 5 fold (by 400%), preferably at least 6 fold or 7 fold, when compared to 10% (v/v) FCS supplemented medium.

In a preferred embodiment the MSCs so proliferated are administered to a subject. In a preferred embodiment the subject is a patient in need of bone or cartilage regeneration or repair.

In a preferred embodiment the patient is treated for a condition wherein the level of differentiable MSCs is low, preferably pathogenically low, preferably said condition being selected from impaired bone tissue, spongy bone tissue defect, osteonecrosis, osteoarthrosis or osteoarthritis.

In a preferred embodiment the patient is in need of bone tissue regeneration.

Preferably the patient is suffering from osteoarthritis or osteoarthrosis, preferably osteoarthritis or osteoarthritis of a joint. Preferably the patient is a mammalian or human subject.

In an ex vivo method or use the MSCs are present in or on a tissue and so cultured or maintained in culture.

In an embodiment the tissue is an explant. In an embodiment the tissue is an artificial tissue. In an embodiment the tissue is an explant, e.g. a bone explant. The bone explants may be e.g. subchondral bone pieces or explants obtained by osteotomy. In an embodiment the tissue is an artificial tissue, e.g. a bone graft or a joint or cartilage graft.

In an embodiment an MSC culturing medium for maintaining or culturing a tissue ex vivo is a medium for culturing mammalian cells e.g. a medium for culturing MSCs supplemented with SPRF. MSC culturing medium normally comprises a carbon source e.g. sugar source e.g. glucose, a glutamine source and pyruvate. According to the invention the medium comprises SPRF and no further serum and/or no further serum substitute and/or no further serum derived product or supplement. In particular the medium according to the invention does not comprise fetal bovine (calf) serum (FBS or FCS) and does not comprise platelet rich plasma (PRP). In a particular embodiment the medium according to the invention does not comprise any further growth factor (only those which are present in the SPRF).

Preferably MSCs are obtained from a subject, said method comprising
providing SPRF,
adding SPRF to a pool of MSCs present in an ex vivo tissue or on an explant,
allowing MSCs to proliferate.

In the method or use of the invention the MSCs are incubated in the presence of SPRF for at least 5 days, preferably for at least 8 days or at least 10 days.

In an embodiment the MSCs are bone marrow derived mesenchymal stem cells (BM-MSCs or bone marrow stromal stem cells).

In an embodiment the MSCs are adipose derived mesenchymal stem cells (AD-MSCs).

In an embodiment the medium is as defined above or a medium as disclosed herein.

In a preferred embodiment the tissue or explant on which MSCs are so proliferated is administered to a subject. In a preferred embodiment the tissue or explant is a graft to be implanted into the subject.

In a preferred embodiment the subject is a subject in need of bone or cartilage regeneration or repair. Preferably the subject is suffering from osteoarthritis or osteoarthrosis, preferably osteoarthritis or osteoarthritis of a joint. Preferably the subject is a mammalian or human subject.

In an embodiment the MSCs are mammalian MSCs, preferably human MSCs (hMSCs).

The invention also relates to a method for use of serum fraction of platelet rich fibrin (SPRF) for increasing mesenchymal stem cell (MSC) proliferation rate in vitro wherein said MSCs maintain their potential to differentiate into several cell types.

The invention relates to a method for use of serum fraction of platelet rich fibrin (SPRF) for increasing MSC proliferation rate in vitro wherein said MSCs maintain their potential to differentiate into several cell types. Preferably the expression of at least one or two, preferably two or at least two osteoblast differentiation factors show increased expression after an appropriate period of time, preferably at or after 5 days culturing. Preferably the osteoblast factors are COL1A1 and ALPL.

In the method or use of the invention the MSCs are incubated in the presence of SPRF for at least 5 days, preferably for at least 8 days or at least 10 days.

In a preferred embodiment the MSCs are obtained from a subject. Preferably the subject is a mammalian subject, more preferably a human subject.

In an embodiment the MSCs are primary cells.

In an embodiment the MSCs are bone marrow derived mesenchymal stem cells (BM-MSCs or bone marrow stromal stem cells).

In an embodiment the MSCs are adipose derived mesenchymal stem cells (AD-MSCs).

In an embodiment the medium is as defined above or a medium as disclosed herein.

In a preferred embodiment the culture of mesenchymal stem cells is supplemented with 2-20% (v/v), preferably 5-15% (v/v), highly preferably with 8 to 12% (v/v) or about 10% (v/v) SPRF and comprises no other serum derived product or supplement and preferably no other growth factors.

In a preferred embodiment the culture medium used for increasing proliferation rate of mesenchymal stem cells comprises
one or more amino acid source, preferably at least glutamate source or a glutamate source only,
one or more salts, said salt being preferably selected from calcium chloride, potassium chloride, magnesium sulfate, sodium chloride and monosodium phosphate,
one or more sugar, preferably at least glucose or glucose only and optionally or if desired,
one or more vitamins preferably selected from folic acid, nicotinamide, riboflavin and B12,

wherein said medium comprises 2-20% (v/v), preferably 5-15% (v/v), highly preferably 8 to 12% (v/v) or about 10% (v/v) SPRF,

wherein said medium comprises no FBS (FCS) and no PRP and preferably no FGF, and/or

wherein preferably said medium comprises, besides SPRF, no other serum product and/or no other serum derived product or supplement and preferably no other growth factors.

The medium may comprise further additives e.g. buffer(s), antibiotic(s), selection agent(s), preservation agent(s) etc.

In a preferred embodiment the medium is a derivative of Dulbecco's modified Eagle's medium (DMEM) which differs from DMEM in that it is supplemented with 2-20% (v/v), preferably 5-15% (v/v), highly preferably with 8 to 12% (v/v) or about 10% (v/v) SPRF and comprises no other serum derived product or supplement and preferably no other growth factors.

In an embodiment the invention relates to an in vivo method or use wherein
SPRF is contacted with MSCs of a subject in vivo and
an appropriate level of SPRF is maintained for increasing mesenchymal stem cell (MSC) proliferation rate in vivo wherein said MSCs maintain their potential to differentiate into several cell types.

Preferably, SPRF is administered to the subject thereby contacting said SPRF with the MSCs present in said subject.

Preferably, no other serum derived product or supplement and preferably no other growth factors are administered to the subject besides SPRF.

Preferably, in the method or use of the invention the MSCs are maintained in contact with or in the presence of SPRF in vivo for at least 5 days, preferably for at least 8 days or at least 10 days.

Preferably, in the method or use of the invention the subject is in need of regeneration of cartilage and/or bone,

SPRF is administered to a site wherein it may be contacted with the bone or cartilage to be regenerated, and

MSCs present at the site of administration are contacted or maintained in contact with SPRF, and

SPRF level is maintained to proliferate MSCs for until at least a time-period when osteoblast direction differentiation occurs, preferably for until at least a time-period when the expression of at least one, preferably two or at least two osteoblast specific marker gene(s) is/are increased in a medium supplemented with SPRF, preferably SPRF having the concentration range given herein, highly preferably with 10% (v/v) SPRF (and comprising no other serum or serum derived product or supplement) when compared with 10% (v/v) FCS supplemented medium.

In a further preferred embodiment the MSCs present at the site of administration in the subject are MSCs propagated according to the present invention, preferably MSCs obtained from a subject, cultured and propagated in vitro and reintroduced or re-administrated to said subject.

Preferably expression of one or both of the following osteogenic marker genes is increased:

COL1A1 and ALPL, wherein preferably
COL1A1 expression is increased at least 5 fold (by 400%), preferably at least 6 fold or 7 fold,
ALPL expression is increased at least 5 fold (by 400%), preferably at least 6 fold or 7 fold,

when compared with 10% (v/v) FCS supplemented medium.

Preferably the subject is a mammalian subject, more preferably a human subject.

In an embodiment the MSCs are bone marrow derived mesenchymal stem cells (BM-MSCs or bone marrow stromal stem cells).

In an embodiment the MSCs are adipose derived mesenchymal stem cells (AD-MSCs).

Preferably, upon culturing the MSCs in contact with SPRF, expression of MSC-specific genes is maintained or MSC-specific genes remain intensely expressed. In a particular embodiment, the expression level of the following MSC-specific genes is unchanged or is increased by 1 to 150% in comparison with the same medium supplemented with the same concentration of FCS instead of SPRF or in comparison with the same medium supplemented with 10% of FCS instead of SPRF. Preferably, the expression level is measured by real time quantitative PCR (rt-qPCR). Preferably, the expression level is measured on or after 5 days as of starting the administration of SPRF or contacting the cells with SPRF. In particular the expression levels of the following MSC marker genes are increased: ALCAM (CD166), ITGB1, CD105, ANPEP. Thus the MSC type or features of the cells are maintained.

Preferably, the expression of hMSC-specific genes are increased after 5 days incubation in a medium supplemented with SPRF, preferably SPRF having the concentration range given above, preferably with 10% (v/v) SPRF (and comprising no other serum or serum derived product or supplement) when compared with 10% (v/v) FCS supplemented medium as follows:
ALCAM expression is increased at least 1.2 fold (by 20%), preferably at least 1.4 fold or 1.6 fold,
ITGB1 expression is increased at least 1.5 fold (by 50%), preferably at least 1.7 fold or 1.9 fold,
CD105 expression is increased at least 1.05 fold (by 5%), preferably at least 1.1 fold or 1.2 fold and
ANPEP expression is increased at least 1.1 fold (by 10%), preferably at least 1.2 fold or 1.3 fold,

preferably as confirmed by real time qPCR.

Alternatively, any one of the above markers is at least not decreased.

Preferably, no adipose differentiation occurs in the MSCs when MSCs are cultured in SPRF, preferably 10% (v/v) SPRF.

In a preferred embodiment the expression level of adipogenic (adipocyte) markers FABP4, PPARG and ADIPOQ expression, that are markers of adipogenic differentiation, is not increased by more than 1.3 fold (less than by 30%), preferably 1.2 fold (less than by 20%), more preferably 1.1 fold (less than by 10%) upon culturing according to the invention, preferably after 5 days or further culturing, in comparison with 10% (v/v) FCS supplementation.

However, an osteoblast direction differentiation occurs in the cells upon culturing according to the invention, preferably after 5 days or further culturing, in comparison with 10% (v/v) FCS supplementation.

In particular, the expression of at least one, preferably two osteoblast specific marker gene(s) is/are increased after 5 days incubation in a medium supplemented with SPRF, preferably SPRF having the concentration range given above, preferably with 10% (v/v) SPRF (and comprising no other serum or serum derived product or supplement) when compared with 10% (v/v) FCS supplemented medium as follows:
COL1A1 expression is increased at least 5 fold (by 400%), preferably at least 6 fold or 7 fold,
ALPL expression is increased at least 5 fold (by 400%), preferably at least 6 fold or 7 fold, and preferably
RUNX2 expression is at least not reduced or is increased at least 1.05 fold (by 5%), preferably at least 1.1 fold or 1.2 fold,

when compared with 10% (v/v) FCS supplemented medium,

preferably as confirmed by real time qPCR.

In an embodiment the BAX/BCL2 ratio was elevated at least 15, preferably at least 20 or 25 fold both in case of 10% (v/v) FCS+1 ng/mL bFGF supplement and in case of the medium as used in the present invention, in particular when 10% (v/v) SPRF supplementation was used, in comparison with 10% (v/v) FCS supplementation.

The invention also relates to a medium or a method for using a medium as disclosed herein in a culture for increasing proliferation rate of mesenchymal stem cells (MSCs) or for culturing MSCs as disclosed herein.

Thus, the invention also relates to a method for using SPRF as a cell medium supplement instead of PRP and FBS wherein said SPRF enhances the proliferation rate of human mesenchymal stem cells in vitro while phenotypical changes were not observed except that the levels of osteoblast markers are increased and differentiation potential of proliferated MSCs was maintained.

Said medium comprises SPRF as a supplement and as a serum-derived product. Preferably the medium does not comprise fetal bovine serum (FBS or fetal calf serum, FCS) and does not comprise platelet rich plasma (PRP) and preferably does not comprise FGF (e.g. bFGF) and preferably does not comprise any other growth factor either.

Preferably the medium comprising SPRF does not comprise any further serum product or serum derived product or supplement and preferably does not comprise any other growth factor besides those present in SPRF.

In a preferred embodiment the culture medium comprises
one or more amino acid source, preferably at least glutamate source or a glutamate source only,
one or more salts, said salt being preferably selected from calcium chloride, potassium chloride, magnesium sulfate, sodium chloride and monosodium phosphate,
one or more sugar, preferably at least glucose or glucose only and optionally or if desired,
one or more vitamins preferably selected from folic acid, nicotinamide, riboflavin and B12,

wherein said medium comprises 2-20% (v/v), preferably 5-15% (v/v), highly preferably 8 to 12% (v/v) or about 10% (v/v) SPRF.

The medium may comprise further additives e.g. buffer(s), antibiotic(s), selection agent(s), preservation agent(s) etc.

In a preferred embodiment the medium is a derivative of Dulbecco's modified Eagle's medium (DMEM) which differs from DMEM in that it is supplemented with 2-20% (v/v), preferably 5-15% (v/v), highly preferably with 8 to 12% (v/v) or about 10% (v/v) SPRF and comprises no other serum derived product or supplement and preferably no other growth factors.

In an embodiment of the invention the SPRF selectively increases mesenchymal stem cell (MSC) proliferation rate which means that proliferation rate of mesenchymal stem cells (MSC) increases at a higher extent than at least one other adult stem cell type, e.g. that of hematopoietic stem cells.

The invention also relates to the use of serum fraction of platelet rich fibrin (SPRF) obtained from a donor subject to test selective increase of MSC proliferation rate in vitro. Preferably, MSCs are undifferentiated cells with the potential to differentiate into several cell types.

Preferably, in the use of SPRF or in the method of the invention MSCs doesn't differentiate in vitro into adipocyte direction.

In an embodiment, the SPRF may be obtained from a blood sample from a single donor subject or from multiple donor subjects and mixed together to obtain a single blood sample. Alternatively SPRF obtained from multiple donors can be mixed together.

According to a specific aspect, the SPRF is obtained from venous blood collected from a single donor.

In a preferred embodiment the donor is the patient to whom, once proliferated, the MSCs are reintroduced.

The invention also relates to the use of SPRF in stem cell therapy preferably in MSC therapy, wherein SPRF obtained from a donor subject is used to increase proliferation rate of the patient's in vitro expanded MSCs. Preferably, the donor subject of the SPRF is the same subject as the patient to be treated with MSCs. Preferably, the donor subject of the SPRF is the same subject as the patient from whom the MSCs are obtained.

Preferably, with the use of SPRF, MSCs do not differentiate in vitro. Preferably, however, osteoblast markers appear on the MSCs on a given period of time e.g. 5 days culturing.

The invention also relates to the use of SPRF in stem cell therapy wherein the patient's own cells are proliferated in situ (in vivo) or in vitro or ex vivo.

Preferably, the patient is a subject in need of bone tissue regeneration. Preferably the patient is a subject with spongy bone tissue defect, osteonecrosis, osteoarthrosis or osteoarthritis.

In an application the lack of differentiable MSCs in the subchondral/spongy bone can be cured/treated by stem cell therapy or stem cell transplantation.

In an application the MSC transplantation is autologous transplantation followed by ex vivo multiplication.

In a preferred application the ex vivo multiplication is carried out in a medium comprising SPRF as the patient's own blood separation product.

In a preferred application transplantation is not needed, because the proliferation of resident MSCs can be enhanced.

In a preferred embodiment the used therapy comprises the proliferation of MSCs resident in a tissue of a patient wherein the SPRF is administered to the tissue of said patient to enhance proliferation of MSCs in said tissue. Preferably in said tissue the level of differentiable MSCs is low, preferably pathogenically low, preferably said condition being selected from impaired bone tissue, spongy bone tissue defect, osteonecrosis, osteoarthrosis or osteoarthritis.

In an application the SPRF is administered to the patient to the same site as in vitro expanded own MSCs, essentially simultaneous with or after MSC transplantation.

Preferably the tissue is impaired bone tissue or cartilage tissue.

Preferably, SPRF is administered to the patient by matrix assisted transplantation.

The invention also relates to a method of treatment wherein MSCs are obtained from said patient and the patient's own cells are proliferated in vitro, wherein the MSC transplantation is autologous transplantation followed by ex vivo expansion.

The invention also relates to a method of treatment of a patient in a stem cell therapy wherein SPRF obtained from a donor subject is used to increase proliferation rate of the patient's MSCs expanded ex vivo,

wherein the MSCs so proliferated maintain their undifferentiated character with the potential to differentiate into several cell types. Preferably, the MSCs show increased expression of at least one or two, preferably two osteoblast markers, preferably COL1A1 and/or ALPL.

In a preferred embodiment the donor subject of blood from which the SPRF is obtained is identical with the patient.

In a preferred embodiment the patient is treated for a condition wherein the level of differentiable MSCs is low, preferably pathogenically low, preferably said condition being selected from impaired bone tissue, spongy bone tissue defect, osteonecrosis, osteoarthrosis or osteoarthritis.

In a preferred embodiment the patient is in need of bone tissue regeneration.

Preferably, in the treatment subchondral and/or spongy bone is treated in a stem cell therapy or stem cell transplantation by MSCs proliferated using said SPRF.

Preferably said therapy comprises the proliferation of MSCs resident in a tissue of a patient wherein the SPRF is administered to the tissue of said patient to enhance proliferation of MSCs in said tissue, and

wherein the level of differentiable MSCs is low, preferably pathogenically low, preferably said condition being selected from impaired bone tissue, spongy bone tissue defect, osteonecrosis, osteoarthrosis or osteoarthritis.

Preferably the tissue is impaired bone tissue or cartilage tissue.

////In a further preferred embodiment in said treatment administration of SPRF is applied together with a method, wherein said method is an in vitro method and in step ii. the SPRF is added to a pool of MSCs is a culture medium, said medium comprises 2-20% (v/v), preferably 5-15% (v/v), highly preferably with 8 to 12% (v/v) or about 10% (v/v) SPRF and comprises no other serum derived product or supplement and preferably no other growth factors. Preferably, the MSCs so propagated are (for) reintroduction to a patient is a subject in need of bone tissue regeneration or the patient is a subject suffering in spongy bone tissue defect, osteonecrosis osteoarthrosis or osteoarthritis.

In an embodiment, SPRF is added to a pool of MSCs on a tissue or explant in a medium wherein said medium comprises 2-20% (v/v), preferably 5-15% (v/v), highly preferably with 8 to 12% (v/v) or about 10% (v/v) SPRF and comprises no other serum derived product or supplement and preferably no other growth factors. In a preferred embodiment the ex vivo tissue is a bone or cartilage graft and said graft is reintroduced into a patient in need thereof, wherein said patient is a subject in need of bone tissue regeneration or the patient is a subject suffering in spongy bone tissue defect, osteonecrosis osteoarthrosis or osteoarthritis.

In a further preferred embodiment said method is an in vivo method, wherein
SPRF is contacted with MSCs of a subject in vivo and
an appropriate level of SPRF is maintained for increasing mesenchymal stem cell (MSC) proliferation rate in vivo wherein said MSCs maintain their potential to differentiate into several cell types and wherein upon proliferation of MSCs expression of one or both of the following osteogenic marker gene/s is/are increased: COL1A1 and ALPL, and wherein

no other serum derived product or supplement and preferably no other growth factors are administered to the subject besides SPRF, and

the MSCs are maintained in contact with or in the presence of SPRF in vivo for at least 5 days.

In a preferred embodiment SPRF is contacted with MSCs of a subject in vivo by administering SPRF to a site of said subject wherein it may be contacted with the bone or cartilage to be regenerated, and MSCs present at the site of administration are contacted or maintained in contact with SPRF for at least 5 days.

In a variant upon proliferation of MSCs expression of one or both of the following osteogenic marker gene/s is/are increased: COL1A1 and ALPL.

In an embodiment SPRF is administered to the patient in a matrix.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Protocol of simulated ischemia and reperfusion in human bone explants. Bone tissue pieces were isolated at day 0 at total hip replacement procedures and kept in culture for 3 days. Cells were subjected to damage through oxygen glucose deprivation (OGD) for 7 hours on day 3, followed by the replacement of normal stem cell medium and a return of oxygen levels to normal. Serum fractions were added to the explanted cultures at just before OGD and replaced at medium changes when necessary until the end of the experiment. Cell viability was measured on either the 6th or the 9th days by replacing the tissues in a fresh well and thus measuring the cells on the bone matrix only.

FIGS. 2A to 2D. Effect of PRP treatment on bone explants after OGD. FIG. 2A and FIG. 2B show that neither PRP nor heparinized PRP has any effect on cell viability after 3 or 6 days reperfusion (n=24/group). FIG. 2D shows that increasing the concentration of PRP to the technically feasibly maximum level without affecting the native preparation has still no effect on the proliferation, and FIG. 2C shows that PRP activated by different ways such as adding Calcium, Calcium+Thrombin, or subjecting the preparation to 3 cycles of freezing and thawing was also without effect. Data are presented as average±SEM.

FIGS. 3A and 3B. Effect of SPRF on bone explants after OGD. FIG. 3A shows that there is no effect immediately after 7-hours OGD (n=18-24/group), but cells started to proliferate significantly better after 6 days. FIG. 3B shows the effect of SPRF-pretreatment when the serum fraction was present in the medium from day 0 (n=24/group). Data are presented as average±SEM, ** represents p<0.01, *** represents p<0.001.

FIGS. 4A and 4B. Constituents of serum fractions measured by the Proteome Profiler array. Protein levels are measured by the intensity of spots in arbitrary units, compared between SPRF and PRP. Data are split between FIG. 4A and FIG. 4B for legibility. Data are presented as average±SEM, n=3 subjects, each spot is measured in duplicates.

FIG. 5. Schematic comparison of exemplary isolation of Platelet rich plasma (PRP) and SPRF.

FIG. 6. Exemplary isolation of Platelet rich plasma (PRP)

FIGS. 7A and 7B. Multiplex array—protein quantification. FIG. 7A. Human blood donors; n=10, data represented as interquartile median. FIG. 7B. Human blood donors; n=6, data represented as interquartile median

FIG. 8. Multiplex array—protein quantification. SPRF or PRP pooled from 10 individual donors; data represented as interquartile median.

FIGS. 9A and 9B. Cell proliferation of chondrocytes from OA patients. * represents p<0.05.

FIGS. 10A-D. Col I gene expression of OA chondrocytes under conditions of normoxia and hypoxia.

FIGS. 11A-D. Col II gene expression of OA chondrocytes under conditions of normoxia and hypoxia. *** represents p<0.001.

FIGS. 12A-D. Index of cell differentiation (redifferentiation). The Col II/Col I ratio is indicative of the cell-cell differentiation (in dedifferentiated cell the redifferentiation) potential of cells. * represents p<0.05 and ** represents p<0.01.

FIGS. 13A-D. Matrix metalloproteinase 3 (MMP 3) gene expression of OA chondrocytes under conditions of normoxia and hypoxia.

FIGS. 14A-D. Matrix metalloproteinase 13 (MMP 13) gene expression of OA chondrocytes under conditions of normoxia and hypoxia. ** represents p<0.01.

FIGS. 15A and 15B. Proliferative effect of serum derivatives in isolated human chondrocytes. ** represents p<0.01 and *** represents p<0.001.

FIGS. 16A and 16B. FIG. 16A. Representation of the mean results from 3 different experiments with XTT assay after 8 days, performed in 96-well cell culture plate based monolayer culture with 2000 cells/well seeded, cultured in serum free DMEM (SF) or DMEM media supplemented with 10% SPRF, 10% PRP or 10% FCS. *** represents p<0.001. FIG. 16B. Pictures of OA chondrocyte cultures after 0, 4, 8 days with different media supplementation.

FIG. 17. Autologous chondrocyte implantation process scheme.

FIG. 18. Time-course effect of serum supplements on hMSCs. Subconfluent hMSCs were cultured in DMEM in the absence of supplement (col. 1 at day 2 and 5), 10% (v/v) of FCS (col. 2 at day 2 and 5) or 10% (v/v) FCS+1 ng/mL bFGF (col. 3 at day 2 and 5), or 10% (v/v) PRP (col. 4 at day 2 and 5) or 10% (v/v) SPRF (col. 5 at day 2 and 5). Results are presented as means of triplicate samples in three experiments±SD. *** represents p<0.001.

FIG. 19. Cell morphology of hMSCs in differently supplemented media, using phase contrast microscopy (magnification 10×). Cells were cultured in 10% (v/v) of FCS (upper row, left); 10% (v/v) FCS+1 ng/mL bFGF (upper row, right); 10% (v/v) PRP (lower row, left) or 10% (v/v) SPRF (lower row, right).

FIGS. 20A to 20D. Cell immunophenotypes of hMSCs cultured in differently supplemented media. Mesenchymal stem cells were cultured in 10% (v/v) of FCS ( [symbol] ) or 10% (v/v) FCS+1 ng/mL bFGF ( [symbol] ), or 10% (v/v) PRP (▪) or 10% (v/v) SPRF (▪) and stained with specific antibodies. In the first three pages of FACS diagrams (FIG. 20A, FIG. 20B and FIG. 20C) representative images of expression of hMSC markers CD90, CD105, and CD73, respectively, are shown. On the fourth page of FACS diagrams (FIG. 20D) representative images of expression of hematopoietic markers CD34, CD11b, CD19 and CD45 are shown. The fluorochromes applied were FITC (fluorescein isothiocyanate), Cy5 cyanine dye, APC (Allophycocyanin) and PE (Phycoerythrin), respectively.

FIGS. 21A-D. Gene expression analysis of differently supplemented hMSCs cultures. In these figures hMSC (FIG. 21A), adipocyte (FIG. 21B), osteoblast (FIG. 21C) and apoptotic (FIG. 21D) marker levels are shown in hMSC cultures after 5 days culturing. Mesenchymal stem cells were cultured in 10% (v/v) of FCS (background color, control) or 10% (v/v) FCS+1 ng/mL bFGF (col. 1), or 10% (v/v) PRP (col. 2) or 10% (v/v) SPRF (col. 3). On the Y axis the relative protein expression level ratio is presented, the base of comparison is the level in 10% FCS cultures (background color).

Data are presented as fold change values to the expression of hMSCs cultured in 10% (v/v) FCS-supplemented medium that was considered as the standard growing medium.

FIG. 22. Culture of human subchondral bone chips. Following 5 days incubation cells in SPRF show alike if not better viability increase as cells in FCS medium. Blood serum free medium did not induce proliferation of cells. Serum-free medium (◯), 10% (v/v) of FCS (Δ), 10% (v/v) SPRF (⋄).

FIG. 23. Histological analysis of hSBPs. Culturing hSBPs in 10% (v/v) SPRF supplemented medium for 5 days preserved bone marrow integrity as hematoxylin and eosin-stained sections (A) and Masson's trichrome sections (B) show. 10% (v/v) FCS supplementation for 5 days appeared less effective therein (E, F). That means, SPRF revealed higher level of hMSC accumulation (C) compared to FCS (G), and preserved better the local vasculature (D, H).

FIGS. 24A-E. Gene expression analysis of human subchondral bone chips. Relative gene expression level is shown on Y axis, compared to the values measured right after the bone chip explanation. Serum-free medium (◯), 10% (v/v) of FCS (Δ), 10% (v/v) SPRF (⋄).

FIG. 24A: demonstrates that hMSC markers did not change in average.

FIG. 24A1: ENG, FIG. 24A2: ITGB1, FIG. 24A3: ANPEP, FIG. 24A4: ALCAM

FIG. 24B: indicates that the hematopoetic cells were not induced, however they could be present in bone chips.

FIG. 24B1: CD34, FIG. 24B2: CD14, FIG. 24B3: PTPRC

FIG. 24C: shows that adipocytes were not induced in SPRF medium.

FIG. 24C1: PPARG, FIG. 24C2: FABP4, FIG. 24C3: ADIPOQ

FIG. 24D: the increase of the expression level of osteoblastic genes is demonstrated.

FIG. 24D1: COL1A1, FIG. 24D2: P4HA2, FIG. 24D3: ALPL, FIG. 24D4: RUNX2

FIG. 24E: presents the expression level of osteocytic genes.

FIG. 24E1: DMP1, FIG. 24E2: MEPE, FIG. 24E3: PDPN.

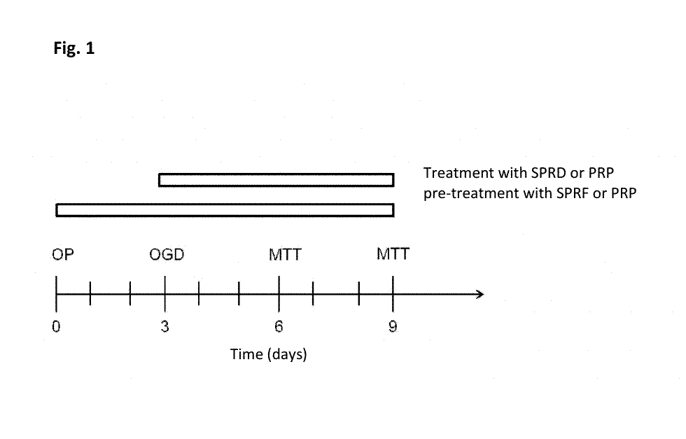

### DEFINITIONS

The term “clotting” as used herein in relation to blood coagulation is herein understood in the following way. Platelet activation and subsequent degranulation and aggregation play a pivotal role in blood clotting. Coagulation can be activated through the intrinsic or “contact activation pathway” which is initiated when blood coagulation factor XII comes into contact with negatively charged surfaces in a reaction involving high molecular weight kininogen and plasma kallikrein. FXII can be activated by so-called “contact activators”, e.g. the biological macromolecular constituents of the subendothelial matrix such as glycosaminoglycans and collagens, sulfatides, nucleotides, and other soluble polyanions or non-physiological material such as glass, or polymers, in particular artificial negatively charges surfaces, such as glass beads. Besides, the coagulation cascade supports the blood coagulation process. The coagulation cascade involves a series, i.e. cascade of reactions, in which a zymogen is activated, e.g. by enzymes supported by co-factors, to become an active enzyme that then catalyzes the next reaction in the reaction cascade, ultimately resulting in the formation of a fibrin clot, which strengthens the platelet aggregate. The zymogens are also known as coagulation factors or clotting factors.

As a result of coagulation activation, a blood clot is formed, which is herein referred to as a “coagel”. A coagel is specifically understood as the coagulated phase of blood, i.e. the soft, coherent, jelly-like mass resulting from the conversion of fibrinogen to fibrin mainly consisting of fibrin fibers associated to form a fibrin gel or clot. The coagel as described herein specifically is entrapping platelets and further components of coagulated plasma.

The coagel emanated from PRP is specifically understood as platelet rich fibrin (PRF) which may specifically include aggregated fibrin and blood cells, such as platelets, white blood cells, and/or red blood cells.

The coagel of PRF is herein understood to be composed of two fractions, the fluid fraction and the solid fraction, which may be physically separated to isolate the liquid phase and discard the solid mass.

Coagulation is specifically activated in a suitable container, such as a clot container or clot activating container, e.g. a tube. The container is suitably a glass or plastic container, with or without additional means to initiate or accelerate clotting, e.g. blood collection tubes generally used in the medical practice. In particular, the clot container does not contain anticoagulants, and is used without adding anticoagulants, so to support the clotting in situ. According to a specific embodiment, the clot container is suitably equipped with contact activating surfaces to activate the intrinsic coagulation pathway.

The term “platelet rich plasma” or PRP is herein understood as a volume of plasma that has a platelet concentration above baseline. Normal platelet counts in blood range between 150,000/microliter and 350,000/microliter. The platelet concentration is specifically increased by centrifugation, and/or otherwise fractionation or separation of the red blood cell fraction, e.g. centrifugation of whole blood first by a soft spin such as 8 min at 460 g and the buffy coat is used or further pelleted by a hard spin at higher g values. PRP typically comprises an increased platelet concentration, which is about a 1.5-20 fold increase as compared to venous blood.

Alternatively, “Platelet rich plasma” (PRP) is a blood fraction prepared by separating the red blood cell fraction from a venous blood sample, removing the red blood cell fraction and, if appropriate, the buffy coat, obtaining thereby a platelet poor plasma fraction (PPP), separating—preferably by centrifugation—a platelet rich fraction from the PPP or pelleting platelets, and recovering the platelets in a platelet rich plasma (PRP) fraction, optionally by resuspending the pelleted platelets in an appropriate medium, optionally in PPP.

Such centrifugation and/or fractionation will separate the red blood cells from the other components of blood, and further separate the platelet rich fraction (PRP) including platelets, with or without white blood cells together with a few red blood cells from the platelet poor plasma. PRP may be further concentrated by ultrafiltration, where the protein content of the platelet-rich plasma is concentrated from about 5% to about 20%.

PRP of the prior art typically comprises anticoagulant and clotting is carried out by a clotting agent. However, platelet rich plasma prepared by centrifuging blood without an anticoagulant may be activated by the method as described herein, in particular by clotting, which specifically activates the platelets contained in PRP in the absence of exogenous anticoagulant additives. The present invention specifically provides for activation of PRP, e.g. such that the majority of the platelets are activated. Thus, at least 50% of the platelets in the PRP are activated through the activation of coagulation.

Platelet rich fibrin is clotting spontaneously during its preparation by centrifuging a blood sample, preferably accelerated upon contact with negatively charged surfaces and with adding exogenous coagulation activators.

Preferably, upon clotting and formation of the platelet rich fibrin clot, the acellular or clear supernatant from the PRF may be isolated, or may be removed before fractionating the PRF to isolate the PRF fluid fraction. Such fluid fraction turned out to contain a high concentration of activated platelet releasate and growth factors contained therein.

Preferably, the SPRF may be obtained from a blood sample from a single donor or from multiple donors and mixed together to obtain a single blood sample. According to a specific aspect, the SPRF is obtained from venous blood collected from a single donor. In a preferred embodiment the donor is the patient to whom, once proliferated, the MSCs are reintroduced.

Preferably, the SPRF is employed herein without exogenous anticoagulants that are commonly used in the prior art when preparing PRP, thereby an effective activation of platelets and a content of an activated platelet releasate in the isolated serum fraction is obtained according to the invention.

Preferably, SPRF comprises significantly less bFGF than PRP. Preferably, SPRF comprises less than 100 pg/mL, more preferably less than 50 or 20 pg/mL, highly preferably less than 10 pg/mL, even more preferably less than 5 pg/mL bFGF or essentially comprises no bFGF.

Preferably, SPRF comprises significantly less G-CSF than PRP. Preferably, SPRF comprises less than 100 pg/mL, more preferably less than 50 or 20 pg/mL, highly preferably less than 10 pg/mL G-CSF.

Preferably, SPRF comprises less pro-inflammatory factors than PRP. In particular, SPRF comprises less pro-inflammatory factor(s) than PRP, said pro-inflammatory factor(s) being selected from the group comprising at least IL-6, IL-8, IL-12, TNF-α.

“Culture” as used herein refers to the cultivation of biological material in an artificial environment, i.e. in vitro. Culturing thus may include maintenance and/or propagation of the biological material. The biological material may comprise cells or tissues, including artificial tissues or tissues taken out from an animal body or organs or partial organs or organ parts.

The term “administration” as used herein shall include routes of introducing or applying activated a preparation, such as the serum fraction of the invention, to a subject in need thereof to perform their intended function.

Preferred routes of administration are local, including topical or mucosal application, or application to a wound site or a site of intervention, e.g. surgical intervention, or application to an injured cartilage site or a site of (surgical) intervention at or near to cartilage, or a site in or near to the bone, e.g. under the cartilage. Administration may be carried out e.g. by using a fluid, spray, hydrogel, cream or ointment, or else by any other convenient route, including systemic administration, for example, injections, such as by subcutaneous, or intra-articular injections, by injecting into the layers of skin, under the skin into the epidermis, into fat pads, into muscles of various soft tissues, into cancellous bone and bone marrow, sprayed onto tissue surfaces, mixed with bodily fluids, etc. Various known delivery systems, including syringes, needles, tubing, bags, etc., can be used. Specific or alternative delivery systems employ patches for topical delivery, or implants. Specifically preferred are slow-release preparations, e.g. in the form of a hydrogel, a semisolid or solid gel or formulations and delivery systems to provide for the long-acting treatment. In a preferred embodiment administration is performed in the form of a fluid, in a hydrogel or collagen matrix or an artificial scaffold (matrix).

In one embodiment, the serum fraction of the present invention is the only therapeutically active agent administered to a subject, e.g. as a disease modifying or preventing monotherapy.

The serum fraction can be administered alone, or in combination or conjunction with either another agent or any other therapeutic treatment used in the indication, e.g. used to treat patients suffering from osteoarthritis, osteoarthrosis, bone necrosis, or bone ischemia or a patient in need of cultured cells, e.g. proliferated cells, in particular chondrocytes and/or mesenchymal stem cells.

In another embodiment, the serum fraction of the present invention is combined, e.g. combined in a mixture or kit of parts.

The serum fraction of the present invention may be administered in combination with one or more other therapeutic or prophylactic active agents or regimens, including but not limited to standard treatment, e.g. antibiotics, steroid and non-steroid inhibitors of inflammation, anti-inflammatory agents, vitamins, or minerals.

The serum fraction can be administered prior to the administration of the other agent, simultaneously with the agent, or after the administration of the agent. An alternative delivery system provide for the serum fraction associated with or bound to a carrier material, e.g. a gel or an implant.

The term “in vitro” is understood herein as outside the animal body in an artificial (or laboratory) environment or equipment. Preferably an “in vitro” environment is a controlled environment.

In a preferred embodiment an “in vitro” environment is a cell culture in an artificial vessel.

In a further preferred embodiment “in vitro” environment is an “ex vivo” environment. Ex vivo is understood herein as a body part e.g. tissue or organ or part thereof taken out from the animal body and present in an artificial (or laboratory) environment or equipment. Typically ex vivo refers to experimentation or measurements done in or on tissue from an organism in an external environment. The animal as understood herein is preferably a warm-blooded mammalian, particularly a human being.

The term “isolated” as used herein with respect to a serum fraction shall refer to such fraction of blood, plasma or serum that has been sufficiently separated from other fractions or blood components with which it would naturally be associated. In particular, the serum fraction of the invention is isolated so as to be separated from the PRF coagel and/or from the solid fraction of the PRF coagel. “Isolated” does not necessarily mean the exclusion of artificial or synthetic mixtures with other fractions, compounds or materials, or the presence of impurities that do not interfere with the fundamental activity. In particular, active substances and surgical materials may be combined with the isolated serum fraction of the invention.

The term “pharmaceutically acceptable carrier” as used herein shall specifically refer to any and all suitable solvents, dispersion media, coatings, antibacterial and antifungal agents, isotonic and absorption delaying agents, and the like that are physiologically compatible with a serum fraction provided by the invention. Further examples of pharmaceutically acceptable carriers include sterile water, saline, phosphate buffered saline, dextrose, glycerol, ethanol, and the like, as well as combinations of any thereof. In one such aspect, a serum fraction can be combined with one or more carriers appropriate for a desired route of administration. Such carriers and modes of administration are well known in the pharmaceutical arts. A carrier may include a gel or hydrogel, or gellifying agent or gelling agent, controlled release material or time delay material, or other materials well known in the art.

Additional pharmaceutically acceptable carriers are known in the art and described in, e.g. REMINGTON'S PHARMACEUTICAL SCIENCES. Liquid formulations can be solutions, emulsions or suspensions and can include excipients such as suspending agents, solubilizers, surfactants, preservatives, gelling and chelating agents. Exemplary formulations may be provided, e.g. as a hydrogel including more than 50% water by weight.

The term “subject” or “individual” as used herein shall refer to a warm-blooded mammalian, particularly a human being. In particular, the medical use of the invention or the respective method of treatment applies to a subject in need of prophylaxis or treatment of a disorder or disease condition, e.g. associated with damaged tissue, a wound, an injury, a burn, an incision or an ischemic event, such as osteoarthritis, osteoarthrosis, bone necrosis or bone ischemia, or suffering from such disease condition; in an embodiment the respective method of treatment applies to a subject in need of treatment of a cartilage disorder or disease condition, e.g. associated with damaged cartilage tissue; in a further embodiment the respective method of treatment applies to a subject in need of administration of a pool of MSCs.

The term “patient” includes human and other mammalian, preferably warm-blooded mammalian subjects that receive either prophylactic or therapeutic treatment. The term “treatment” is thus meant to include both prophylactic and therapeutic treatment, in particular to treat, repair or augment a tissue at a target site.

“Stem cells” are undifferentiated or partially differentiated cells with a strong potential to differentiate into several or multiple differentiated cell types and which are also capable of a limited number of cell division to maintain themselves. Thus, stem cells have a limited capability to proliferate and a high potential to differentiate.

“Adult stem cells” (“somatic stem cells” or “tissue stem cells”) are partially differentiated stem cells capable of proliferation, self-renewal, production of a large number of differentiated functional progeny, and are capable of regenerating tissue after injury and having a flexibility in the use of these options.

Without limitation, adult stem cells are e.g.:

Hematopoietic stem cells,

Mammary stem cells,

Intestinal stem cells,

Mesenchymal stem cells,

Endothelial stem cells,

Neural stem cells,

Olfactory adult stem cells,

Neural crest stem cells,

Testicular cells.

“Mesenchymal stem cells” (MSCs) are stem cells of stromal origin and/or localization which have the potential to differentiate into several cell types, and are
adherent,
capable of differentiation into mesenchymal tissue, preferably bone, cartilage or adipose tissue in vitro, and preferably are
CD105, CD73 and CD90 positive, do not carry surface markers of blood progenitor cells or heamatopoietic stem cells, and preferably are CD45, CD34, CD14, CD11b, CD79a and CD19 negative.

“Cell therapy” is the transplantation of human or animal cells to a patient to replace or repair damaged tissue.

“MSC therapy” is a cell therapy wherein MSCs are administered to a patient having an impaired tissue and wherein said MSCs are differentiated into cells of said tissue or tissue-specific cells or tissue-resident cells in the patient.

“Osteoarthritis” is a degenerative disease characterized by erosion of articular cartilage, which becomes soft, frayed, and thinned with eburnation of subchondral bone and outgrowths of marginal osteophytes; results in pain and loss of function; mainly affects weight-bearing joints. Osteoarthritis is also called degenerative joint disease, or osteoarthrosis. Osteoarthrosis may be considered as a chronic noninflammatory bone disease variant and also may be a synonym for osteoarthritis.

“Spongy bone” is the tissue that makes up the interior of bones; “compact bone” is the tissue that forms the surface of bones. In long bones, spongy bone forms the interior of the epiphyses.

“Osteonecrosis” is bone death in particular caused by poor blood supply.

“Chondrocyte dedifferentiation” is a process which involves the switching of the cell phenotype towards a state where extracellular matrix production no longer occurs. “Chondrocyte dedifferentiation” is also understood herein as a phenomenon that occurs during chondrocyte expansion in culture on 2D substrates.

“Chondrocyte redifferentiation” is a process which involves the switching of the cell phenotype from a dedifferentiated state (e.g. a state obtained by chondrocyte expansion in culture on 2D substrates) into a more differentiated state.

The dedifferentiation or the redifferentiation process can be monitored by differentiation markers e.g. by the Col II/Col I ratio.

“Dedifferentiated chondrocytes” as used herein are chondrocytes wherein the Col II/Col I ratio (CONSTANS-LIKE 1 and 2 are zinc finger proteins) is significantly lower than in healthy control chondrocytes.

In particular, dedifferentiated chondrocytes show reduced extracellular matrix production in comparison with healthy chondrocytes; in particular, dedifferentiated chondrocytes show reduced expression of aggrecan and collagen type II, in particular collagent type IIB in comparison with healthy chondrocytes.

Preferably dedifferentiated chondrocytes are chondrocytes which have been subjected to 2-dimensional culturing and/or cell expansion.

In an embodiment dedifferentiated chondrocytes are arthritic chondrocytes or chondrocytes dedifferentiated due to a cartilage disease or in impaired cartilage.

“Chondrocyte proliferation” (or “chondrocyte expansion”) is a process which, upon culturing (and expansion) of chondrocytes, involves the propagation or multiplication of the cultured chondrocyte cells.

The term “comprise(s)” or “comprising” or “including” are to be construed herein as having a non-exhaustive meaning and to allow the addition or involvement of further features or method steps or components to anything which comprises the listed features or method steps or components. Such terms can be limited to “consisting essentially of” or “comprising substantially” which is to be understood as consisting of mandatory features or method steps or components listed in a list, e.g. in a claim, whereas allowing to contain additionally other features or method steps or components which do not materially affect the essential characteristics of the use, method, composition or other subject matter.

As used in this specification and the appended claims, the singular forms “a”, “an” and “the” include plural references, and should be construed as including the meaning “one or more”, unless the content clearly dictates otherwise. In general, it is also to be understood that the terminology used herein is for the purpose of describing particular embodiments only, and is not intended to be limiting.

### DETAILED DESCRIPTION OF THE INVENTION

The biological properties of the serum fraction or the respective pharmaceutical preparations of the invention may be characterized in vitro, preferably ex vivo in cell or tissue experiments or in whole organism experiments. As is known in the art, drugs are often tested in vivo in animals, including but not limited to mice, rats, rabbits, dogs, cats, pigs, and monkeys, in order to measure a drug's efficacy for treatment against a disease or disease model, or to measure a drug's pharmacokinetics, pharmacodynamics, toxicity, and other properties. The animals may be referred to as disease models. The serum fraction and respective pharmaceutical compositions of the present invention may further be tested in humans to determine their therapeutic or prophylactic efficacy, toxicity, immunogenicity, pharmacokinetics, and/or other clinical properties.

The terminology of these platelet concentrates, including PRP, PRF, platelet gel, fibrin glue and also platelet poor plasma (PPP) remains uncertain and their effect—despite the several positive results obtained in certain situations, controversial. A general classification of these products is suggested by Dohan et al. (“In search of a consensus terminology in the field of platelet concentrates for surgical use: platelet-rich plasma (PRP), platelet-rich fibrin (PRF), fibrin gel polymerization and leukocytes.” Curr Pharm Biotechnol. 2012 June; 13(7):1131-7.). Bone ischemia or ischemic bone necrosis (avascular necrosis, osteonecrosis, bone infarction, aseptic necrosis) is a disease wherein cellular death (necrosis) of bone components is due to an interruption of the blood supply of the bone tissue. As a result, the bone tissue dies; this necrosis of cell touches at the first place hematopoietic cells. If the disease affects the bones of a joint, it probably leads to destruction of the joint articular surfaces. Ischemic bone necrosis may be caused e.g. by traumatic injury, fracture or dislocation of the bones, dislocated hip or excessive alcohol consumption or use of steroids.

Upon reperfusion, repair of ischemic bone occurs. At first, mesenchymal cells and macrophages migrate from the living bone tissue grow into the dead bone marrow spaces and then the mesenchymal cells differentiate into osteoblasts and fibroblasts.

Possible treatment includes the replacement of the dead tissue and/or the use of compounds, which may reduce the rate of bone breakdown. There is still a need, however, for materials, which facilitate bone regeneration after the ischemic event.

Recent advances in regenerative medicine shed light on the capabilities of various growth factors, which have remarkable effects as inducers of bone formation. In addition to bone morphogenic proteins, platelet-derived growth factor (PDGF), transforming growth factor beta (TGF-beta), insulin-like growth factor (IGF) and epidermal growth factor (EGF) also have a positive effect on bone regeneration. Single factor therapies are available as recombinant products, currently BMP-2, -7, and PDGF have marketing approval, or as natural extracts typically isolated from venous blood.

Activation

In the prior art, the PRP was typically produced as anticoagulated preparation, e.g. from blood or PRP collected with anticoagulants, such as heparin, citrate, acid citrate dextrose (ACD) and/or citrate-theophylline-adenosine-dipyridamole (CTAD). Such anticoagulants were known to preserve the platelets maintaining the integrity of platelet structures. In contrast, the present invention is based on the PRP plasma activation wherein said PRP does not contain such anticoagulants, which was found to be supporting the effective production of invaluable growth factors and cytokines which are released by the platelets activated according to the method of the present invention.

The serum fraction of the invention is prepared without exogenous anticoagulants that are commonly used when preparing PRP, thereby an effective activation of platelets and a content of an activated platelet releasate in the isolated serum fraction is obtained according to the invention.

Specifically, the PRP is clotting spontaneously during its preparation by centrifuging a blood sample, preferably accelerated upon contact with negatively charged surfaces and with adding exogenous coagulation activators.

According to a specific aspect, the blood sample is collected in a clot device such as a clot tube or clot syringe, optionally wherein the PRP is prepared and clotted to obtain the coagel, e.g. a clot activating tube or syringe, which is typically equipped with appropriate coagulation initiators or accelerators, herein referred to as “coagulation activators” or contact activators.

Particularly preferred contact activators are anorganic, physical and/or biologic contact activators. According to a specific aspect, coagulation is accelerated or activated through the contact activation pathway, specifically upon contact with negatively charged surfaces, preferably glass e.g. silicate, borosilicate; kalomel, diatomaceous earth polymers with a polar structure, e.g. acrylates, carbonates, or polyacrylamides, specifically those which are physical contact activators.

Alternative activators may be biological, or chemicals like collagen, CaCl₂, Ca-gluconate, MgCl₂, thromboxane A2, ADP, thrombin, D-glucose, dextran, glycerol. These activators may be present as a coating, bead, or porous sponge. The activator may also be an enzyme or amino acid, like thrombin, thromboplastin or coagulation factors e.g. FIIa, FXa, FVIIa, FIXa, FXIa, FXIIa, FXIVa.

According to specific embodiments, it is preferred to prepare the serum fraction by endogenous clotting of the PRP, i.e. by physical contact with suitable surfaces only, thereby avoiding exogenous additives which would possibly contaminate the serum preparation. Such endogenous clotting would provide for the endogenously activated platelets, allowing the collection and isolation of the fluid fraction of PRF or the serum fraction of the invention containing the activated platelet releasate obtained from such activated platelets without exogenous contaminants. Specifically, in such embodiment the addition of exogenous thrombin or other coagulation factors is avoided.

For example, typical clot tubes may provide a negatively charged contact surface, such as glass, which would accelerate spontaneous clotting of the PRP during separation of the red blood cell fraction. The device may not only be used for collecting the blood, but also for preparing the PRP, e.g. by centrifugation of the blood sample in one or more consecutive steps.

According to a specific aspect, the serum fraction is freshly prepared without adding preservatives, such as ethanol, and e.g. prepared without any intermediate storage or freezing/thawing step. Typically, the preparation method would be carried out during a short period of time to obtain a freshly prepared serum fraction, e.g. a period up to 10 hours, preferably less than 6 hours.

Preservation

Such serum fraction is e.g. prepared ready-to-use for the purpose of treating a patient without using a preservative. Thus, stabilizing agents, such as high concentrations of alcohol or further preservatives are avoided. Yet, the freshly prepared serum fraction is storage stable at lower temperatures and may be stored at refrigerating temperatures or frozen over a longer period of time, e.g. at 2° C.-12° C. for up to 1-24 months, or at −80° C. to −25° C. for up to 0-5 years.

In the present invention SPRF showed surprisingly consistently better results than PRP and similar to or even better than the gold standard cell medium supplement FBS plus growth factors. However, FBS obviously is not appropriate for medicine and is not advisable in cell cultures in transplantation applications.

The invention relates to in vitro, ex vivo or in vivo methods of treatment of cells which comprise a cell culture and an incubation step, e.g. in solution or on a solid support, e.g. an implant or bone graft material. Such cell culture or treatment is preferably performed in the following way: Cells are cultured under regular cell culture conditions and the serum fraction of the invention is added to the medium. The addition of the serum fraction specifically induces cell proliferation, prevents cell death or damage and may induce differentiation in specific cell types. Cell proliferation is typically measured by cell counting or surrogate methods.

It was also unexpectedly found that certain blood derived preparations accelerate and improve cell proliferation, regeneration and healing of tissue, in particular osteoarthritic material or the bone tissue after ischemic bone damage.

Blood cells, upon activation by injury, secrete a plethora of proliferation factors into the serum. This raises the possibility of using serum products for therapeutic targets other than acute injury, thus applying a more physiological growth factor mix than the monotherapy of recombinant proteins. Investigations of PRP and related serum fractions in an ex-vivo model of bone ischemia were made. Small bone pieces of 10 mm³ were isolated from the discarded femoral heads during hip replacement operations. The explants were grown in culture for 3 days then subjected to transient oxygen glucose deprivation (OGD) for simulating ischemia. The majority of the cells on the bone explants died and the survivors did not proliferate. Adding PRP that is either native or anticoagulated (heparinized) or activated by chemical or physical means, did not have any effect on the postischemic cells. However, the serum fraction of the invention, in particular containing the fluid fraction of the coagel of PRF, in particular the serum pressed from platelet rich fibrin (SPRF), induced cell proliferation of the post-ischemic osteoblasts. Proteome-profiler analysis showed that PRP and SPRF have diverging growth factor profiles, with platelet factor 4 being a key one which has a higher concentration in SPRF than PRP. Another significant difference is the lack of fibrin or fibrinogen in SPRF. It is concluded that the serum fraction of the invention, in particular the SPRF, is a blood derivative which can restore the cell proliferation capacities, e.g. of post-ischemic bone and thus can be a new therapeutic tool, with a specific use in degenerative bone diseases.

The serum fraction of the invention is specifically provided for treating osteoarthritis, osteoarthrosis, bone necrosis or bone ischemia, for implants or autologous bone grafts to prevent or treat ischemia after implantation, or to increase proliferation of cells after an ischemic episode.

Bone ischemia or avascular necrosis (AVN) for example of the femoral head still presents a challenge for the orthopedic surgeons, mainly for the progressive characters of the disease and the relative young age of the patients. Presently available specific and efficient treatments are:
core decompression
autologous bone
demineralized bone-matrix
BMP (Bone morphogenic proteins)
osteotomia
application of promising agents of human blood, e.g. PRP
any combination of the foregoing.

A human in vitro model was set-up and the effects of blood plasma derived preparations in the pathomechanism of bone ischemia were tested.

Experiments with various plasma fractions were carried out and it was surprisingly found that preparations can be obtained which are effective for accelerating and facilitating bone regeneration after bone ischemia.

The ex vivo results showed that the serum derived preparation of the invention directly induces proliferation of bone cells even after severe ischemia. Proliferation of cells has been found to be significantly improved by the fluid fraction of PRF, which comprises or consists of the liquid content in PRF, but not by PRP of the prior art.

The experimental results were surprising in view of the prior art. It was specifically surprising that the starting material, which is PRP without the addition of anticoagulants, and the clotting according to the invention affects the final result. Specifically, the freshly prepared serum fraction of the invention could be provided as an improved material for medical use.

Activated fibrin has a strong pro-inflammatory effect which is beneficial in case of acute injuries but may be harmful in chronic cases where regeneration of the tissues is inhibited by persistent inflammation. Therefore, matching the right kind of proliferation factor mix with a certain pathology is necessary in order to develop a reliable clinical protocol. In the present study a novel ex vivo human model of bone ischemia was used, which closely resembles the tissue states of transplanted bone or tissue damaged by end-stage degenerative diseases. The constituents of various platelet-rich serum fractions were analyzed and their effects as proliferation factors on postischemic human bone explants were investigated, to confirm the positive effects of the serum fraction of the invention.

Without being bound by theory this is possibly the mechanism behind the clinical observation that PRP augmented bone grafts have a markedly better 6-year result than decompression therapy in femoral head necrosis.

Specific method steps applicable in the present invention are as follows:

1. Obtain venous blood. No additives, e.g. anticoagulants, are necessary.

2. Remove red blood cells.

3. Obtain platelet rich fibrin (a yellowish coagulum floats on top of the red blood cell fraction).

4. From PRF separate the fluid fraction and the matrix (solid fraction). This can be done by pressing (squeezing) the PRF or by centrifugation at an increased, appropriate force.

In a preferred embodiment spinning down is carried out within 20 minutes, preferably within 15, 10, 5 minutes, or shorter period from obtaining venous blood.

Preferably, centrifugation is carried out at 1000 g to 5000 g, preferably at 2000 g to 4000 g or 1000 g to 3000 g or 1000 g to 4000 g, more preferably at about 1200 g to 2500 g or at about 1500 g to 2000 g. Preferably, centrifugation is carried out for 2 to 20 minutes, preferably for 4 to 15 minutes, highly preferably to about 5 to 12 minutes, preferably about 10 minutes (+/−2 minutes).

The clot obtained (i.e. the coagel) can be removed by any appropriate method, e.g. by filtering or other physical means. In a preferred embodiment continuous centrifugation is applied and the clot is removed at an opening on the wall of the centrifugation space.

The fluid fraction from the clot can be removed by squeezing, pressing, filtering, vacuum filtering or any other appropriate method.

The process can be carried out in an application device e.g. a syringe. Preferably, according to a specific aspect, the serum fraction is freshly prepared and ready-to-use, optionally wherein the serum fraction is provided in the application device. A particular embodiment refers to an autologous serum fraction, i.e. a serum fraction prepared from blood of a single individual donor which is for administration to the same individual. Alternatively, a pooled serum fraction is prepared from multiple patients. In a preferred embodiment the SPRF is prepared from donors of young age, e.g. by donors from 19 to 40 years old e.g. by donors from 20 to 35 years old.

In a preferred embodiment the donor subject is different from the patient. Preferably, the age of the donor subject is below 50 years, preferably below 40 years, more preferably below 35 or 30 years. In a preferred embodiment the age of the patient is above 50 years or above 55 years or above 60 years.

The serum fraction may be conveniently prepared in an appropriate preparation device suitable for aseptic collection of the blood. Negatively charged surfaces are preferred. The isolated serum fraction may be produced in the application device in an aseptic way and may conveniently be directly and immediately administered to the individual, e.g. by an applicator aseptically connected to the preparation device, or by a separate application device or kit which allows the aseptic transfer of the prepared serum fraction to the application device and/or to administer the preparation to the individual.

According to the invention, the serum fraction is specifically provided for use in the manufacturing of an autologous pharmaceutical or medicinal product. Such product may be in the form of a pharmaceutical preparation or a medical device preparation.

Specifically, the serum fraction is provided for the treatment of the serum fraction's donor. Specifically, the autologous use of the serum fraction is preferred.

The invention is particularly useful in helping, facilitating or allowing the regeneration of the bone tissue of a subject. Bone tissue can be acutely damaged such as in case of trauma or surgery or can be chronically impaired e.g. in case of degenerative bone diseases such as osteoarthrosis, bone necrosis, or bone ischemia. As an example, ischemia can be present during transplantation of bone tissue or organs containing bone such as osteochondral plugs. Specific methods, which can be improved by using the serum fraction of the invention, are e.g. methods to apply plasma preparations in surgery such as taught in the following publications.
Jun Araki et al.: Optimized Preparation Method of Platelet-Concentrated Plasma and Noncoagulating Platelet-Derived Factor Concentrates: Maximization of Platelet Concentration and Removal of Fibrinogen (Tissue Eng Part C Methods. 2012 March; 18(3):176-85).
Dohan, D. M. et al.: Platelet-rich fibrin (PRF): A second-generation platelet concentrate. Part I: Technological concepts and evolution. Oral Surg Oral Med Oral Pathol Oral Radiol Endod. 2006; 101(3):E37-44.

Mesenchymal Stem Cells (MSCs)

In a particular aspect, the cells cultured in the presence of SPRF are mesenchymal stem cells.

One of the most important functions of MSCs is natural tissue repair, which is mainly the result of the wide distribution and multipotent differentiation in the human body. Clinical and preclinical models already proved this reparative effect and the critical role of MSCs in injury healing was strongly suggested as well. MSCs are believed to be responsible for replacing cells that are lost in diseases or pathological conditions. Due to these functions the approach of supplementing stem cells to enhance tissue regeneration and treat degenerative diseases were also successfully tested and were shown to be effective. MSCs are also responsible for therapeutic effects in the musculoskeletal system, and were found to be effective in periodontal tissue and bone damage caused by e.g. osteonecrosis and has been successfully applied in cartilage and long bone repair. Besides supplementing MSCs, which were harvested from the patient and either injected or cultured and injected back to the patient, there may be an alternative solution as well. The distribution of stem cells may alternatively be redistributed using our method, which basically enables selective proliferation of the available stem cells, which means that the proliferative effect can be localized and thus a selective tissue repairing treatment can be realized. In order to overcome the uncertainty, which is posed by the circulating excess stem cell concentration, we focus on local therapeutic effect, which means that musculoskeletal and degenerative bone and joint diseases are the main therapeutical targets. This solves the majority of the circulation problems as the circulation of these parts of the body is limited thus the effect of the enhanced proliferation of the stem cells is concentrated on local tissue repair. In our case these tissues are mainly musculoskeletal tissues, more specifically bone and joint tissue.

Another important aspect of our application is that the MSCs preserve their stem cell character during the first 5 days of culturing as no differentiation occurs into adipocyte direction with the use of our SPRF culture supplement except the increase in osteoblast factors after 5 days of culture or further culturing. This enables the advantage of not interfering with the MSCs, thus the MSCs will only differentiate as an effect of the surrounding cells at site of the treatment. This gives the opportunity that the stem cells will differentiate in a manner that accelerates the regeneration of the treated tissue.

In case of diseases of the bone and cartilage this osteogenic differentiation indicates a further unforeseen advantage as it shows that SPRF in a surprising manner prepares the cells for osteogenic differentiation whereas no other direction of differentiation is observed. This means that SPRF and MSCs proliferated thereby are particularly suitable for treatment of diseases of the bone, in particular osteoarthrosis and osteoarthritis.

Source of MSCs

While MSCs are available from various sources it appears the present invention is not limited to BM derived MSCs and also for example adipose derived MSCs could be applied. Literature opinions vary in assessing the capabilities of MSCs of various sources, an advantage of the present invention may be that due to culturing as disclosed herein multiple sources may become useful and available.

Culturing MSCs

In the present invention MSCs are obtained from a subject and said MSCs are cultured by an in vitro method, as disclosed in the Brief Description of the Invention.

In the invention usual MSC culturing conditions can be applied, for example a DMEM basal medium with sugar source like high glucose, glutamate source like GlutaMax™ Supplement, pyruvate and antibiotics or selective agents like penicillin/streptomycin and 1% amphotericin. Instead of FBS regularly used in media like DMEM, SPRF and only SPRF are to be used. In a particular embodiment even no further growth factors are to be used.

SPRF as an MSC Medium Supplement/Additive

Pressing out the fluid content from PRF leads to an autologous blood separation product, which does not contain fibrinogen, anticoagulants and the inflammation markers are low. After testing it as a stem cell medium supplement, the inventors have surprisingly found that use of a serum from platelet rich fibrin (SPRF) instead of PRP and FBS enhanced the proliferation rate of human mesenchymal stem cells in vitro while phenotypical changes were not observed and differentiation potential of proliferated MSCs was maintained. Moreover, culturing human subchondral bone pieces in SPRF supplemented medium cell viability was not only retained, but also significantly increased in 7-days culture without any measurable cell differentiation. The inventors revealed that predominantly mesenchymal stem cells were multiplicated in the course of the incubation time.

Treatment with MSCs

Osteoarthritis (OA) is one of the most prevalent joint diseases with prominent symptoms affecting the daily life of millions of middle aged and elderly people. Despite this, there are no successful medical interventions that can prevent the progressive destruction of OA joints.

Administration of SPRF in Osteoarthritis

Administration of SPRF is conveniently carried out by injection at the site of impaired bone or chondrocyte tissue. In order to maintain an appropriate level so as to maintain contact with MSCs multiple injections can be applied. For example, injection can be added regularly, e.g. every day or in every 2 days or 1, 2 or 3 times a week.

Another possibility to maintain the level of SPRF may be e.g. matrix assisted administration.

Effect of SPRF on Chondrocytes

Chondrocytes are the main cell type found within cartilage. They are responsible for the synthesis and maintenance of the extracellular matrix (ECM) and are themselves isolated from each other by a large quantity of ECM. Chondrocytes therefore must exist in a low oxygen environment; all this explains the unitary nature of chondrocytes, cartilage and its low reparative potential and thus the problems arising in clinical conditions like osteoarthritis (OA).

Although articular cartilage can well tolerate physical stress, its ability to heal even a minor injury is particularly low which makes the cartilage tissue particularly sensitive to degenerative processes. Ageing also leads to alterations in ECM composition and alters the activity of the chondrocytes, including their ability to respond to stimuli such as growth factors. Moreover, chondrocytes gradually decline in number with age.

Other typical conditions wherein damage or injury of cartilage occurs are e.g. the following:

Cartilage damage like mechanical cartilage injury, traumatic cartilage loss, cellular matrix linkage rupture, chondrocyte protein synthesis inhibition, chondrocyte apoptosis, cartilage ulcer, subchondral bone damage, Ahlback's disease, osteochondral lesions.

In any of these situations the patient may be a subject in need of cartilage repair, preferably articular cartilage repair, e.g. cartilage replacement therapy.

Autologous chondrocyte implantation (ACI) is one of the most widely used cell based repair strategies for articular cartilage [Brittberg, M. et al. Treatment of deep cartilage defects in the knee with autologous chondrocyte transplantation. N Engl J Med. 1994, 331(4): 889-95; Brittberg M. Autologous chondrocyte implantation—technique and long-term follow-up. Injury. 2008, 39(Suppl. 1):S40-9.]

In this method cartilage biopsy is taken from the patient from an area which is not weight bearing and preferably which is intact. Then chondrocytes are isolated and transferred into a 2 dimensional culture wherein they are cultured (expanded) to increase their number (multiply them). The so cultured chondrocytes then introduced into the affected (damaged, impaired) area of the cartilage wherein they are fixed.

Chondrocyte Dedifferentiation

The zones of cartilage are based on the shape of the chondrocytes, the composition of the extracellular matrix (ECM) and the orientation of the type II collagen with respect to the articulating surface and the subchondral bone. [Mobasheri, A. et al. Chondrocyte and mesenchymal stem cell-based therapies for cartilage repair in osteoarthritis and related orthopaedic conditions. Maturitas, 2014, 78: 188-198.]. Damaged ECM or its complete absence will result in a major shift in chondrocyte gene expression. Instead of producing cartilage specific proteoglycans and collagen type II, chondrocytes switch to making non-specific proteoglycans and collagen type I.

Chondrocyte dedifferentiation is known to influence cell mechanics leading to alterations in cell function. Dedifferentiation occurs in diseased, e.g. OA cells as well. Typically metabolically active OA chondrocytes no longer express aggrecan and collagen type II. However, chondrocytes in OA cartilage express collagens type I and III, which are rare in normal articular cartilage. OA chondrocytes also express type IIA collagen, a marker of prechondrocyte phenotype. This expression is enhanced by transforming growth factor-1 (TGF-1). Bone morphogenetic protein-2 (BMP-2), on the other hand, favors the expression of type IIB collagen isoform, a normal component of articular cartilage. Thus, despite their high synthetic activity, dedifferentiated chondrocytes do not express cartilage-specific anabolic genes such as aggrecan or type II collagen, associated with an impairment in anabolic function.

Thus, damaged or ostearthritic chondrocytes show signs of dedifferentiation which is modeled in 2 dimensional chondrocyte cultures used for autologous cell based repair of articular cartilage. While the US Food and Drug Administration has approved the clinical use of chondrocytes that have been expanded in vitro to obtain larger number of cells, cells cultured using traditional 2D monolayer conditions undergo dedifferentiation indicated by a phenotypic shift. Dedifferentiated cells are larger, spread and acquire actin stress fibers. They express fibroblastic matrix (such as type I collagen; COL1A1) as well as contractile (alpha smooth muscle actin; aSMA) molecules resulting in biomechanically inferior tissue capable of shrinkage. [Parreno, J. et al., Chondrocyte Dedifferentiation: Actin Regulates the Passaged Cell Phenotype Through MRTFa. Osteoarthritis and Cartilage, 22 (2014), S57-S489, abstract].

The term dedifferentiation as used herein includes both dedifferentiation due to a disease process and the process as occurs in vitro, preferably the latter.

In this study, we have investigated the effect of serum from platelet rich fibrin (SPRF) donor age variation on osteoarthritic (OA) chondrocyte culture expansion. SPRF was prepared from 10 individual donors aged 25 to 59 years and added to chondrocyte cultures started from explants harvested at knee replacement surgery.

Multiplex Array

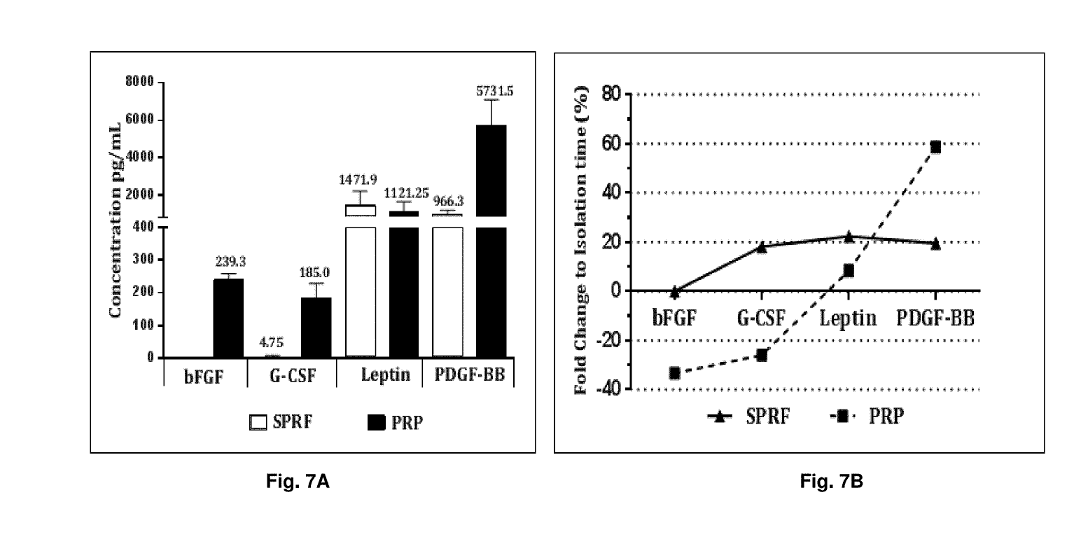

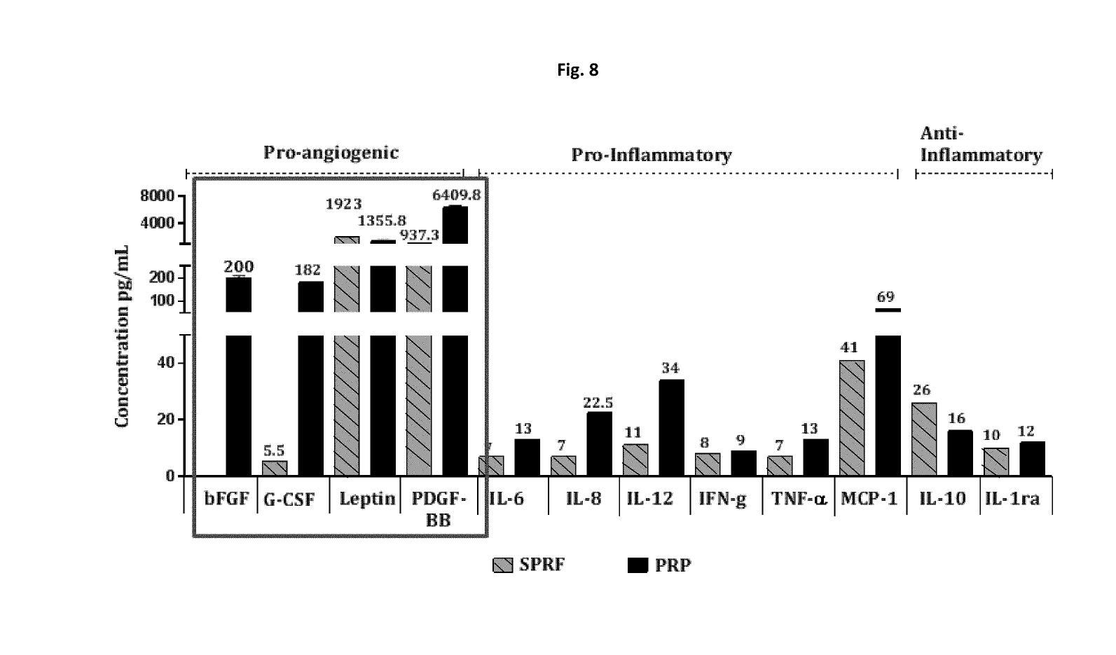

We have performed a multiplex array—protein quantification (FIGS. 7A, 7B and 8) to characterize the fractions.

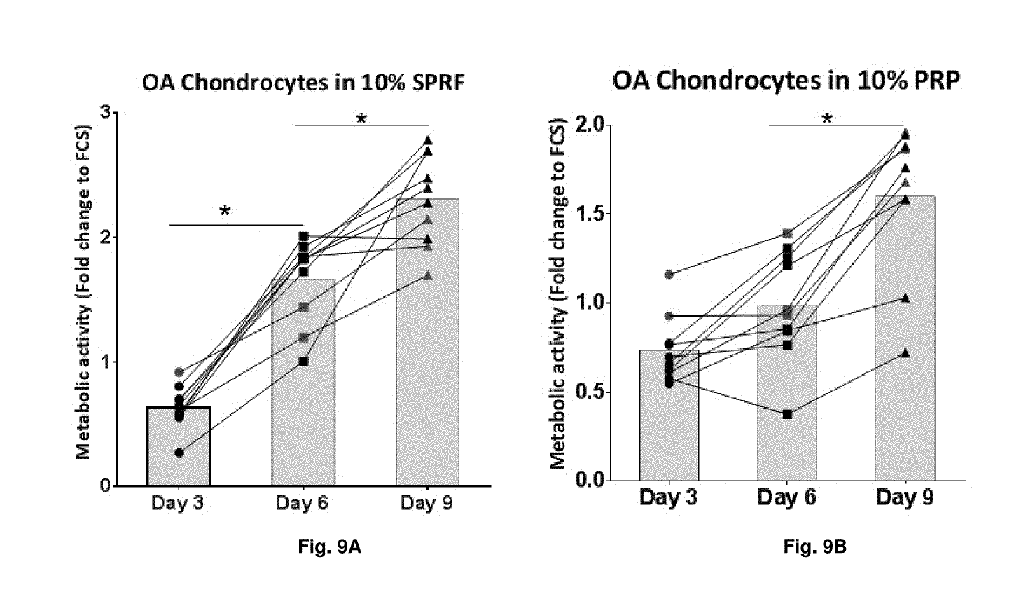

In cell proliferation experiments of chondrocytes from OA patients we have surprisingly seen that SPRF was slightly more effective than PRP in particular from younger donors in promoting proliferation (see e.g. FIGS. 9A and 9B).

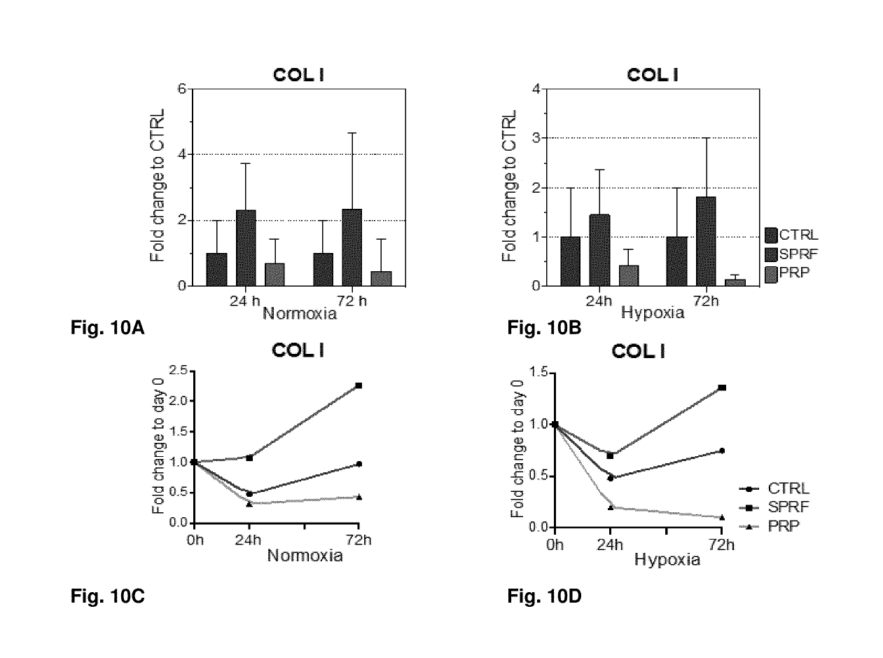

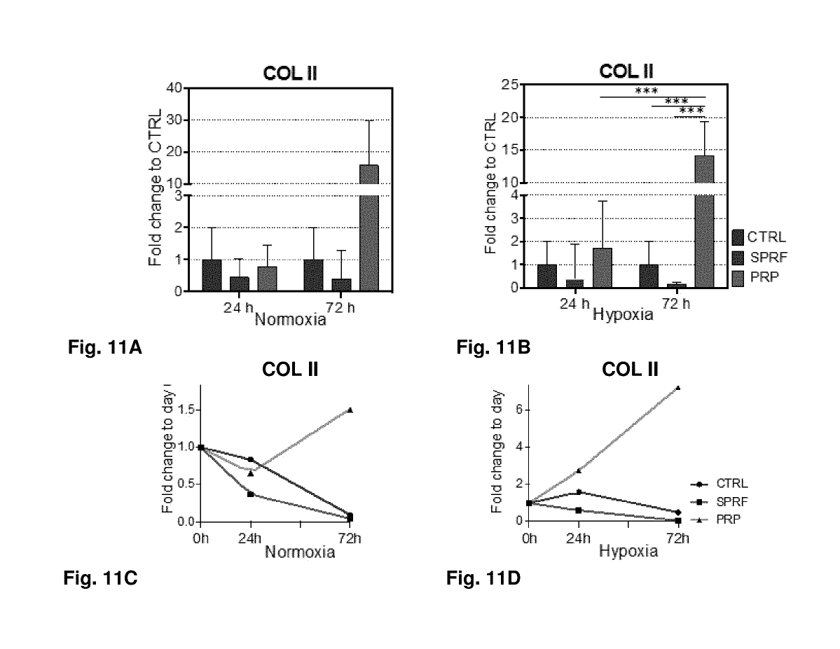

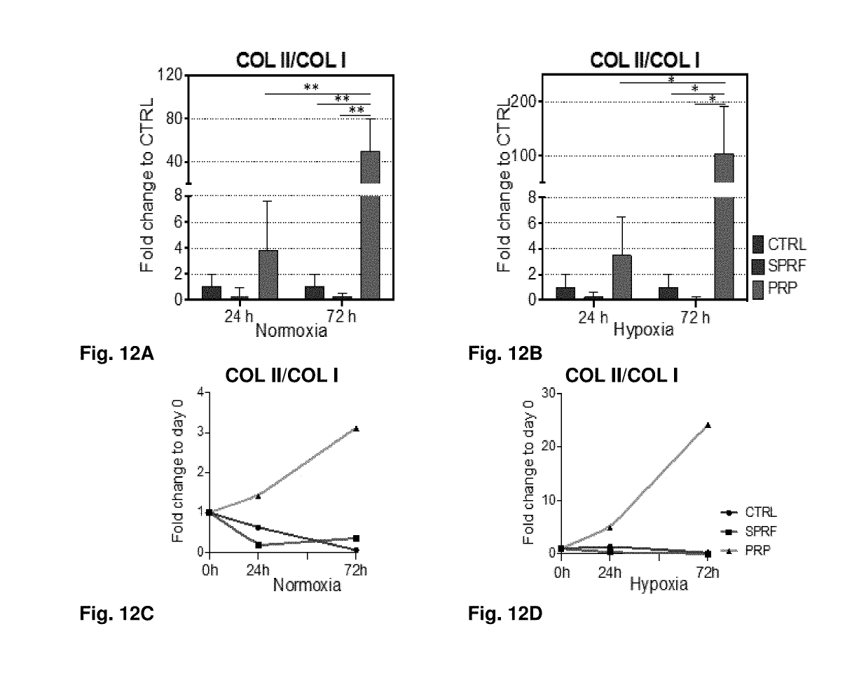

Col I and COL II (CONSTANS-LIKE 1 and 2) are zinc finger proteins playing role in gene regulation. The Col II/Col I ratio is indicative of the cell-cell differentiation (in dedifferentiated cell the redifferentiation) potential of cells. Measurement may be made at the mRNA or protein level. We have shown that this index of differentiation is very low when SPRF is used both under conditions of normoxia and hypoxia (FIGS. 10A-D, 11A-D and 12A-D).

Proteins of the matrix metalloproteinase (MMP) family are involved in the breakdown of extracellular matrix proteins and during tissue remodeling in normal physiological processes, such as embryonic development and reproduction. MMP 3 is involved in normal collagen breakdown. MMP13 is expressed in the skeleton as required for restructuring the collagen matrix for bone mineralization. In pathological situations it is highly overexpressed; this occurs in human carcinomas, rheumatoid arthritis and osteoarthritis [Johansson, N., Ahonen, M., Kahari, V.M. (2000). “Matrix metalloproteinases in tumor invasion.” Cell Mol Life Sci. 57 (1): 5-15].

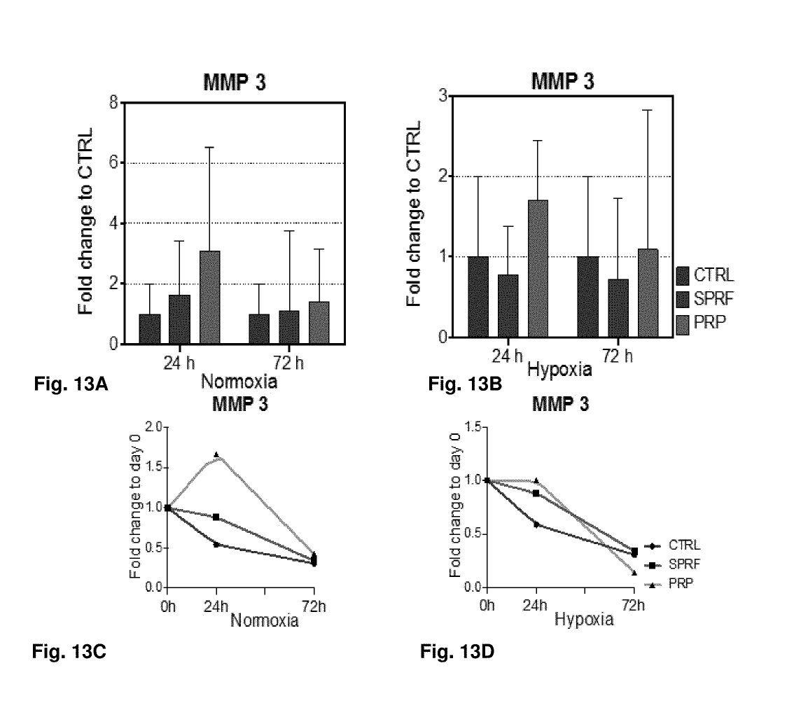

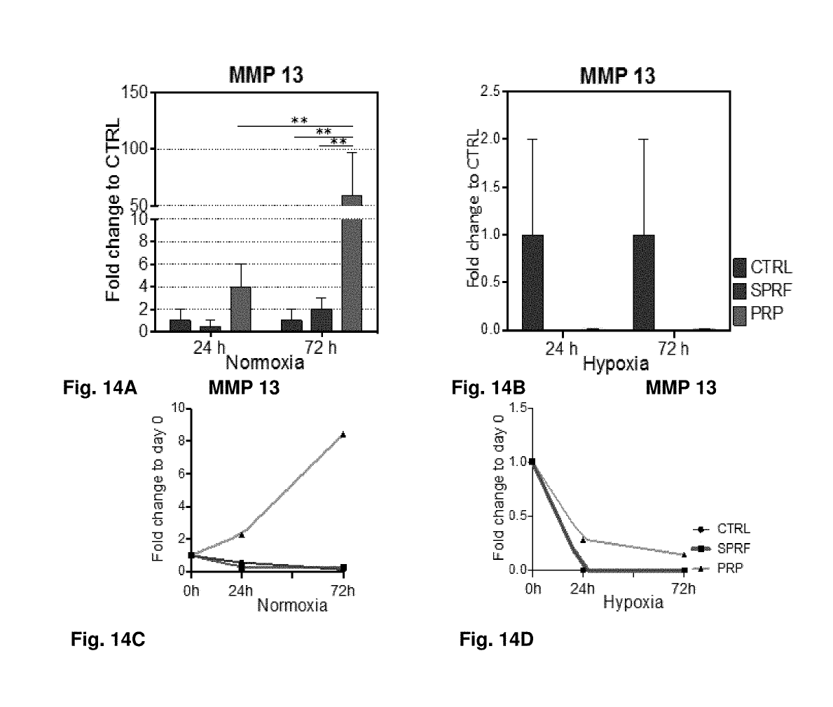

FIGS. 13A-D and 14A-D show a normal expression of these genes under the condition of normoxia. Matrix metalloproteinase 3 (MMP 3) gene expression was normally regulated also under conditions of hypoxia. Matrix metalloproteinase 13 (MMP 13) gene expression was reduced under conditions of hypoxia in case of SPRF and PRP which is normal and suggest that SPRF is safe.

Chondrocyte Transplantation or Implantation

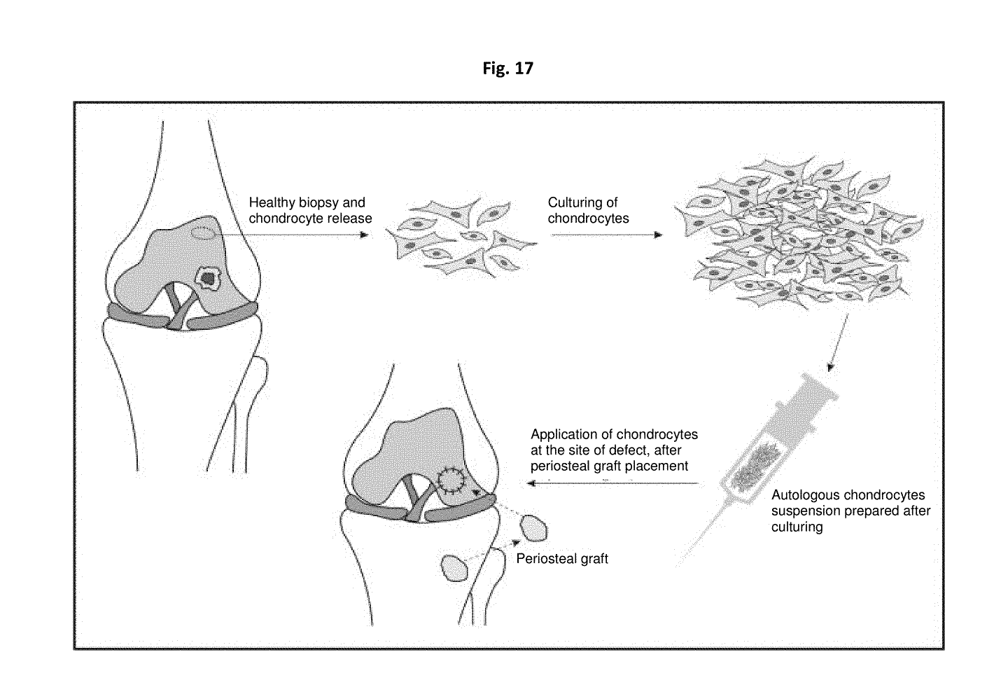

FIG. 17 shows an autologous chondrocyte implantation process scheme. Such methods are known e.g. in the literature below, the content of which is incorporated herein by reference, as far as treatment of cartilage with chondrocytes is disclosed. Autologous chondrocyte implantation is described e.g. in Robi et al, Current Issues in Sports and Exercise Medicine (2013)/978-953-51-1031-6.]. Also, a review is provided by Vasiliadis, H. et al. [Vasiliadis, H.; Wasiak, J.; Salanti, G. (2010). “Autologous chondrocyte implantation for the treatment of cartilage lesions of the knee: a systematic review of randomized studies”. Knee Surgery, Sports Traumatology, Arthroscopy 18 (12): 1645-1655.]. A further summary is provided by Mobasheri, Ali et al. [Mobasheri, Ali et al. Chondrocyte and mesenchymal stem cell-based therapies for cartilage repair in osteoarthritis and related orthopaedic conditions. Maturitas 78 (2014) 188-198]. Peterson, Lars et al. [Peterson, Lars et al. Autologous Chondrocyte Implantation: A Long-term Follow-up. The American Journal of Sports Medicine, 2010, 38(6) 1117-1124.] report on a long term follow of patients treated by first generation ACI and conclude that ACI is an effective and durable solution for the treatment of large full-thickness cartilage and osteochondral lesions of the knee joint. They add that second- and third-generation ACI techniques have been developed since 1987, using either manufactured coverings or scaffolds for the 3-dimensional culturing of the chondrocytes or, in third generation

ACI, fully arthroscopical administration can further improve outcomes in the future.

The present inventive methods provided herein seamlessly fit into such scheme by adding SPRF to the chondrocyte culturing medium. SPRF is added to the culturing medium of chondrocytes so as to improve their proliferation rate, preferably in vitro. SPRF is in one embodiment simply mixed into the media. Upon implantation of the cells SPRF may be administered simultaneously.

Autologous Chondrocyte Implantation with Scaffold (Matrix) or Cover

Variants of the autologous chondrocyte implantation method are methods using a cover for the chondrocyte, e.g. a periosteum-cover technique e.g. wherein type I/type III collagen is used as a cover (ACI-C) and matrix-induced autologous chondrocyte implantation (MACI) using a collagen bilayer seeded with chondrocytes. The methods are described e.g. in Bartlett W. et al. [Bartlett W. et al. Autologous chondrocyte implantation versus matrix-induced autologous chondrocyte implantation for osteochondral defects of the knee. J Bone Joint Surg [Br] 2005; 87-B:640-5.]. A more recent report on MACI is provided by Schneider, T., E. and Karaikudi S. [Schneider, T., E., Matrix-Induced Autologous Chondrocyte Implantation (MACI) Grafting for Osteochondral Lesions of the Talus. Foot & Ankle International 30(9) 2009]. The studies report that the MACI technique is a basically reliable treatment method. According to the invention such method can be used with a minimal adaptation wherein the chondrocytes are to be cultured in SPRF.

Upon culturing the chondrocytes the SPRF is preferably used in a concentration of between 1-25% or 2-20%, preferably 5 to 15%, highly preferably 8 to 12% or in particular about 10%, wherein the percentage of concentration is given in v/v %.

Administration SPRF to the Site of Cartilage Damage

An alternative method for the treatment of cartilage injuries is the administration of SPRF to the site of cartilage damage. This method should be preceded by a diagnosis of the cartilage injury. Very seriously damaged cartilage may not be successfully treated by this technique. Also, some healthy cartilage may be necessary to be present.

The SPRF is prepared as described herein, and included into an appropriate physiologically acceptable buffer system. In an embodiment the SPRF

Matrix (Scaffold) Assisted Administration of SPRF

In an embodiment SPRF can be added to a matrix analogously to a matrix-induced autologous chondrocyte implantation matrix, with or without chondrocytes.

The foregoing description will be more fully understood with reference to the following examples. Such examples are, however, merely representative of methods of practicing one or more embodiments of the present invention and should not be read as limiting the scope of invention.

### EXAMPLES

### Example 1: Platelet-Rich Plasma as an Adjuvant Therapy in Aseptic Femoral Head Necrosis

In a retrospective clinical observational study two surgical treatments were compared for avascular femoral head necrosis. Patients of the control group (n=13) were treated with core decompression alone, in the PRP group (n=19) core decompression was completed with the impaction of autologous bone chips mixed with autologous PRP. hi the clinical observational study six years after the operation the PRP group had significantly lower failure rate (21% vs 67%, p<0.05) indicated by prosthesis implantation.

However, the exact role and cellular mechanisms are unknown and further data are necessary to prove the effect of the method.

### Example 2: Preparation of an SPRF Composition, which is an Exemplary Serum Fraction of the Invention

A preparation was prepared which was free of platelets, however was rich in platelet-derived factors. The description of the procedure applied is as follows:

1. Venous blood was drawn into a standard, native tube without any additives.

2. Spinned it down instantly, preferably within 3 minutes, in a centrifuge at 1600-1700 G, for 5-10 minutes.

3. The red blood cells were collected at the bottom of the tubes, a yellowish coagulum floats on top of the red blood cell fraction in clear plasma. This clot (coagulum or coagel) was removed with a forceps and put on a clean petri dish.

4. The clot was gently squeezed to obtain the fluid out of the clot: The fluid obtained from the clot is essentially the final SPRF composition. As an estimate 0.4 ml final product can be gained from 6 ml of blood.

In order to speed up the clotting mechanism a silica-coated blood collection tube or a glass tube can also be used for drawing blood.

### Example 3: Bone Explants and Oxygen Glucose Deprivation (OGD)

In this in vitro study, bone samples were obtained from the removed femoral head during total hip replacements for primary osteoarthritis. Femoral heads were obtained from patients suffering from coxarthrosis and undergoing hip replacement surgery, during which the femoral head is extracted in its entirety and discarded as surgical waste.
Average 0,004 g weight explants (n=40 pieces/patient) were harvested from the femoral heads
The explants were transported into cell culture conditions at 37° C. in Dulbecco's Modified Eagle Medium containing 1 g/l glucose, 5% Penicillin-streptomycin and 10% fetal bovine serum (Stem cell medium).

After an incubation of 3 days of the femoral heads oxygen-glucose deprivation (OGD) was used to model the poor circulation of the femoral head. At a tissue level OGD models cellular damage and impaired regeneration which is characteristic for degenerative bone diseases such as aseptic necrosis, osteochondrosis, osteoarthrosis, etc. The femoral heads were placed into glucose and amino-acid free medium at an oxygen level of O₂<0.5 mmHg (replaced with N2 gas). The tests have been continued at 1, 2.5, 3.5, 4, 5, and 7 hours after which the normal cell culture conditions were restored.

For qualitative testing of cell viability live and dead cells were labeled with

Calcein-AM (488 nm) and Ethidium-Homodimer-2 (546 nm) fluorescent dyes, then evaluated by confocal microscopy (ZEISS LSM confocal microscopy, 20×).

For quantitative analysis of cell viability the methyl-thiazol-tetrasolium (MTT) assay was used with the following parameters: 1 h incubation in MTT solution, 1 h solubilization in isopropanol, absorbance measures at 570 and 690 nm₅ correcteted with the dry weight of bones. Assay was carried out at 37° C. In preliminary experiments, incubation was tested for 10 minutes, 1, 2, 5 hours, and solubilization in isopropanol was tested for 10 minutes, 1, 2, 3, 4, 5, 6, 20 hours.

### Example 4: Preparation of an Exemplary Serum Fraction of the Invention

(SPRF) And its Characterization

In an early variant of the method, Platelet-rich plasma was isolated by the double-centrifugation protocol. Blood from healthy adult donors was collected in EDTA tubes (BD Vacutainer®, K2E EDTA) and centrifuged at 1300 rpm (320 g) for 12 minutes. The supernatant was removed and centrifuged at 3000 rpm (1710 g) for 10 minutes. The pellet was resuspended in stem cell medium at a 1:4 ratio during the OGD therapy and after that. Heparinized PRP was created by adding 100 μl fractionated heparine (Clexane 4000 NE/0.4 ml) to 1200 μl PRP after the isolation. Platelet-rich fibrin was prepared by centrifugation without anticoagulants for 5 minutes at 3000 rpm (1710 g). A fibrinous gel was removed from the tube and the fluid gently squeezed out of the gel to obtain isolated SPRF, which was added to the stem cell medium in 1:10 ratio. The amount of serum was about 500 μl of final product from 6 ml of blood) and 2 ml of final product from 6 ml of blood

### Example 5: Effect of Serum Fractions on Bone Explants after Oxygen Glucose Deprivation

Bone explants were harvested from the discarded femoral heads from patients undergoing hip replacement. Bone grafts of about 10 mm³ were collected and transferred immediately into Dulbecco's Modified Eagle Medium containing 1 g/l of glucose, 1% penicillin-streptomycin, and 10% fetal bovine serum. The explants were cultured in this medium under standard cell culture conditions in 24-well plates. Oxygen-glucose deprivation (OGD) was performed in a Pecon incubation system (Erbach-Bach, Germany) on the third day after explantation. The bone pieces were transferred into stem cell medium lacking glucose and amino acids and the oxygen was flushed with nitrogen to 0.5% O₂ level for 7 hours. After completion of OGD the medium was replaced and the explants were cultured in 20% oxygen and 5% CO₂. Blood fractions were added to the medium in a ratio of 1:4 just before OGD and was refreshed at medium changes. Both PRP and SPRF was prepared fresh just before use and never stored or frozen.

The grafts were incubated in a 1:9 diluted mixture of 3-(4,5-Dimethyl-2-thiazolyl)-2,5-diphenyl-2H-tetrazolium bromide (MTT, #M5655, Sigma) and stem cell medium at 37° C. for 60 min then diluted with isopropanol. Absorbance of the solution was measured by a PowerWave XS spectrophotometer at 570 nm and noise was filtered out by measuring the absorbance at 690 nm. The MTT-assay was performed on the third and sixth days after OGD.

There were only a few living cells on the bone chips on the day of the operation, and these cells were damaged. The samples were obtained from different patients. To get the various bone chips into a similar state, they were incubated in stem cell medium at 37° C. and 5% CO₂ for 3 days. Sufficient number of cells were detected on the 3^{rd} day, therefore OGD was started to model the ischemic condition. Based on the data of four patients significant difference was shown by t-test between cell viability of the bone chips on the day of surgery and after 3 days of incubation (81.75±47.72 vs. 106.28±55.24).

To achieve the ischemic state OGD was applied for many different intervals. Bone samples were observed for one, two and a half, three and a half, five and seven hours of OGD. After the OGD treatment lasting 5 hours, cell viability of the OGD treated and untreated groups were determined by MTT assay and it was found that 5 hours of treatment is not enough to damage cells (n=12 explants/groups, control: 50.36±6.66 vs. OGD: 36.97±3.00, t-test, not significant). Before OGD healthy adherent cells could be seen in green, and with increasing the time of OGD to 7 hours these cells lost their branches, changed their shape, got damaged or killed, so their color turned red. Significant difference was shown between the control group and the OGD-treated group by our quantitative measurement.

During the PRP treatment explants of the treated group received a mixture of PRP and stem cell medium in a 1:4 ratio.

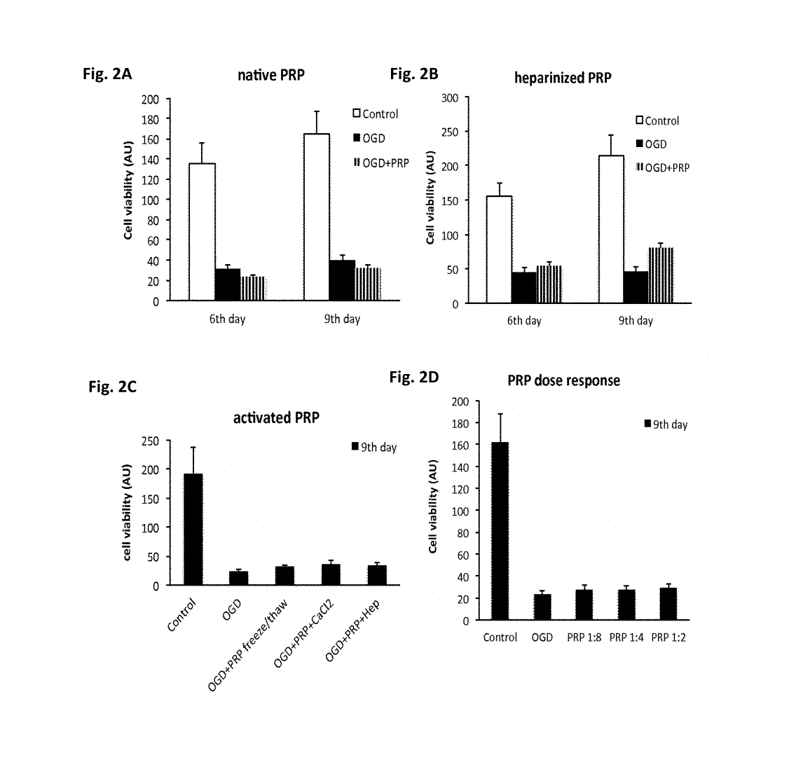

PRP cannot improve the viability of the cells after the ischemic condition (FIGS. 2A-D). As PRP was put into the stem cell medium, red blood cells in PRP were coagulated and the consistence of the solution became jelly-like. To avoid this consistency heparin was put into the PRP solution and cell viability was examined based on the protocol described earlier. A tendential growth was observed on the 6th day after the OGD compared to the untreated group, but this was not significant. Significant difference was not observed after testing the PRP in higher and lower concentrations (FIGS. 2A-D). To activate platelets, three methods were tried: freeze and thaw, added CaCA or CaCA and thrombin, but none of them changed the earlier results (FIGS. 2A-D).

The effect of SPRF during the OGD was examined. Treated explants were incubated in stem cell medium containing SPRF 1:4 scale for 7 hours. Based on the result of MTT assay, it is concluded that the group treated with SPRF did not have higher cell viability compared to the untreated group. SPRF cannot protect the immediate, acute effect of OGD (Data from 2 patients, control group: 70.18±6.64, OGD group: 24.85±2.49, SPRF group: 26.78±3.49, not significant difference).

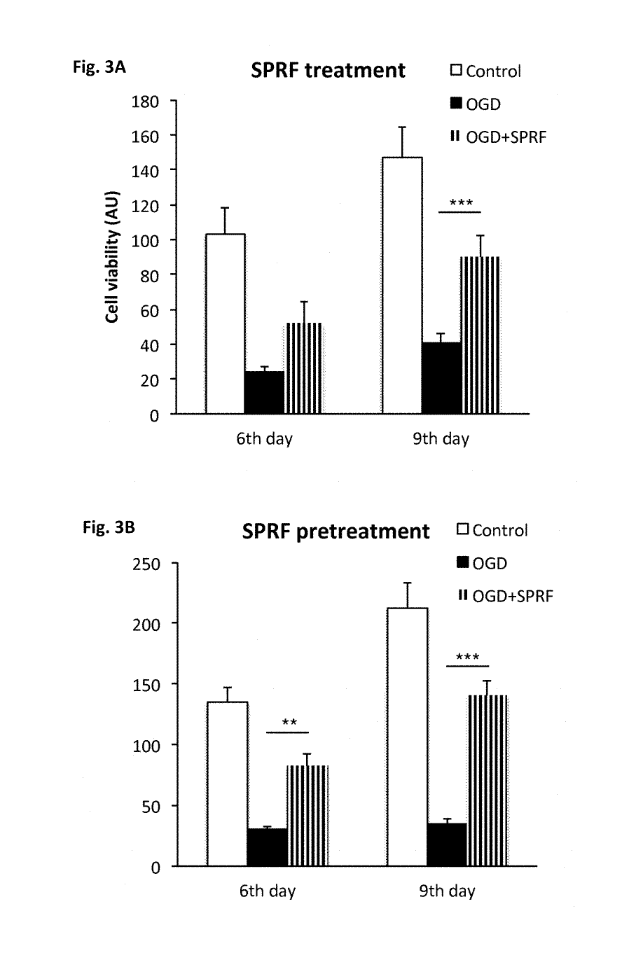

After that long-term effect of SPRF was examined. Explants were treated during OGD and continuously for 6 days after OGD. On the 3rd day the medium was changed and cell viability assay was done and tendencial growth was shown in the SPRF-treated group. After another 3 days of incubation after the OGD the difference was significant (FIGS. 3A-B).

In the pre-treated groups explants have received SPRF from the day of the surgery. In these cases the positive effect of SPRF can be declared, because significant difference was already detected between the treated and untreated groups after 3 days of OGD (4 patients, n=24 explants/groups, **:p<0.01), which difference was more pronounced on the 6th day after OGD (4 patients, n=24 explants/groups, ***:p<0.0001) (FIGS. 3A-B). In our study two rather similar blood fractions were compared in a model of bone ischemia. Unexpectedly, a positive effect from PRP could not be observed even at high concentrations, while SPRF significantly improved the proliferation capacity of osteoblast cells damaged by ischemia. It is also of note that the proliferative effect was additive to the effect of FBS, a normal constituent of stem cell culture media, and was only observed at the postischemic state.

### Example 6: Analysis of the Composition of Serum Fractions

For determination the growth factors and angiogenesis-related proteins in the SPRF and PRP Proteome Profiler Human Angiogenesis Array Kit (R&D System, #ARY 007) was applied and Adobe Photoshop was used for quantitation of protein expression. For the quantitative determination of platelets and ions in SPRF Sysmex XT 4000i and Beckman Coulter AU5800 was used. Results are reported as mean±SEM. Statistical significances were determined by t-test or one-way ANOVA with Tukey's post-hoc tests as appropriate with the Graphpad Prism software. Significance values of p<0.05 were considered significant.

TABLE 1
Laboratory parameters of blood fractions. Salts and proteins were measured by a
Sysmex XT 4000i device, cell counts were determined by a Beckman Coulter AU5800 device. Data is
presented as average ± SEM, n = 3.

| | | | | Normal range in | |
|---|---|---|---|---|---|
| | SPRF | PRP | PPP | whole blood | Unit |
| Sodium | 141.00 ± 0.58 | 138.33 ± 0.67 | 139.00 ± 1.00 | 133-146 | mmol/l |
| Potassium | 4.04 ± 0.19 | 4.38 ± 0.26 | 3.90 ± 0.19 | 3.5-5.3 | mmol/l |
| Calcium | 2.31 ± 0.01 | 2.33 ± 0.04 | 2.30 ± 0.02 | 2.12-2.57 | mmol/l |
| Magnesium | 0.84 ± 0.01 | 0.88 ± 0.04 | 0.83 ± 0.00 | 0.6-1.1 | mmol/l |
| Chloride | 105.67 ± 0.88 | 103.00 ± 0.58 | 102.67 ± 0.88 | 99-111 | mmol/l |
| Phosphor | 1.21 ± 0.08 | 1.31 ± 0.08 | 1.16 ± 0.08 | 0.87-1.45 | mmol/l |
| Glucose | 5.36 ± 0.32 | 4.61 ± 0.34 | 5.26 ± 0.3 | 3.6-6.0 | mmol/l |
| Total protein | 74.87 ± 1.53 | 75.77 ± 1.7 | 74.03 ± 2.4 | 60-80 | g/l |
| Albumin | 49.67 ± 0.07 | 48.27 ± 0.33 | 47.57 ± 0.82 | 35-52 | g/l |
| IgG | 12.04 ± 1.34 | 10.22 ± 1.17 | 12.95 ± 1.17 | 6.9-14 | g/l |
| Hemoglobin | 0.00 ± 0.00 | 6.33 ± 0.88 | 0.33 ± 0.33 | 115-155 | g/l |
| Fibrinogen | 0.00 ± 0.00 | 1.07 ± 0.09 | 0.00 ± 0.00 | 1.5-4 | g/l |
| Red blood cells | 0.00 ± 0.00 | 0.28 ± 0.04 | 0.00 ± 0.00 | 4.2-6.1 | T/l |
| White blood cells | 0.01 ± 0.01 | 14.85 ± 2.61 | 0.01 ± 0.01 | 4.8-10.8 | G/l |
| Platelets | 1.33 ± 0.33 | 242.33 ± 75.9 | 16.00 ± 4.93 | 150-400 | G/l |

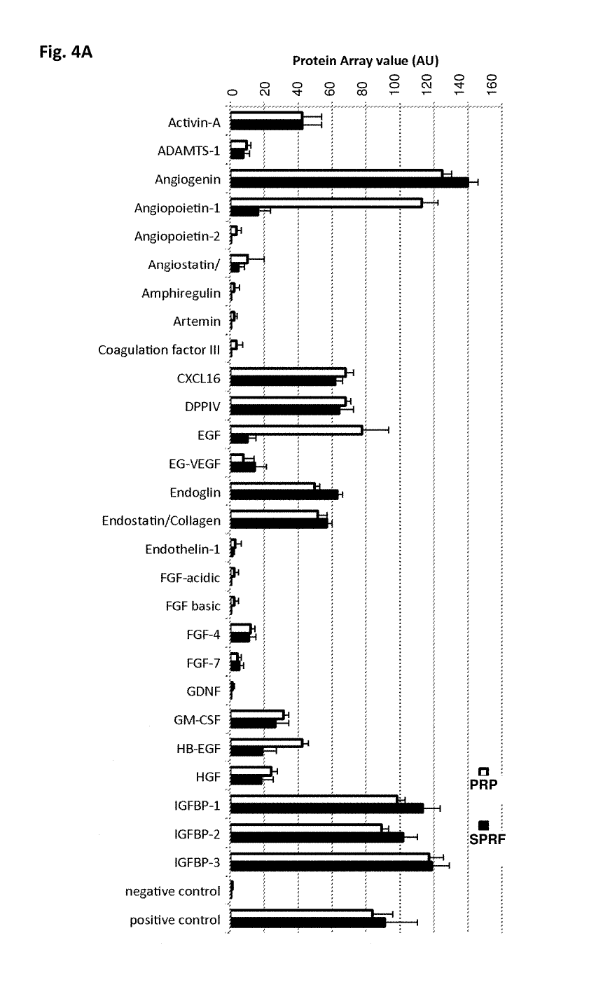

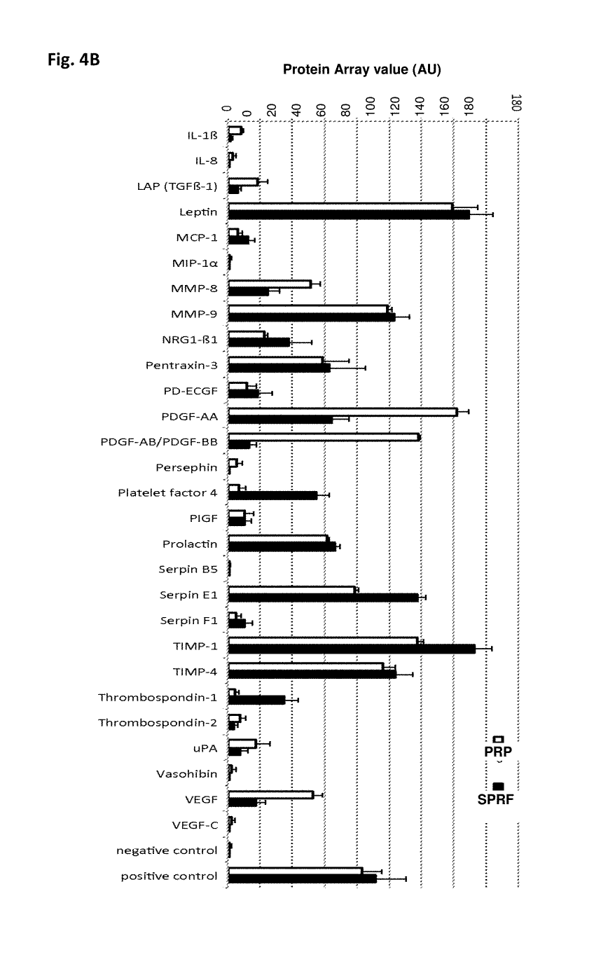

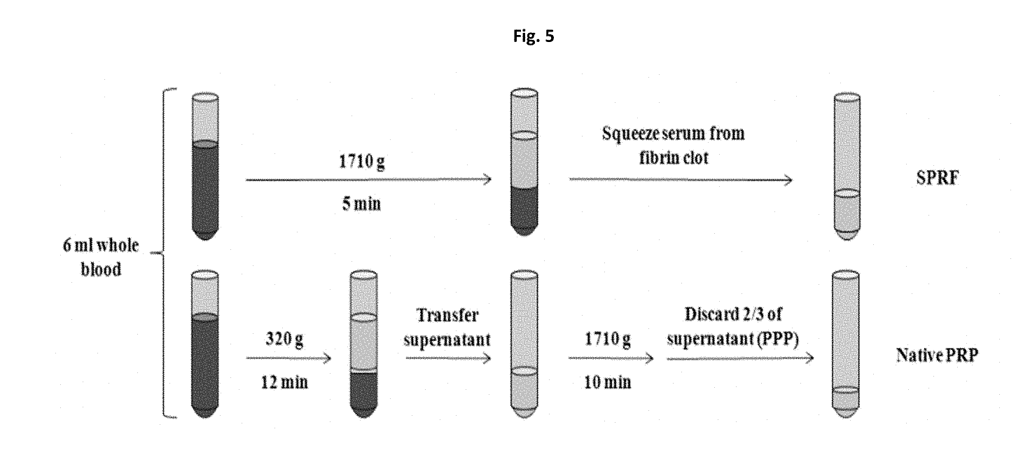

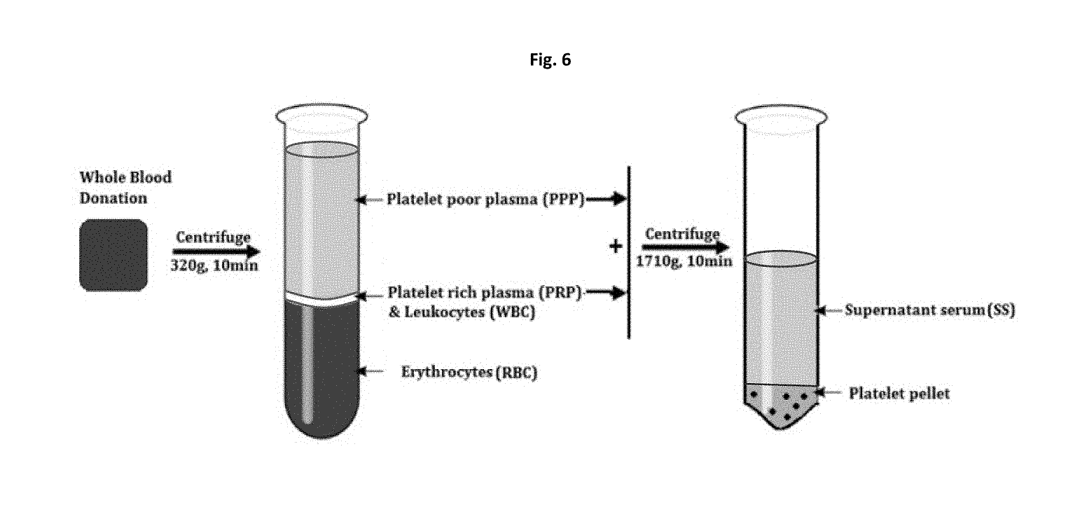

There are several key differences between PRP and SPRF measured by the proteome profiler assay (FIGS. 4A-B). The differences between the two fractions are limited to a handful of proteins, and it is a straightforward explanation that those factors that have higher levels in the SPRF fraction are responsible for the effect. It is noted that the full proteomics analysis of platelet release has just been compiled and it contains 3500 proteins, significantly more than those measured by the Proteome Profiler assay. However, two important proteins are often overlooked in these analyses: albumin and fibrin (Table 1). Both proteins are abundant in serum and have rather well-described effects on cell proliferation. It was previously described that the in vitro and in vivo bone inducting effects of serum albumin, which raises the possibility that albumin itself is the active factor in PRP and SPRF. However, since albumin is present in both PRP and SPRF at comparable levels, plus it is also added to the culture medium in the form of FBS, it is concluded that it would not be responsible for the effects of SPRF observed in the current study.

A clear difference between PRP and SPRF preparations is the presence of fibrin (Table 1). Fibrin or the inactivated form fibrinogen is the second most abundant protein in serum and is present in both native and heparinized PRP while it is missing in SPRF. Several studies described that fibrin has a very strong pro-inflammatory reaction by specifically activating macrophages. Fibrin is also known as a key factor in the bone healing process after a fracture as the first step of enchondral bone formation. Although not all details of the cellular connections of fibrin is clear, it is reasonable to hypothesize that it is at least partly responsible for the differences in the proliferative action of SPRF versus PRP. It is also of importance that the model used in the present study is not designed to mimic bone healing under normal conditions, but rather regeneration potential of a damaged tissue. While the inflammatory response during an acute injury of a broken healthy bone may be beneficial, it has an opposite effect in a degenerative tissue where the remodelling capacity of the cells is impaired. It is believed that the current model resembles this later situation by mimicking an ischemic period. The observation that serum fractions had no effect on the “healthy” state of the bone explants but in the postischemic period also supports the idea that the current model, with its limitations as an ex vivo system, more resembles degenerative bone tissues. Furthermore, since the bone stock was femoral heads explanted at total hip replacement procedures in end-stage osteoarthosis, the current results should be interpreted in this context.

It is concluded that isolating serum from platelet rich fibrin has unique regenerative properties in damaged bone tissues. The isolation of SPRF is a simple procedure which can be performed at the bedside, providing an autologous mix of growth factors which may even be used in degenerative bone diseases. The fact that SPRF is devoid of fibrin and has generally fewer constituents than PRP, but better effects in this specific case is a further step in the standardization of serum products. Based on the current ex vivo human study, the clinical translation of the use of SPRF is initiated in degenerative and ischemic bone diseases.

The short term safety of PRP is well-established by numerous clinical studies, however, concerns emerged regarding its efficacy. Attempts at compiling a meta-analysis face the problem of non-standardized nomenclature, diverse isolation protocols and treatment regimens. Even well-designed studies focusing on a niche indication struggle with the very high variation of growth factor levels in PRP. Since PRP is essentially a mixture of known and yet unknown active agents, it is not evident which can be used as a reference compound for dosing. Therefore, currently the best way of standardization is defining the product by the isolation protocol rather than its constituents.

### Example 7: SDF-1 Determination in SPRF

SDF-1 (Stromal cell-derived factor-1), also known as PBSF (pre-B-cell growth-stimulating factor), is a recently discovered protein belonging to the alpha chemokine (CXC) family of cytokines. SDF-1 alpha and SDF-1 beta are the first cytokines initially identified using the signal sequence trap cloning strategy from a human bone-marrow stromal cell line. SDF-1 has chemotactic activity on resting T lymphocytes and monocytes. The SDF-1 ELISA (Enzyme-Linked Immunosorbent Assay) kits [Sigma-Adrich, RAB0123, Human SDF 1 alpha ELISA Kit and RAB0124 SIGMA Human SDF-1 beta ELISA Kit] are in vitro enzyme-linked immunosorbent assays for the quantitative measurement of human SDF-1 in plasma (serum samples are not recommended for use in this assay as human SDF-1 concentration is low in normal plasma, it may not be detected in this assay), cell culture supernatants, and urine. This assay employs an antibody specific for human SDF-1 coated on a 96-well plate. Standards and samples are pipetted into the wells and SDF-1 present in a sample is bound to the wells by the immobilized antibody. The wells are washed and biotinylated anti-human SDF-1 antibody is added. After washing away unbound biotinylated antibody, HRP-conjugated streptavidin is pipetted to the wells. The wells are again washed, a TMB substrate solution is added to the wells and color develops in proportion to the amount of SDF-1 bound. The Stop Solution changes the color from blue to yellow, and the intensity of the color is measured at 450 nm. The standard dilution curve was prepared using the following SDF-1 concentrations (pg/ml): 6000, 3000, 1500, 750, 375, 187.5, 93.75.

### Example 8

Serum from platelet-rich fibrin (SPRF) and the platelet-rich plasma (PRP) were obtained from whole blood of 11 donors. Isolated SPRF and PRP will be stored at −80° C. as stocks and aliquots for cell-culture experiments and GF, cytokine quantification assays by Luminex

Materials

Sterile

Tweezers (sharp and thin tweezer), Sharp scissor

VACUETTE® 9 ml K3 EDTA Blood Collection Tube (REF. 455036, Greiner Bio-one)

VACUETTE® 9 ml Z Serum C/A (REF.no. 455092, Greiner Bio-one)

Polypropylene falcon tubes 15 ml

Eppendorf tubes 2 ml

Non-Sterile

Centrifuge

Method

Isolation of SPRF:

a) Whole blood obtained from donors was centrifuged at 1700 g for 10 mins at RT in the VACUETTE® 9 ml Z Serum C/A.

b) The fibrin clot from the tube was gently removed with a sharp tweezer and placed onto a sterile petri dish and the red blood cells at the bottom of the fibrin clot were cut with a sharp scissor and discarded.

c) Now, using a flat forceps the lysate was squeezed out of the fibrin clot, collected, stored at −80° C.

Isolation of PRP:

d) Whole blood obtained from donors was centrifuged at 320 g for 12 mins at RT in the VACUETTE® 9 ml K3 EDTA blood collection tube.

e) Three layers were formed in the collection tube. A bottom layer containing red blood cells, middle layer containing the buffy coat and top layer containing Platelet-Poor plasma (PPP).

f) The top layer (PPP) was aspirated along the middle layer (buffy coat) and transferred into a 15 mL falcon tube and centrifuged at 1700 g for 10 mins, the pellet was resuspended into an corresponding volume to the isolated SPRF in the supernatant (PPP), stored at −80° C.

Data from Informed Consent and Parameters for Isolation

TABLE 2.0
Isolation of SPRF with defined parameters

| | | | | | | | | | Volume |
|---|---|---|---|---|---|---|---|---|---|
| | | | | | | | | | *vials |
| | | | | Volume | | | | | stored |
| | | | | *tubes | Centrifugation | | Collected | Volume | at −80° C. |
| Donor ID | | | Total blood | centrifuged | period for | G- | serum from | stored at −80° C. | for |
| No. | Gender | Age | collected | for SPRF | SPRF | Force/RPM | PRF | for | Luminex |
| Donor 1 | F | 57 | 36 mL | 9 mL* 2 | 10 mins | 1710 g/3000 | 1 | 1 mL | NIL |
| Donor 2 | F | 40 | 36 mL | 9 mL* 2 | 10 mins | 1710 g/3000 | 1.5 | 1.5 mL | NIL |
| Donor 3 | F | 24 | 36 mL | 9 mL* 2 | 10 mins | 1710 g/3000 | 0.2 | 0.2 mL | NIL |
| Donor 4 | M | 25 | 36 mL | 9 mL* 2 | 10 mins | 1710 g/3000 | 1.8 | 1.8 mL | NIL |
| Donor 5 | M | 32 | 36 mL | 9 mL* 2 | 10 mins | 1710 g/3000 | 2.4 | 2.4 mL | NIL |
| Donor 6 | M | 34 | 36 mL | 9 mL* 2 | 10 mins | 1710 g/3000 | 2.5 | 2.5 mL | NIL |
| Donor 7 | M | 29 | 36 mL | 9 mL* 2 | 10 mins | 1710 g/3000 | 1.1 | 1.1 mL | NIL |
| Donor 8 | F | 31 | 36 mL | 9 mL* 2 | 10 mins | 1710 g/3000 | 1 | 1.0 mL | NIL |
| Donor 9 | F | 39 | 36 mL | 9 mL* 2 | 10 mins | 1710 g/3000 | 1.05 | 0.8 mL | 75 ul*2 |
| Donor 10 | M | 30 | 36 mL | 9 mL* 2 | 10 mins | 1710 g/3000 | 2.65 | 2.5 mL | 75 ul*2 |
| Donor 11 | F | 36 | 36 mL | 9 mL* 2 | 10 mins | 1710 g/3000 | 2.15 | 2.0 mL | 75 ul*2 |
| Donor 12 | M | 36 | 36 mL | 9 mL* 2 | 10 mins | 1710 g/3000 | 1.45 | 1.3 mL | 75 ul*2 |
| Donor 13 | F | 58 | 36 mL | 9 mL* 2 | 10 mins | 1710 g/3000 | 1.625 | 1.5 mL | 125 ul*1 |
| Donor 14 | F | 53 | 36 mL | 9 mL* 2 | 10 mins | 1710 g/3000 | 3.725 | 3.6 mL | 125 ul*1 |
| Donor 15 | F | 52 | 36 mL | 9 mL* 2 | 10 mins | 1710 g/3000 | 2.425 | 2.3 mL | 125 ul*1 |
| Donor 16 | F | 58 | 36 mL | 9 mL* 2 | 10 mins | 1710 g/3000 | 1.0 | 0.875 mL | 125 ul*1 |
| (not sterile) | | | | | | | | | |
| Donor 17 | F | 27 | 36 mL | 9 mL* 2 | 10 mins | 1710 g/3000 | 1.8 | 1.675 mL | 125 ul*1 |
| Donor 18 | F | 50 | 36 mL | 9 mL* 2 | 10 mins | 1710 g/3000 | 2.3 | 2.175 mL | 125 ul*1 |
| Donor 19 | M | 57 | 36 mL | 9 mL* 2 | 10 mins | 1710 g/3000 | 2.0 | 1.875 mL | 125 ul*1 |
| Donor 20 | M | 61 | 36 mL | 9 mL* 2 | 10 mins | 1710 g/3000 | 2.4 | 2.275 mL | 125 ul*1 |

TABLE 3.0
Isolation of PRP with defined parameters

| | | | | | | | | | | Vol. | | Volume* |
|---|---|---|---|---|---|---|---|---|---|---|---|---|
| | | | | | | | | | | stored | | vials |
| | | | | Volum. | | | | | | −80° C. | | stored at |
| | | | Total | *tubes | Centrif. | | Coll. | Centrif. | | for | | −80° C. |
| Donor | | | blood | centrifuged | period 1 | G- | S. nat. | period 2 | G- | Stock | | for |
| ID No. | Gender | Age | collected | for PRP | (mins) | Force/RPM | (mL) | (mins) | Force/RPM | (mL) | | Luminex |
| Donor 1 | F | 57 | 36 mL | 9 mL* 2 | 12 | 320/1200 | 4 | 10 | 1710/3000 | 1 | | NIL |
| Donor 2 | F | 40 | 36 mL | 9 mL* 2 | 12 | 320/1200 | 6 | 10 | 1710/3000 | 1.5 | | NIL |
| Donor 3 | F | 24 | 36 mL | 9 mL* 2 | 12 | 320/1200 | 4 | 10 | 1710/3000 | 0.2 | | NIL |
| Donor 4 | M | 25 | 36 mL | 9 mL* 2 | 12 | 320/1200 | 4 | 10 | 1710/3000 | 1.8 | | NIL |
| Donor 5 | M | 32 | 36 mL | 9 mL* 2 | 12 | 320/1200 | 6 | 10 | 1710/3000 | 2.4 | | NIL |
| Donor 6 | M | 34 | 36 mL | 9 mL* 2 | 12 | 320/1200 | 6 | 10 | 1710/3000 | 2.5 | | NIL |
| Donor 7 | M | 29 | 36 mL | 9 mL* 2 | 12 | 320/1200 | 4 | 10 | 1710/3000 | 1.1 | | NIL |
| Donor 8 | F | 31 | 36 mL | 9 mL* 2 | 12 | 320/1200 | 6 | 10 | 1710/3000 | 1 | | NIL |
| Donor 9 | F | 39 | 36 mL | 9 mL* 2 | 12 | 320/1200 | 6 | 10 | 1710/3000 | 0.8 | | 75 ul*2 |
| Donor 10 | M | 30 | 36 mL | 9 mL* 2 | 12 | 320/1200 | 6 | 10 | 1710/3000 | 2.5 | | 75 ul*2 |
| Donor 11 | F | 36 | 36 mL | 9 mL* 2 | 12 | 320/1200 | 4 | 10 | 1710/3000 | 2.5 | | 75 ul*2 |
| Donor 12 | M | 36 | 36 mL | 9 mL* 2 | 12 | 320/1200 | 4 | 10 | 1710/3000 | 1.3 | | 75 ul*2 |
| Donor 13 | F | 58 | 36 mL | 9 mL* 2 | 12 | 320/1200 | 4 | 10 | 1710/3000 | 1.5 | | 125 ul*1 |
| Donor 14 | F | 53 | 36 mL | 9 mL* 2 | 12 | 320/1200 | 4 | 10 | 1710/3000 | 3.6 | | 125 ul*1 |
| Donor 15 | F | 52 | 36 mL | 9 mL* 2 | 12 | 320/1200 | 6 | 10 | 1710/3000 | 2.3 | | 125 ul*1 |
| Donor 16 | F | 58 | 36 mL | 9 mL* 2 | 12 | 320/1200 | 6 | 10 | 1710/3000 | 0.875 | | 125 ul*1 |
| (not | | | | | | | | | | | | |
| sterile) | | | | | | | | | | | | |
| Donor 17 | F | 27 | 36 mL | 9 mL* 2 | 12 | 320/1200 | 6 | 10 | 1710/3000 | 1.675 | | 125 ul*1 |
| Donor 18 | F | 50 | 36 mL | 9 mL* 2 | 12 | 320/1200 | 6 | 10 | 1710/3000 | 2.175 | mL | 125 ul*1 |
| Donor 19 | M | 57 | 36 mL | 9 mL* 2 | 12 | 320/1200 | 6 | 10 | 1710/3000 | 1.875 | mL | 125 ul*1 |
| Donor 20 | M | 61 | 36 mL | 9 mL* 2 | 12 | 320/1200 | 6 | 10 | 1710/3000 | 2.275 | mL | 125 ul*1 |

*After centrifugation period 2, the pellet (PRP) was re-suspended in an equal amount of PPP (Supernatant) in a volume corresponding to the obtained volume from SPRF

Observations During Experiments
In the preparation of SPRF, it was noted that clot formation in a particular donor is dependent on the time factor; i.e., Whole blood fractions obtained from donors not centrifuged within 10, 5, 2 or preferably 1 minute(s) or immediately resulted in impaired clot formation.
In an example, we tested this observation, by obtaining two vials from a donor, that were centrifuged immediately (less than a minute) and one vial from the same donor, which was centrifuged after 5 minutes. It was observed that the vials centrifuged immediately (within 1 minutes) had a clot formation, whereas the vial centrifuged after 5 mins had no clot formation.
It has also been observed that the amount of PRF derived serum (quantity) isolated from each donor is donor dependent. It varies from each individual.

### Example 9—the Proliferative Effect of Serum Derivatives on Isolated Human Chondrocytes Obtaining and Culturing Chondrocytes

Human osteoarthritic articular cartilage was obtained from patients (n=3; age 60-80 years) undergoing total knee arthroplasty. For chondrocyte isolation, articular cartilage was minced into 2 mm3 pieces prior to enzymatic digestion with Liberase Blendzyme 3 (0.2 WU/mL, Roche Diagnostics GmbH, Mannheim, Germany) in medium (GIBCO DMEM/F12 GlutaMAX-I, Invitrogen, LifeTech Austria, Vienna, Austria) with antibiotics (penicillin 200 U/mL; streptomycin 0.2 mg/mL, and amphotericin B 2.5 μg/mL (Sigma-Aldrich Chemie GmbH, Steinheim, Germany)) under permanent agitation for 18 to 22 hours at 37° C. Subsequently, cell suspensions were passed through a 40 μm filter (BD, Franklin Lakes, N.J.) to remove debris, washed with phosphate-buffered saline (PBS), centrifuged (10 minutes, 500×g, room temperature) and resuspended in PBS. Cells were seeded into 75 cm2 culture flasks (Nunc, Rochester, N.Y.) at a density of 100 cells/cm2 and further cultivated in medium supplemented with antibiotics (see above), 10% fetal calf serum (PAA Laboratories GmbH, Linz, Austria), and 1-ascorbic acid (50 μg/mL; Sigma-Aldrich Chemie GmbH, Steinheim, Germany) at 37° C. in a humid environment with 5% CO2. Medium was changed every 3 days. For passaging, cells grown to 80% confluency were harvested by use of accutase (1.5 mL/flask; PAA Laboratories GmbH, Linz, Austria) counted, and seeded again (all experiments were performed in passage 1)

Proliferation Assay

OA chondrocytes (passage 1) were seeded in three 96-well plate (2000 cells/well) For obtaining reliable results, cells for every tested group were seeded in 20 wells (in few different rows placed on the 96-well plate—to eliminate any disruptions in the data that could be associated with the possible mistake in pipetting, resulting in differences with the number of cells in wells)—and for each group 12 wells should remain without cells only with media (to check the absorbance of medium). After seeding the cells were fed for 48 hours with 100 μl of standard growing medium (described above). 48 hours after seeding (day 0) standard growing medium was replaced with 100 μl of following growth medium per well: group with SPRF-DMEM medium (GIBCO®DMEM/F12 GlutaMAX™-I, Invitrogen, Vienna, Austria) with 10% SPRF, 2% Penicilin/Streptomycin, 1% Amphotericin; group with PRP-DMEM medium (GIBCO®DMEM/F12 GlutaMAX™-I, Invitrogen, Vienna, Austria) with 10% PRP, 2% Penicilin/Streptomycin, 1% Amphotericin, Heparine (2 U/ml); FCS group: standard growing medium as described above; serum free group (SF): DMEM medium (GIBCO®DMEM/F12 GlutaMAX™-I, Invitrogen, Vienna, Austria) with 2% Penicilin/Streptomycin, 1% Amphotericin. Experiments lasted 8 days and measurements were taken at three different time points (days 0, 4, 8—1 plate was used at one time point). Metabolic activity and proliferation rate of OA chondrocytes were investigated through XTT assay (Roche, Mainheim, Germany) according to the manufacturer's instructions. Relative fluorescence was measured using plate reader The BioTek's Synergy 2.

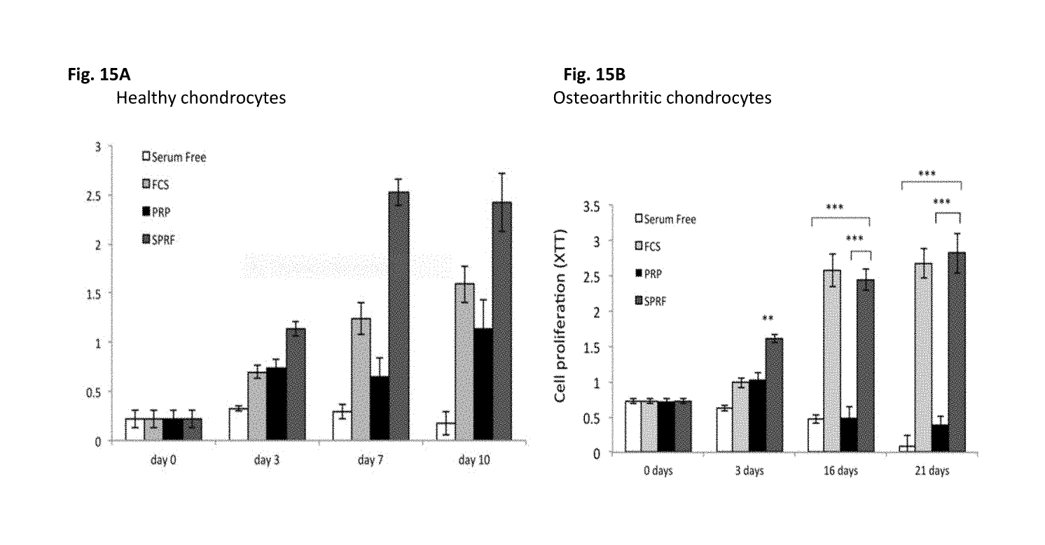

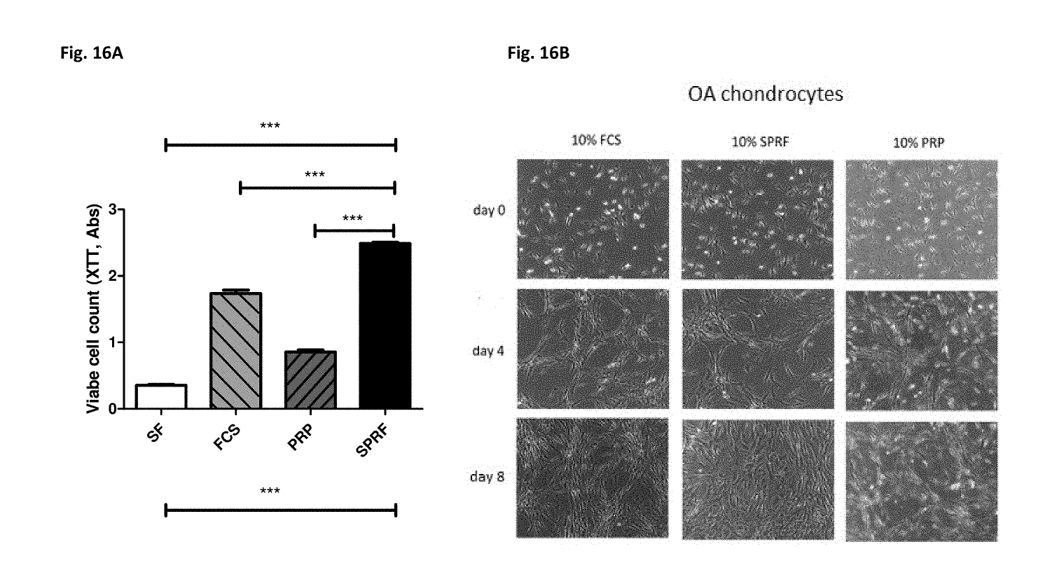

In a preliminary experiment, FIG. 15A and FIG. 15B, the proliferative effect of serum derivatives is shown in isolated human chondrocytes.

In FIG. 16A the mean results from 3 different experiments with XTT assay after 8 days is shown, performed in 96-well cell culture plate based monolayer culture with 2000 cells/well seeded, cultured in serum free DMEM (SF) or DMEM media supplemented with 10% SPRF, 10% PRP or 10% FCS. In FIG. 16B pictures of OA chondrocytes culture after 0, 4, 8 days with different media supplementation are shown.

Cells were harvested from either healthy or osteoarthritic human knee hyalin cartilage and grown in culture. In terms of proliferation support, FCS and SPRF are comparably effective, while PRP has no effect in healthy chondroytes. In osteoarthritic chondrocytes the differences are even more pronounced, SPRF outperforms FCS and PRP shows some effect.

### Example 10—SPRF Preparation in a Syringe Ready for Intraarticular Injection

18 mL blood (without anticoagulant) from the right or left forearm vein of the patient is taken using e hypodermic needle (e.g. butterfly needle) into glass or silica-coated tubes then spun in a centrifuge at 1714 g for 8 min (5-10 min). The red blood cell fraction is removed and the remaining yellowish clotted plasma (=platelet rich fibrin, PRF) is gently squeezed to harvest the SprF fraction. Alternatively, SPRF can be prepared in the hypACT Inject Auto medical device using the same method. The SPRF fraction is then transferred into a standard 5 mL syringe ready for intraarticular injection.

### Example 11—Autologous Chondrocyte Implantation

It is confirmed that the lesion is eligible for autologous chondrocyte implantation. Then a biopsy is taken from the cartilage and is sent for chondrocyte culturing (cell proliferation) in the laboratory. Chondrocyte proliferation is carried out as described above.

Cell implantation is performed in the following stage, consisting of arthrotomy, preparation of the chondral defect, harvesting of periosteum, hermetic fixation of periosteum over the lesion with stitches and fibrin glue, injection of chondrocyte concentrate and closing of the operative wound.

In a further variant, second generation ACI is applied, i.e. after cell expansion in a monolayer, the cells are deposited on a carrier membrane/matrix, obtaining a membrane sown with MACI® (chondrocytes (Verigen AG, Leverkusen, Germany).

In a further variant of the example, a third generation of ACI is applied, i.e the chondrocyte culture is deposited on a matrix of hyaluronic acid structured in three dimensions (Hyalograft-C®, Fidia Advanced Biopolymers, Abano Term, Italy), thus enabling homogeneous distribution of the chondrocytes inside the lesion.

Conclusions of Results with Chondrocytes

SPRF increases the proliferation rate of OA chondrocytes as observed with PRP on 2D substrates but is very donor dependent. However, SPRF does not redifferentiate the OA chondrocyte from the dedifferentiated state either under normoxic and hypoxic (physiological) conditions. However, PRP enhances proliferation & redifferentiation both under normoxic and hypoxic conditions from 24h to 72h.

Moreover, as an example, over a culture period of 9 days SPRF from younger donors (±35 years) reached a higher proliferation rate compared to older donors (±55 years). In contrast to the biological activity, growth factor concentrations (PDGF-BB, Leptin) were not age-dependent in the SPRF preparations. However, the growth factor concentrations of individual SPRF donors measured at two different time points were highly variable as quantified with a multiplex screening array. Our results indicate that SPRF from younger donors expedite proliferation of OA chondrocytes derived from older patients and can be a relevant serum replacement during cell culture expansion or in vivo therapy.

We have also found that FCS does not retain the redifferentiated state (Col2/Col1 differentiation index) under normoxic/hypoxic conditions from 24h to 72h.

Thus, SPRF increased the proliferation rate of dedifferentiated, preferably osteoarthritic chondrocytes to a larger extent than PRP, and also to a larger extent than FCS.

Moreover, proliferation and differentiation of chondrocytes can be separated and therefore a more regulated handling of the procedure. The inventors have also noticed that the effect of SPRF obtained from younger donors is stronger than dose obtained from elder donors.

### Example 12—Cell and Tissue Cultures for Experiments with Mesenchymal Stem Cells (MSCs)

All tissue culture procedures were carried out in a sterile laminar flow tissue culture hood. Cells and ex vivo explant cultures were maintained in an incubator at 37° C. and 5% CO₂ and 95% of humidity. hMSC proliferation assay

Cells were seeded in standard growing medium into 5 parallel wells of a 96-well plate (2000 cells/well). Cell-free wells were used as background control. 48 hours after the start of the incubation standard growing medium was refreshed only in a group (same medium type was kept), in the others the 10% (v/v) FCS supplement was changed for 10% (v/v) FCS+bFGF, or 10% (v/v) platelet rich plasma (PRP), or 10% (v/v) serum from platelet rich fibrin (SPRF). PRP-supplemented medium contained 2 U/mL heparine (Clexane, Sanofi Aventis, Paris, France). As negative control, serum-free medium was used. Following 2 and 5 days incubation cell viability assay was performed.

Human Mesenchymal Stem Cell (hMSC) 2D Culture Human mesenchymal stem cells (hMSCs) purchased from LONZA were seeded at 5000 cells/cm² in normal T-75 tissue culture flasks and maintained in standard growing medium: Dulbecco's modified Eagle's medium (DMEM), high glucose, GlutaMAX™ Supplement, pyruvate (Gibco, Paisley, Scotland), supplemented with 10% (v/v) fetal calf serum (FCS, Gibco, Paisley, Scotland), fibroblast growth factor (bFGF) 1 ng/ml (Sigma-Aldrich, St. Louis, USA), 2% Penicillin/Streptomycin (Sigma-Aldrich, St. Louis, USA) and 1% Amphotericin (Sigma-Aldrich, St. Louis, USA). Cell culture medium was refreshed twice a week.

Cell Culture Conditions for hMSCs

Four different media were used in the course of the experiments with hMSCs. (1) Basal medium: DMEM, high glucose, GlutaMax™ Supplement, pyruvate containing 10% FBS, 2% penicillin/streptomycin and 1% amphotericin. (2) Serum-free medium: DMEM, high glucose, GlutaMax™ Supplement, pyruvate (Gibco, Paisley, Scotland), containing 2% penicillin/streptomycin and 1% penicillin/streptomycin. (3) SPRF-medium: DMEM, high glucose, GlutaMax™ Supplement, pyruvate containing 10% SPRF, 2% penicillin/streptomycin and 1% amphotericin. (4) PRP-medium: DMEM, high glucose, GlutaMax™ Supplement, pyruvate containing 10% PRP, 2% penicillin/streptomycin and 1% amphotericin. Media were changed every 48 hours (support for 2 days injection). Cells were incubated in normal cell culture conditions (5% CO2, humidified atmosphere, 37° C.). 2000-3000 cells/well were seeded in 5 parallel wells for all sample type and for all time point. Percentage ratios are given in respect of the total volume.

Isolation and Culture of Human Subchondral Bone Pieces (Bone Explants; hSBEs)

hSBEs, 2 mm in diameter were harvested from patients undergoing total hip replacement surgery at the Orthopedic Clinics of Semmelweis University (Budapest, Hungary). All procedures were performed with permission of hungarian Ethical Committee. The donors had osteoarthritis, otherwise they were diagnosed not to have cancer, or any infectious or autoimmune disease. Only tissue that would have otherwise been discarded was used.

In an embodiment femoral heads (those would have been otherwise discarded) are sawn off that results in intense cell death at the sawn surface due to friction based heat shock. Therefore, bone explanted from the body was cut in half with a bone chisel and hSBEs were picked from the cut surface with a small chisel.

Explants were delivered to the laboratory in the same medium that was used for hMSC culture.

All further experiments were started following 48 hours of preincubation in the medium described above.

In an embodiment tissue cultures were maintained in basal medium (DMEM containing 10% FBS and 1% penicillin/streptomycin) at 37° C. and 5% CO₂ in a humidified atmosphere. After 2 days incubation samples were used for experiments with special media or harvested for RNA.

### Example 13—Viability Test for Cell Culture and Bone Explants

Cell viability of hMSCs and hSBEs was determined using Cell Proliferation Kit II (XTT; Roche, Mannheim, Germany) according to the manufacturer's instructions. Absorbance were measured after 4 hours incubation in the staining solution using a PowerWave Microplate Spectrophotometer (BioTek, Winooski, Vt., USA) at 480 nm with a reference wavelength at 650 nm. In case of hSBEs, bone pieces were removed from the labeling mixture right before absorbance measurement. Results were normalized with the weight of the chips considering that the bone size is proportional to the number of active cells.

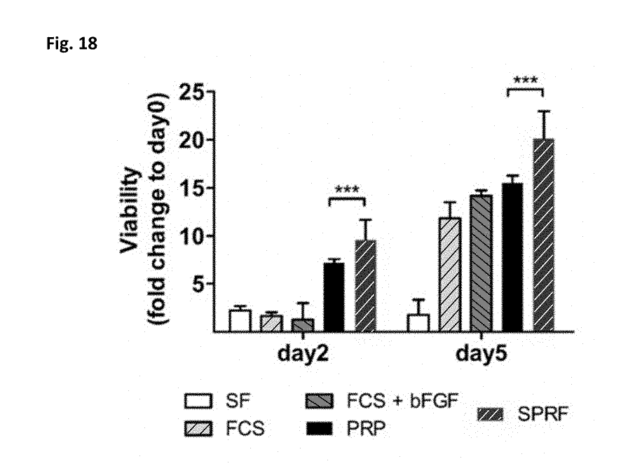

On FIG. 18. growth for the hMSC cells grown with FCS+bFGF or with FCS, only SPRF or PRP and without serum on plastic surface are shown. On the first day 2000-3000 cells were seeded pro well into a 96-well plate. Viability of cells was measured by XTT viability test. The viability on the 5^{th} day in case of cells supplemented with FCS+bFGF showed OD₄₅₀=1.428±0.064 absorbance values, only FCS supplemented OD₄₅₀=1.306±0.069, only SPRF supplemented OD₄₅₀=1.458±0.098, only PRP supplemented OD₄₅₀=0.954±0.075. The values are the average of five parallel measurements. It can be seen that the medium supplemented with SPRF provided the best result in terms of cell viability.

Tissue Culture Conditions for hSBPs

Three different media were used in the course of the experiments with hSBPs (1) Basal medium: DMEM containing 10% FBS and 1% penicillin/streptomycin. (2) Serum-free medium: DMEM containing 1% penicillin/streptomycin. (3) SPRF-medium: DMEM containing 10% SPRF and 1% penicillin/streptomycin. Media were changed every 48 hours. Tissue cultures were incubated in normal cell culture conditions (5% CO2, humidified atmosphere, 37° C.).

Cell Viability Test of hMSCs and hSBPs

XTT (sodium 2,3-bis(2-methoxy-4-nitro-5-sulfophenyl)-5-[(phenylamino)-carbonyl]-2H-tetrazolium)) assay (Roche, Cell Proliferation Kit II) was performed to measure the viability of cells. XTT Labeling Mixture was added to the culture medium and bones or cell monolayers in 0.3 mg/ml final concentration on a 96-well plate and the plate was placed back into the incubator for 4 hours. Bone pieces were removed and absorbance was measured on 450 nm on a plate reader. In case of hSBPs absorbance values were normalized to the dry weight of the bone pieces.

### Example 14—RNA Extraction and Reverse Transcription

Total RNA was isolated from 5 bone pieces with TRIzol Reagent (Ambion) following homogenization with liquid nitrogen in a mortar. 500 μl of TRIzol reagent was added to the homogenized tissue. Following 5 min incubation on room temperature centrifugation was carried out (12000 g, 1 minute, room temperature) to remove particulate debris from homogenized samples. The supernatant was transferred into a new tube and RNA was purified with Direct-zol™ RNA MiniPrep Kit (Zymo Research). RNA was eluted in 40 μl diethylpyrocarbonate-treated (DEPC) water. Measurement of RNA yield was performed by agarose gel electrophoresis and using a NanoDrop 1000A Spectrophotometer (Thermo Fisher Scientific, Waltham, Mass., USA). The purity of RNA was accepted, when values A260/280>2.0 and A260/230>2.0 were measured. Reverse transcription was performed using the first-strand cDNA synthesis kit as instructed by the manufacturer (ReadyScript™, Sigma Aldrich), i.e. reverse transcription to synthesize first strand cDNA was carried out for 30 min at 42° C., primed with an oligo (dT) primer bearing a T7 promoter.

Quantitative PCR

Real-time quantitative PCR was performed using ABI for quantifying the expression of activated leukocyte cell adhesion molecule (ALCAM/CD166: Hs00977641_m1). Values were calculated using the comparative threshold cycle (Cₜ) method and normalized to GAPDH (Hs02758991_g1) expression. Values were expressed as the mean±SD. Experiments were performed at least three times. Statistical analysis was performed using one-way analysis of variance (ANOVA) with Tukey-Kramer Multiple Comparison post-test.

### Example 15

Assessment of MSC Proliferation on Bone Explants

hSBPs were incubated in basal medium two days long. On the second day their viability showed 48.267±15.626 (n=3). On this day medium was changed for fresh basal medium, serum-free medium or SPRF-medium. Viability values on the 4th day were 92.997±17.025 (n=19), 117.357±19.383 (n=24) and 187.527±18.814 (n=24) in serum-free medium, basal medium and SPRF-medium, respectively. Viability values on the 7th day were 77.711±21.734 (n=7), 199.02±27.367 (n=15) and 224.212±28.023 (n=15) in serum-free medium, basal medium and SPRF-medium, respectively. Statistically significant differences at p<0.05 were determined by one-way analysis of variance (ANOVA).

Quantitative reverse transcription-polymerase chain reaction analysis was used to evaluate the expression of the hMSC associated gene CD166 (ALCAM). Compared to the second day expression of CD166 (ALCAM) molecule expression was 2.2-times higher in case of basal medium, and 2.1-times higher in case of SPRF-medium. All expression values are normalized to the expression of GAPDH. Our SPRF supplemented medium was as effective as the one, which was supplemented with specific stem cell media (FBS supplemented basal medium), however SPRF is from human autologous origin. All expression values are normalized to the expression of GAPDH.

### Example 16—Isolation of SPRF

a) Whole blood obtained from donors was immediately centrifuged at 1700 g for 10 mins at RT in BD Vacutainer® Z.
b) The fibrin clot from the tube was gently removed with a tweezer and placed onto a sterile Petri-dish and the red blood cells at the bottom of the fibrin clot were cut with a sharp scissor and discarded.
c) The lysate was squeezed out of the fibrin clot, collected and stored at −20° C.

Isolation of PRP
a) Whole blood obtained from donors was centrifuged at 320 g for 12 mins at RT in the BD Vacutainer® 6 ml K2E (EDTA).
b) Three layers had been formed in the collection tube. The bottom layer containing the red blood cells, middle layer containing the buffy coat and the top layer containing the platelet-poor plasma (PPP).
c) PPP was aspirated along the middle layer and transferred into a 15 ml Falcon tube and centrifuged at 1700 g for 10 mins
d) The pellet was resuspended into a corresponding volume to the isolated SPRF in the supernatant. Stored at −20° C.

### Example 17—Mesenchymal Stem Cell Proliferation Assay and Cell Morphology

To examine cell proliferation in presence of different serum derivatives, subconfluent hMSC cultures were incubated for 2 or 5 days in serum-free DMEM (SF) or in DMEM supplemented either with FCS, FCS+bFGF, PRP or SPRF, 10% (v/v) each. Viability of the samples was measured with XTT assay on the 1^{st}, 2^{nd} and 5^{th} day of the experiment.

SF, FCS and FCS+bFGF had no mitogenic effect after 2 days incubation. In the presence of PRP and SPRF viability of cells was elevated 7.18-fold and 9.57-fold, respectively. This significant proliferation could be due to the human origin of the applied supplement.

In SF medium viability was not significantly higher as on day 2. In the period between the 2^{nd} similar viability to FCS+bFGF for the 5^{th} day, 11.83-fold and 15.53-fold, respectively. Clearly the strongest effect had SPRF in the medium, where the cell viability 20.1-fold higher was compared to the zero day. This effect is very remarkable considering that the official culture medium and a treatment with a growth factor were less effective (FIG. 18).

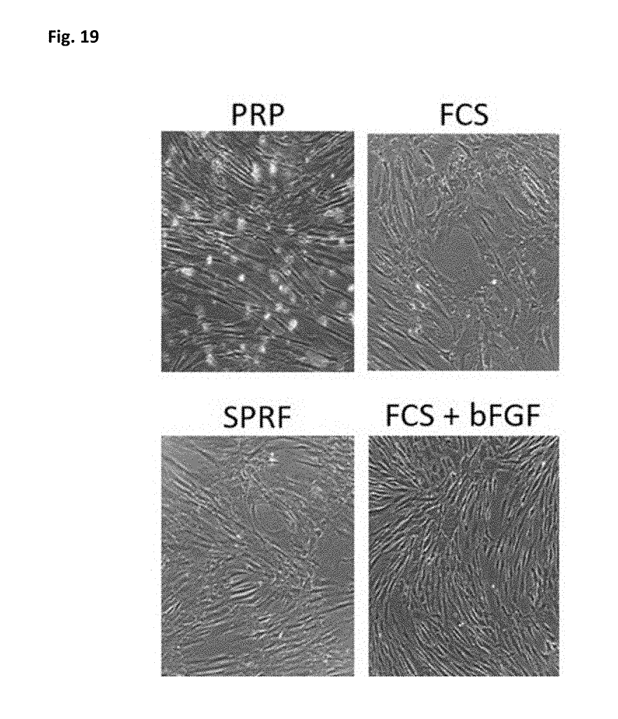

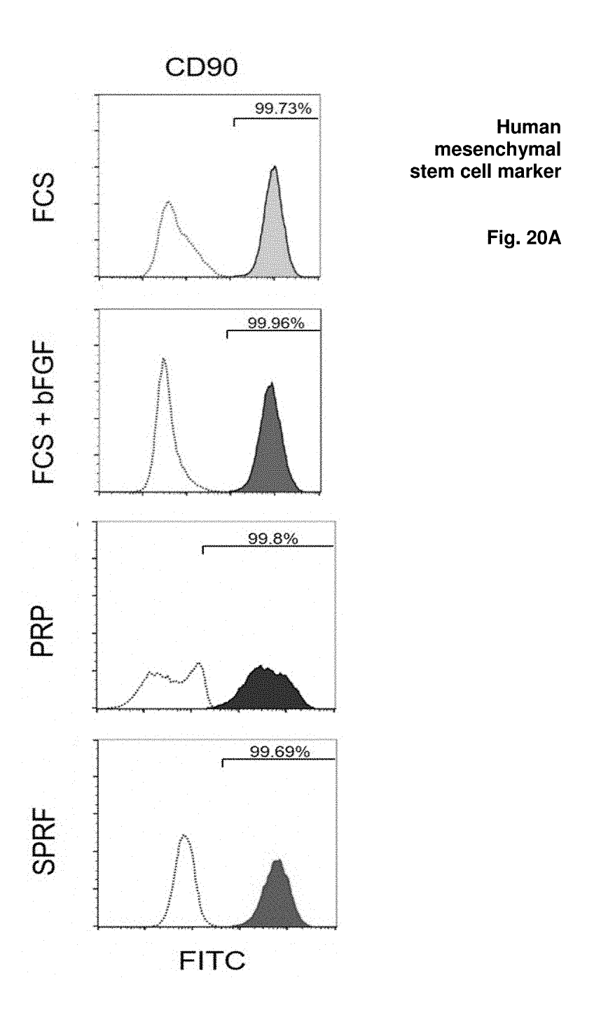

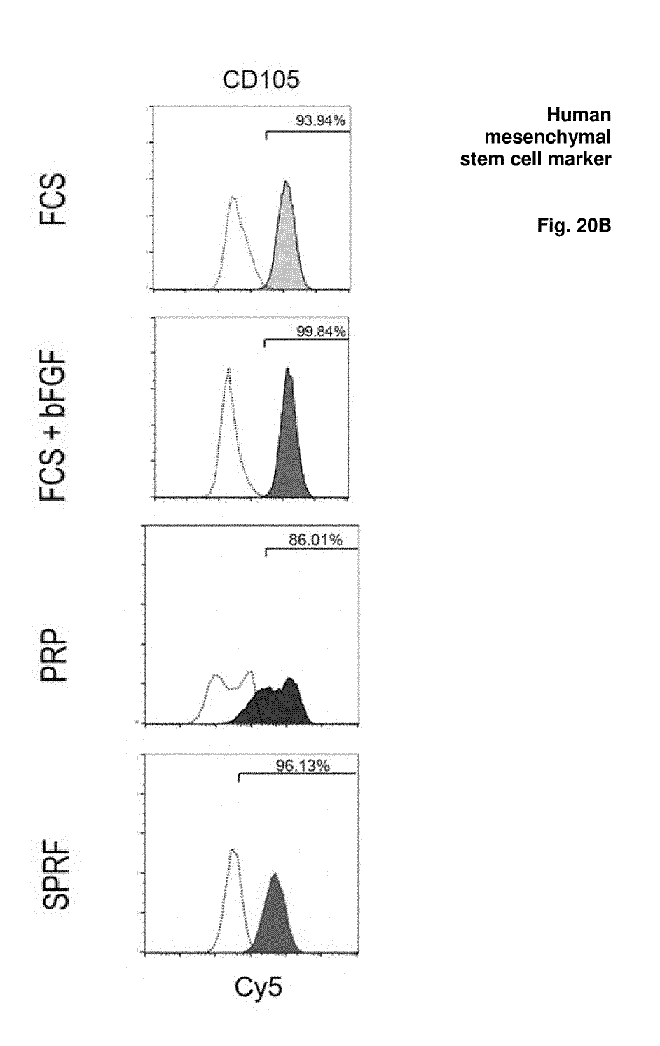

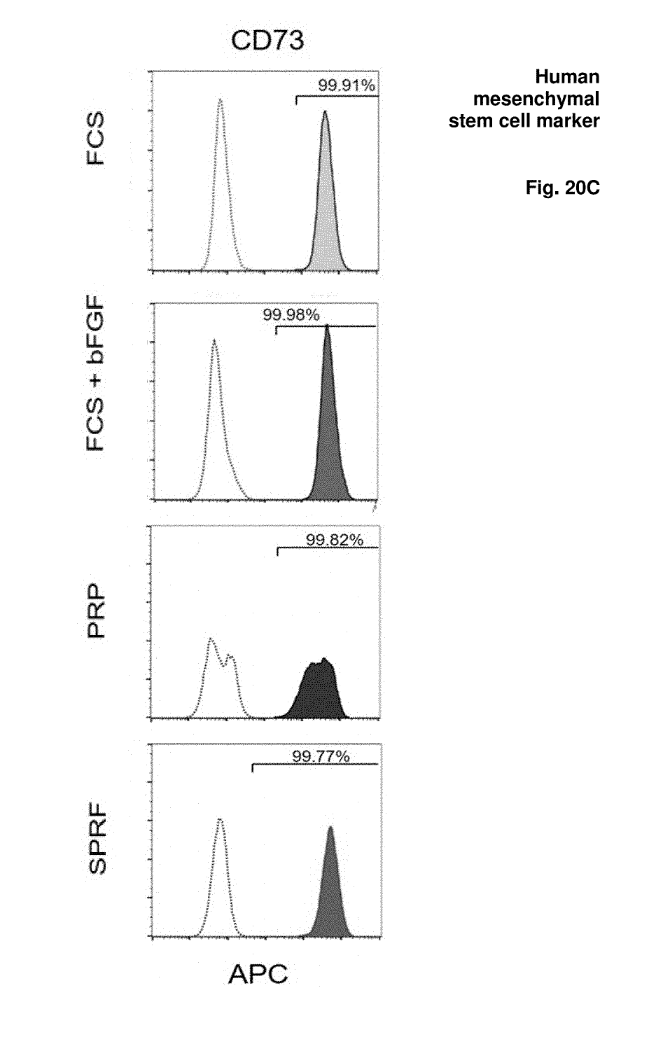

The morphology of the cells was not visibly altered by the various treatments, all preserved the typical hMSC morphology (FIG. 19).

### Example 18—Flow Cytometry Analysis of hMSC Cultured in Different Serum Supplements

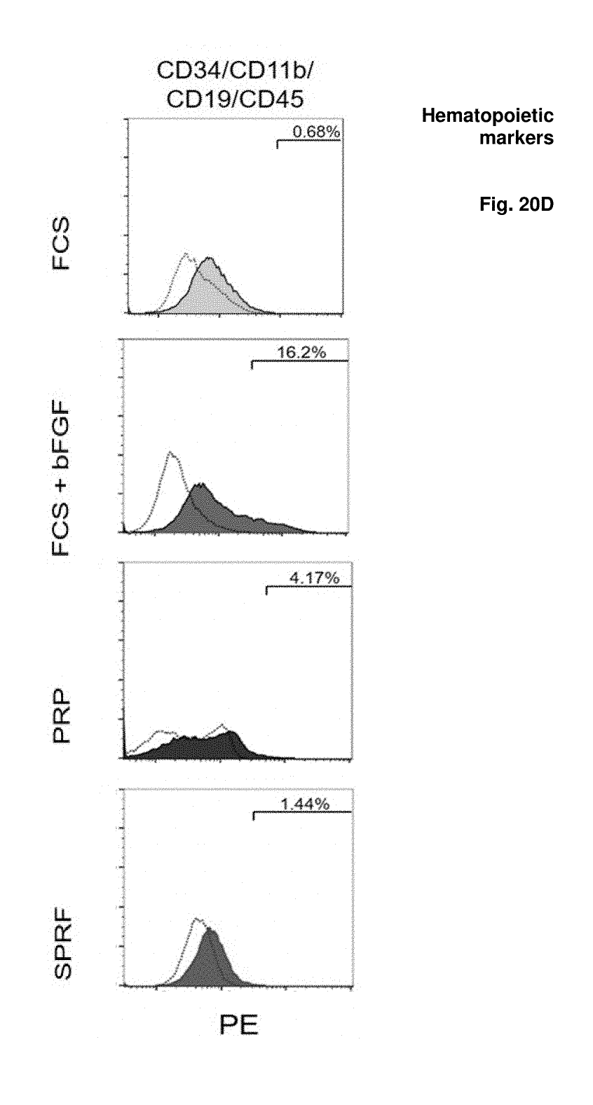

Immunophenotyping analysis was used to characterize that hMSCs cultured 5 days long in differently supplemented media preserved their hMSC-characteristic. hMSCs cultured in 10% (v/v) FCS or 10% (v/v) FCS+1 ng/mL bFGF, or 10% (v/v) PRP or 10% (v/v) SPRF were positive for CD90-FITC, CD105-PerCP-Cy5.5 and CD73-APC in more than 93.94%. While CD90-FITC and CD73-APC expression was above 99% in case of the PRP-supplemented samples (FIGS. 20A and 20C). CD105-PerCP-Cy5.5 level decreased by 13.99% and two cell populations were appeared. This thought to be the result of an unknown differentiation process in connection with PRP (FIG. 20B).

No expression of hematopoietic markers (CD34/CD11b/CD19/CD45/HLA-DR-PE) was detected in any of the experiments with 10% (v/v) FCS-, 10% (v/v) PRP- and 10% (v/v) SPRF-supplemented samples (FIG. 20D), showing that no hematopoietic differentiation occurs; however 10% (v/v) FCS+1 ng/mL bFGF supplement caused the formation of a positive subpopulation for those markers (16.2%) (FIG. 20D). This finding suggests that not hematopoietic stem cells (HSCs) but MSCs are proliferated according to the invention.

### Example 19—Gene Expression Analysis of Differently Supplemented hMSCs Cultures

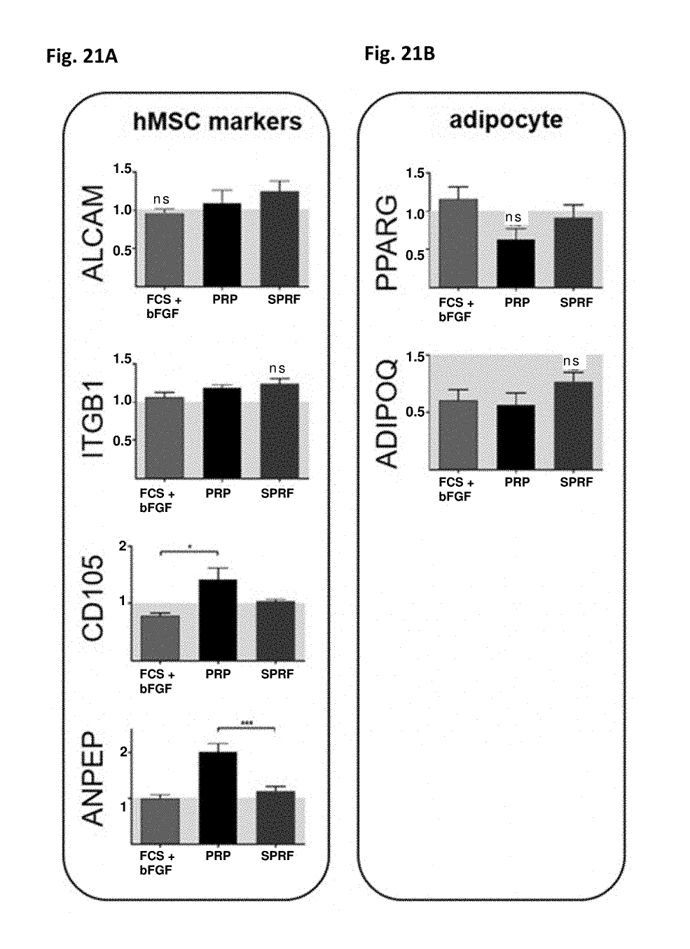

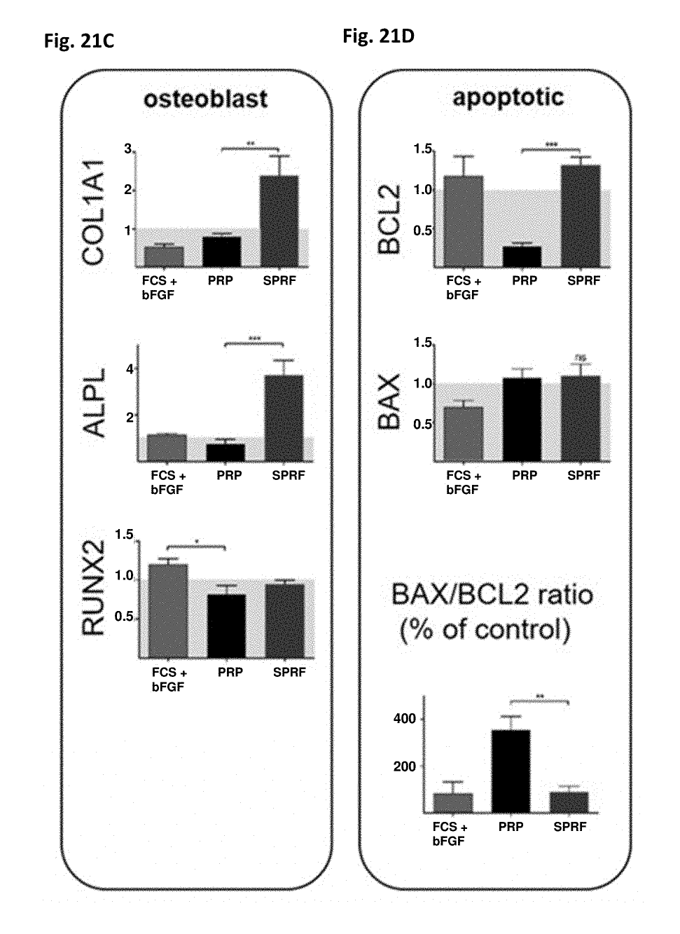

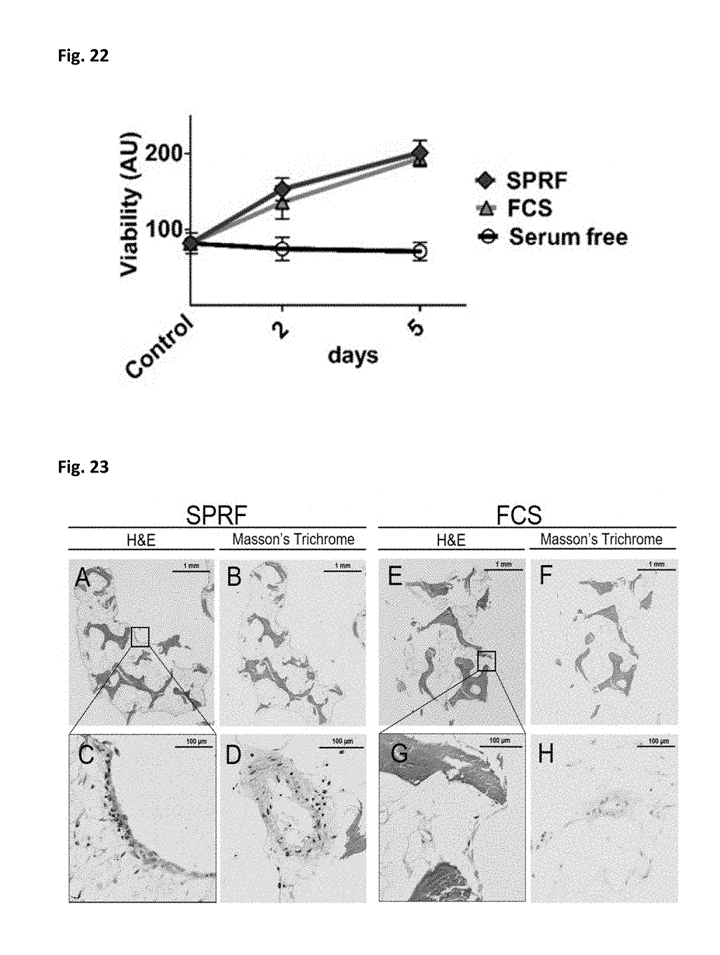

The result of this analysis is shown on FIGS. 21A-D.

FIG. 21A shows that mesenchymal stem cell marker levels remained compared to standard culture medium (10 v/v % FCS). On the Y axis the protein expression level ratio is presented, the base of comparison is the level in 10% FCS cultures.

MSCs may differentiate in either to osteoblasts or adipocytes, consequently both cell type markers have been checked to complete the study. In FIGS. 21B and 21C it is shown that PPARGG and ADIPOQ expression level did not show any change in contrast with osteoblast markers ALPL and COL1A1 which have been significantly increased in case of SPRF supplementation.

FIG. 21D. Apoptotic genes' expression levels were analyzed as well: BCL2 is antiapoptotic, while BAX is an apoptotic protein. BAX/BCL2 ratio is in equilibrium under normal physiological conditions (taken as 100% in FCS). Increase is demonstrable if cells undergo apoptotic process due to inner or under external effects. FCS+BFGF and SPRF supplementation results in an approximate 80-90%, while PRP cultured cells show a tremendous increase.

hMSC-Specific Genes Stayed Intensely Expressed in Culture of Mesenchymal Stem Cells Supplemented with 10% (v/v) SPRF

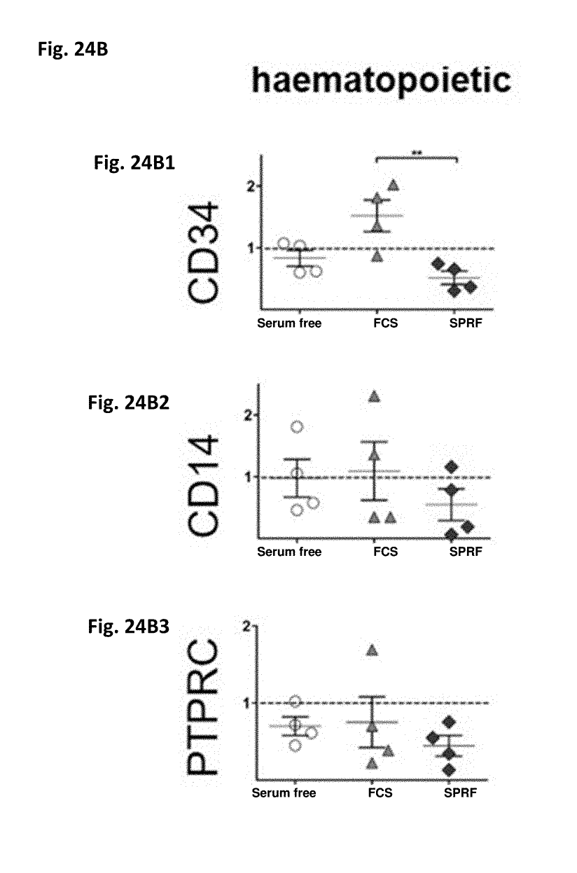

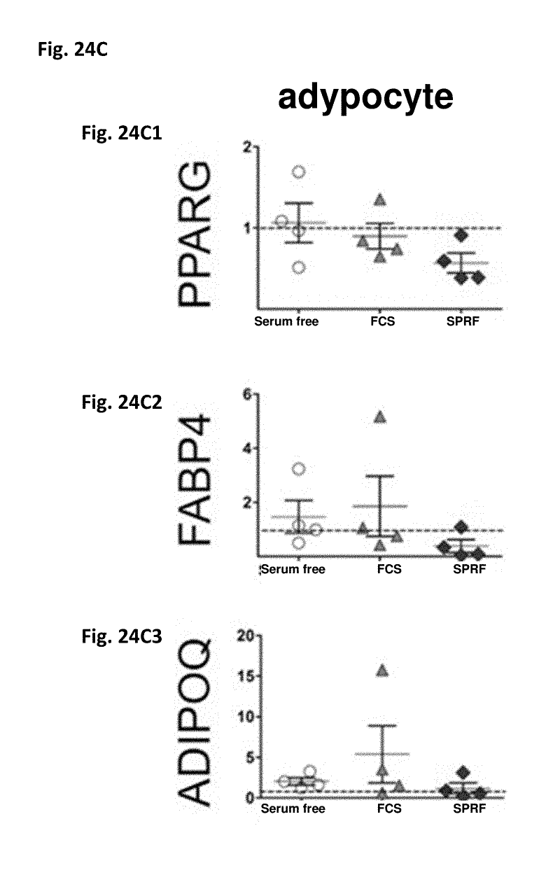

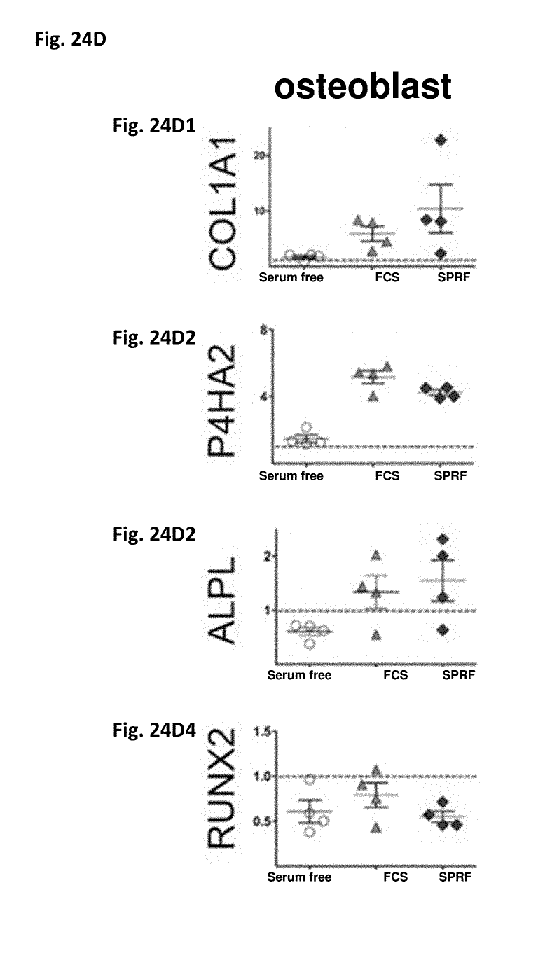

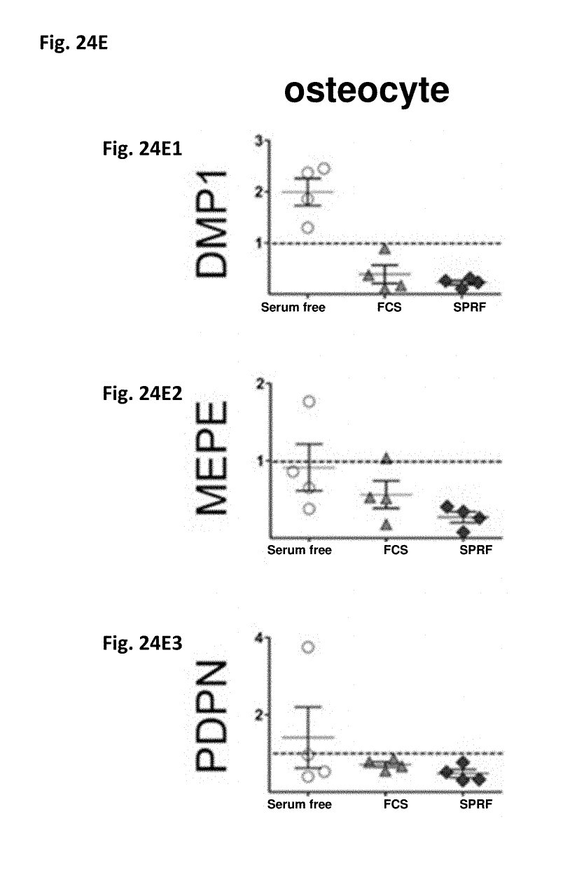

The expression of hMSC-specific genes was confirmed by real time qPCR after 5 days incubation in the media indicated above. ALCAM (CD166), ITGB1, CD105 and ANPEP expression showed significant increase in the 10% (v/v) SPRF supplemented samples when compared with 10% (v/v) FCS supplemented group 1.68-fold, 2.03-fold, 1.29-fold and 1.37-fold, respectively. While ALCAM (CD166), ITGB1, CD105 and ANPEP expressions were almost unchanged after culturing in 10% (v/v) FCS+1 ng/mL bFGF (0.95-fold, 1.35-fold, 0.98-fold and 1.29-fold, respectively), the expression of the same markers decreased in case of the 10% (v/v) PRP culturing at CD105 0.87-fold and ALCAM (CD166) 0.55-fold. Increase was found when 10% (v/v) PRP culturing in case of ITGB1 (1.67-fold) and ANPEP (2.19-fold) expression. (FIG. 24A)

Adipose Differentiation was not Observed but Osteoblastic Gene Expression was Elevated when hMSCs were Cultured in 10% (v/v) SPRF

Since hMSCs are the common progenitor cells of adipocytes and osteoblasts, adipocyte-specific and osteoblast-specific gene expression was investigated in the variously supplemented hMSC cultures by RT-qPCR. FABP4, PPARG and ADIPOQ expression, that are markers of adipogenic differentiation were not elevated when 10% (v/v) FCS supplementation was changed for 10% (v/v) FCS+1 ng/mL bFGF, 10% (v/v) PRP or 10% (v/v) SPRF, i.e. the expression level stayed unvaried compared to standard culturing method (FIG. 24C). COL1A1, ALPL and RUNX expression was slightly changed in cultures supplemented with 10% (v/v) FCS+1 ng/mL bFGF, 0.78-fold, 1.3-fold and 1.49-fold respectively. 10% (v/v) PRP had almost the same effect 1.43-fold change in COL1A1 expression, 3.11-fold change in ALPL expression and RUNX2 expression decreased 0.47-fold compared to the control group. 10% (v/v) SPRF supplement showed a significant increase in osteblastic gene expression. COL1A1 mRNA level elevated 8.38-fold, ALPL level 8.28-fold and RUNX2 level 1.31-fold compared to control (FIG. 24D).

BAX/BCL2 Ratio was Highly Increased when hMSC Culture Supplemented with 10% (v/v) PRP

BAX/BCL2 ratio was elevated 29.97-fold in case of 10% (v/v) FCS+1 ng/mL bFGF supplement, 31.99-fold when 10% (v/v) SPRF supplementation was used (FIG. 21D).

Example 20—Histological analysis of hSBPs Bone explants were fixed in 4% formalin solution. The samples were dehydrated in an ascending alcohol series at room temperature and infiltrated and embedded in a resin specifically developed for mineralized tissues (Technovit 9100 Kulzer). Infiltrated explants were placed in specific molds filled with polymerization mixture. 4 m-sections were cut using Leica RM2255 sawing microtome and stretched on slides. For hematoxylin-eosin staining the sections were immersed into hematoxylin solution and washed with 1% eosin solution. For Masson's trichrome staining sections were immersed in hematoxylin solution containing picric acid. After washing, Fuchsin Ponceau staining was performed, and unspecific parts were washed with with 1% phosphomolybdic acide solution.

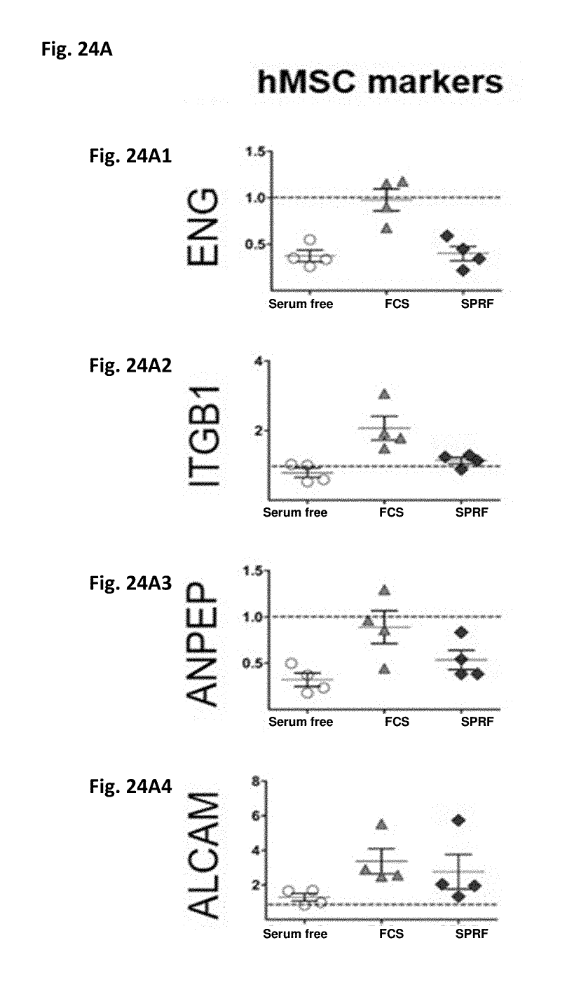

We have found that culturing hSBPs in 10% (v/v) SPRF supplemented medium for 5 days preserved bone marrow integrity as hematoxylin and eosin-stained sections (FIG. 23, A) and Masson's trichrome sections (B) show, in a surprisingly good state. However, 10% (v/v) FCS supplementation for 5 days appeared less effective and gave an inferior result in this regard. That means, SPRF revealed higher level of hMSC accumulation (C) compared to FCS (G), and preserved better the local vasculature (D,H).

Gene Expression Analysis of Human Subchondral Bone Chips

Culturing MSC on bone chips and rt-qPCR analysis were carried out as described above.

In FIG. 24A relative gene expression level on Y axis is shown compared to the values measured right after the bone chip explantation. As previously, three media were applied: serum-free medium: (◯), 10% (v/v) of FCS (Δ), 10% (v/v) SPRF (⋄).

It has been found that hMSC markers did not change in average (FIG. 24A1: ENG FIG. 24A2: ITGB1 FIG. 24A3: ANPEP FIG. 24A4: ALCAM). FIG. 24B indicates that the hematopoetic cells were not induced, however they could be present in bone chips. This indicates the selectivity of the MSC proliferation method (FIG. 24B1: CD34 FIG. 24B2: CD14 FIG. 24B3: PTPRC). Furthermore, FIG. 24C shows that adipocites were not induced in PSRF medium, thus, similarly to the MSC-cultures, adipocytes differentiation does not occur on explants either (FIG. 24C1: PPARg FIG. 24C2: FABP4 FIG. 24C3: ADPOQ).

However, surprisingly, while MSC character of the cell is maintained, an osteoblast direction differentiation can be observed on the 5^{th} day of culturing (FIG. 24D). We monitored the expression of the following osteoblast marker genes: FIG. 24D1: COL1A1 FIG. 24D2: P4HA2 FIG. 24D3: ALPL FIG. 24D4: RUNX2, among which COL1A1 and to a lesser extent ALPL seem to be upregulated.

### INDUSTRIAL APPLICABILITY

The present invention is applicable both in research and medicine among others to improve proliferation of cells or by maintaining their proliferation potential preferably for the purpose of bone regeneration or for using them in MSC cell therapy.

1. A cell culture comprising
mammalian cells,
a cell culture medium,
a serum fraction containing the fluid fraction of platelet-rich fibrin, i.e. serum fraction of PRF (SPRF),
wherein
said SPRF being obtained by a method comprising the steps of
a. separating and removing the red blood cell fraction from a venous blood sample to provide a plasma without the addition of an anticoagulant;
b. clotting said plasma to obtain a coagel of PRF spontaneously by centrifugation carried out at 1000 to 5000 g and a supernatant, wherein in said method the centrifugation is carried out for 2 to 20 minutes;
c. pressing or squeezing the coagel to obtain fluid fraction from the coagel, thereby obtaining said SPRF;
wherein said SPRF is added to the medium,
said SPRF comprising a platelet releasate from activated platelets and
said SPRF comprising a reduced content of red blood cells, platelets or fibrinogen as compared to whole blood or a reduced content of fibrin as compared to said plasma, and
wherein said SPRF is capable of inducing cell proliferation or restoring cell proliferation capacities.

2. The cell culture of claim 1 wherein said cell culture
does not comprise fetal bovine serum (FBS) or fetal calf serum (FCS), and does not comprise platelet rich plasma (PRP) and does not comprise any other growth factor either, only those which are present in the SPRF.

3. The cell culture according to claim 1 wherein said SPRF is depleted in a growth factor selected from the group consisting of PDGF-AB, PDGF-BB and TGF beta-1
as compared to platelet rich plasma (PRP).

4. The cell culture according to claim 1 wherein said cells are mammalian cells.

5. The cell culture according to claim 4 wherein said mammalian cells are selected from the group consisting of stem cells, epithelial cells, cells of the periosteum, osteogenic cells, angiogenic cells, stromal cells, mesenchymal cells, e.g. mesenchymal cells of bone marrow, adipose tissue, microvascular tissue or other mesenchymal tissue origin, osteoprogenitor cells and bone cells.

6. The cell culture of claim 1 wherein in said cell culture the SPRF is prepared by a method
wherein centrifugation is carried out at 1000 to 2000 g.

7. The cell culture of claim 1 wherein
said medium comprises 2-20% (v/v), preferably 5-15% (v/v), highly preferably 8 to 12% (v/v) or about 10% (v/v) SPRF and
wherein said medium comprises besides SPRF, no FBS (FCS) and no other serum derived product or supplement and preferably no other growth factors.

8. The cell culture of claim 7 wherein
the medium is a derivative of Dulbecco's modified Eagle's medium (DMEM) which differs from DMEM in that it is supplemented with 2-20% (v/v), preferably 5-15% (v/v), highly preferably with 8 to 12% (v/v) or about 10% (v/v) SPRF and comprises no other serum derived product or supplement and no other growth factors.

9. The cell culture of claim 7 wherein
the cells in the culture are MSCs that are contacted or maintained in contact with SPRF, preferably for until at least a time-period when osteoblast direction differentiation occurs, preferably for until at least a time-period when the expression of at least one, preferably two or at least two osteoblast specific marker gene(s) is/are increased in a medium supplemented with SPRF.

10. The cell culture of claim 7 wherein
the cells are chondrocytes and
SPRF is added to the culturing medium of chondrocytes.

11. A method for using an isolated serum fraction of platelet rich fibrin (SPRF) as a cell culture additive, said method comprising the steps of
culturing cells by incubating said cells in a medium, and
adding the SPRF to the medium,
said SPRF being obtained by a method comprising the steps of
a. separating and removing the red blood cell fraction from a venous blood sample without the addition of an anticoagulant, to provide a plasma;
b. clotting said plasma to obtain a coagel of PRF spontaneously by centrifugation carried out at 1000 to 5000 g and a supernatant, wherein in said method the centrifugation is carried out for 2 to 20 minutes;
c. pressing or squeezing the coagel to obtain fluid fraction from the coagel, thereby obtaining said SPRF;
said SPRF comprising a platelet releasate from activated platelets and
said SPRF comprising a reduced content of red blood cells, platelets or fibrinogen as compared to whole blood or a reduced content of fibrin as compared to said plasma, and
wherein said SPRF is capable of inducing cell proliferation or restoring cell proliferation capacities.

12. The method of claim 11 for using an isolated serum fraction of platelet rich fibrin (SPRF) as
a cell culture additive, said method comprising the steps of
contacting the isolated serum fraction with a culture of cells in vitro,
incubating said cells in vitro for a period of time sufficient to promote cell growth or regeneration,
thereby promoting proliferation of cells.

13. The method of claim 11 wherein said medium or said cell culture
does not comprise fetal bovine serum (FBS) or fetal calf serum (FCS), and does not comprise platelet rich plasma (PRP) and does not comprise any other growth factor either, only those which are present in the SPRF,
wherein said SPRF enhances the proliferation rate of said cells in vitro, ex vivo or in vivo, while maintaining their potential to differentiate into several cell types.

14. The method according to claim 11 wherein said isolated serum fraction is depleted in a growth factor selected from the group consisting of PDGF-AB, PDGF-BB and TGF beta-1
as compared to said plasma or whole blood.

15. The method according to claim 11 wherein said cells are mammalian cells.

16. The method according to claim 15 wherein said mammalian cells are selected from the group consisting of stem cells, epithelial cells, cells of the periosteum, osteogenic cells, angiogenic cells, stromal cells, mesenchymal cells of bone marrow, adipose tissue, microvascular tissue or other mesenchymal tissue origin, osteoprogenitor cells, bone cells and chondrocytes.

17. The method according to claim 11 wherein the serum fraction is freshly prepared and ready-for-use.

18. The method according to claim 11 wherein the serum fraction is provided in an application device.

19. The method according to claim 11 wherein the serum fraction is prepared as an autologous pharmaceutical or medicinal product.

20. The method according to claim 11, wherein the cells in the medium comprising SPRF as a cell culture additive are administered to a patient.

21. The method according to claim 11 for increasing proliferation rate of dedifferentiated chondrocytes comprising contacting a serum fraction of platelet rich fibrin (SPRF) with dedifferentiated chondrocytes, said SPRF being prepared from whole blood obtained from one or more donor subject(s).

22. The method according to claim 11 wherein SPRF does not redifferentiate chondrocytes from the dedifferentiated state or provides a lesser extent of redifferentiation than PRP, preferably measured by the col II/col I ratio.

23. The method according to claim 11 wherein SPRF obtained from a donor subject is used for increasing proliferation rate of chondrocytes in vitro before transplantation thereof, wherein preferably SPRF is applied in the cell culture in a concentration between 1-25% or 2-20%, preferably 5 to 15%, highly preferably 8 to 12% or in particular about 10%, wherein the percentage of concentration is given in v/v %.

24. The method according to claim 11 wherein said method is a method of transplantation or implantation of chondrocytes into a patient in need thereof, wherein said SPRF is an SPRF prepared from whole blood obtained from a donor subject and wherein said SPRF is contacted with the dedifferentiated chondrocytes to be transplanted or implanted to said patient in vitro, to use for increasing proliferation rate of said chondrocytes.

25. The method according to claim 24 wherein the patient is a subject in need of cartilage repair, in particular articular cartilage repair and/or cartilage replacement therapy, preferably articular cartilage repair,
or in more particular the patient is a subject with cartilage failure, osteoarthritis, cartilage damage, osteochondral damage, rheumatoid arthritic damage, autoimmune arthritis, reactive arthritis,
cellular matrix linkage rupture, chondrocyte protein synthesis inhibition, and chondrocyte apoptosis, a condition requiring cartilage regeneration in particular in cartilage ulcer, osteoarthritis or traumatic cartilage loss, a condition requiring subchondral bone regeneration in osteoarthritis, Ahlback's disease or osteochondral lesions.

26. The method according to claim 11 for use of serum fraction of platelet rich fibrin (SPRF) for selectively increasing MSC proliferation rate in vitro, in vivo or ex vivo wherein said differentiated MSCs maintain their potential to differentiate into several cell types, preferably MSCs are obtained from a subject.

27. The method according to claim 26, said method comprising
i. providing SPRF,
ii. adding SPRF to a pool of MSCs,
iii. allowing MSCs to proliferate,
for at least 5 days.

28. The method according to claim 26 for use of SPRF as a cell medium supplement instead of PRP and FBS wherein said SPRF enhances the proliferation rate of human mesenchymal stem cells in vitro, ex vivo or in vivo, while maintaining their potential to differentiate into several cell types, wherein upon proliferation of MSCs expression of one or both of the following osteogenic marker genes is increased: COL1A1 and ALPL, and wherein
said medium comprises SPRF, preferably 2-20% (v/v), preferably 5-15% (v/v), highly preferably 8 to 12% (v/v) or about 10% (v/v) SPRF, as a supplement and
the medium does not comprise fetal bovine serum (FBS) or fetal calf serum (FCS), and does not comprise platelet rich plasma (PRP) and preferably does not comprise FGF (e.g. bFGF) and preferably does not comprise any other growth factor either, only those which are present in the SPRF.

29. The method according to claim 26 for use of SPRF in therapy, preferably in stem cell therapy, wherein in said therapy SPRF obtained from a donor subject is used to increase proliferation rate of the patient's MSCs expanded in vitro, ex vivo or in vivo, wherein the MSCs so proliferated maintain their undifferentiated character with the potential to differentiate into several cell types,
wherein proliferation of MSCs is carried out for at least 5 days.

30. The method according to claim 26 wherein the MSCs are bone marrow derived mesenchymal stem cells (BM-MSCs or bone marrow stromal stem cells), or
the MSCs are adipose derived mesenchymal stem cells (AD-MSCs), wherein
preferably the MSC culturing medium normally comprises a carbon source, preferably a sugar source and preferably a glutamine source and preferably pyruvate.

31. A medium comprising
a cell culture medium and
a serum fraction containing the fluid fraction of platelet-rich fibrin, i.e. serum fraction of PRF (SPRF),
wherein
said SPRF being obtained by a method comprising the steps of
a. separating and removing the red blood cell fraction from a venous blood sample to provide a plasma without the addition of an anticoagulant;
b. clotting said plasma to obtain a coagel of PRF spontaneously by centrifugation carried out at 1000 to 5000 g and a supernatant, wherein in said method the centrifugation is carried out for 2 to 20 minutes;
c. pressing or squeezing the coagel to obtain fluid fraction from the coagel, thereby obtaining said SPRF;
wherein said SPRF is added to the cell culture medium,
said SPRF comprising a platelet releasate from activated platelets and
said SPRF comprising a reduced content of red blood cells, platelets or fibrinogen as compared to whole blood or a reduced content of fibrin as compared to said plasma, and
wherein said SPRF is capable of inducing cell proliferation or restoring cell proliferation capacities.

32. The medium, according to claim 31 wherein said cell culture medium
does not comprise fetal bovine serum (FBS) or fetal calf serum (FCS), and does not comprise platelet rich plasma (PRP) and does not comprise any other growth factor either, only those which are present in the SPRF.

33. The medium according to claim 31 wherein said isolated SPRF is depleted in a growth factor selected from the group consisting of PDGF-AB, PDGF-BB and TGF beta-1
as compared to platelet rich plasma (PRP).

34. The medium according to claim 31 wherein said cell culture medium is a stem cell culture medium.

35. The medium according to claim 31 wherein said mammalian cells are selected from the group consisting of stem cells, epithelial cells, cells of the periosteum, osteogenic cells, angiogenic cells, stromal cells, mesenchymal cells of bone marrow, adipose tissue, microvascular tissue or other mesenchymal tissue origin, osteoprogenitor cells, bone cells and chondrocytes.

36. The medium according to claim 31 wherein in said cell culture the SPRF is prepared by a method wherein centrifugation is carried out at 1000 to 2000 g.

37. The medium of claim 31 wherein
said medium comprises 2-20% (v/v), preferably 5-15% (v/v), highly preferably 8 to 12% (v/v) or about 10% (v/v) SPRF and
wherein said medium comprises besides SPRF no FBS (FCS) and no other serum derived product or supplement and preferably no other growth factors.

38. The medium of claim 37 wherein
the cell culture medium is a derivative of Dulbecco's modified Eagle's medium (DMEM) which differs from DMEM in that it is supplemented with 2-20% (v/v), preferably 5-15% (v/v), highly preferably with 8 to 12% (v/v) or about 10% (v/v) SPRF and
said medium comprises no other serum derived product or supplement and no other growth factors.
